SEQUENCE LISTING (156 sequences)

SEQ ID NO: 1 - DNA, 191 bp, Homo sapiens
  source 1..191: mol_type = genomic DNA
gttgttgtttgagggtatggaygatgtgygggatggaggtttagagttgtttatttttgtaattaatgtttaygtaattattagggggygaaaggattttaattttataygtagtgagtggtttttaygttaygttttagtaggagaaatgaagttttyggggayggaagaagatggtagttatttggtgt

SEQ ID NO: 2 - DNA, 191 bp, Homo sapiens
  source 1..191: mol_type = genomic DNA
gggtaaattgaggttttagtttagygayggttaygyggagggggggygagtgggtttagagggggttaygggttaggggaaygygagttaggttagatttagayggygattttgggayggtggttatggtaggtygagaygttgygygygaaygtatattyggagayggagtagttataaaattaggtttg

SEQ ID NO: 3 - DNA, 175 bp, Homo sapiens
  source 1..175: mol_type = genomic DNA
ttagaggaygttggagtaggaggaaygggggagtgygatgtggggygtgygttttttggagaagaaaggygggaygttyggggttgttttttygtttttyggagtttttagggaaygttgtttggatatagtagygygggygttttataatttgagggttygtaggattttggga

SEQ ID NO: 4 - DNA, 180 bp, Homo sapiens
  source 1..180: mol_type = genomic DNA
gtgygataggagttaggtgggttyggygyggagattygygggagtygggtygyggygggagygyggtaggyggagaggttygyggaggtagttaggttyggygagaaaggttaaaattttttggttttattygtagtgttttattygggtayggtttgtgggattagtgtattyggggag

SEQ ID NO: 5 - DNA, 151 bp, Homo sapiens
  source 1..151: mol_type = genomic DNA
gggttttggataaggttgggttttyggtttyggttttattatttttatttyggattygtttgggggttttttygttagygttttattttygttttaaagatttaayggtgttaaagtygttttagtgaagagtagtatgttttgatttgga

SEQ ID NO: 6 - DNA, 108 bp, Homo sapiens
  source 1..108: mol_type = genomic DNA
gggagtaggtgtaggtattgggygtttggggaaggygagtaggtgygagagtaggyggtaggtttgagaggygttggygygygttgggataaaaatagagtgggaagg

SEQ ID NO: 7 - DNA, 212 bp, Homo sapiens
  source 1..212: mol_type = genomic DNA
ggttgaatttgtattttgtatagaattttaaaaagtttttttgattgttgtttatttatttaaaaaagtaaagtattgtyggtatttttttgaaaataaataatttaggtattyggtgtttttatatgtaatttattaatagtaatggataattttttaaagttataaatagtattgggagttygattttaagaagttattaattttaagat

SEQ ID NO: 8 - DNA, 156 bp, Homo sapiens
  source 1..156: mol_type = genomic DNA
gggaatygatttttagttgtgttaatttgttttagtttttttaagatttttttttttaattaaagtagggagagtttttttatgatttggtgatgttattaaygygggygtgttygygaggtagagttyggttgtygyggaatttggaggtttggg

SEQ ID NO: 9 - DNA, 275 bp, Homo sapiens
  source 1..275: mol_type = genomic DNA
ggagatttggaagaggtaggtagtygagtttatatygttggagaygttgtattygtagttgtygttgttgtygygagttayggygtygaggygtagtttygygtgattyggygtttyggyggyggygggaataataggyggyggyggtagtagttgttttttgaaaygttatatagtygaggygatgygttgggggttgtttygtagtagygagtagygtaggagtaygggtyggtttagygtttggygtaygttttgggaattgggttttattt

SEQ ID NO: 10 - DNA, 178 bp, Homo sapiens
  source 1..178: mol_type = genomic DNA
gattttggagggtttttagagttggggagtagttygttygttttgtgttttaatttttttagtttgggttttagtatttygattggggtygygtgygygtygggggattayggtygttaggtattgttatttgyggaggyggagaagygaagyggyggtaagaggaaaagcgatagtt

SEQ ID NO: 11 - DNA, 211 bp, Homo sapiens
  source 1..211: mol_type = genomic DNA
gagacgggttttattatgttgattaggttggttttaaatttttgattttaggtgatttgttygtttyggttttttaaagtgttggtattataggygygagtygtagtgtagggtttttygyggatttatttttttttattattattagggyggygtyggagatttttaggattttattttgtttaaaatttgattttttatagttgggggt

SEQ ID NO: 12 - DNA, 200 bp, Homo sapiens
  source 1..200: mol_type = genomic DNA
ttygggagaattgtttggggtagagggggtaggagaagygtttttygttygtgtgygttttgtagtggygggttagttygtyggaaygygtaaattttttgtygtagtygagttagtygtaggagaaagggygtttattygtgtgggtgygttggtgggatttgaggtgygaygatttgtaataggttttggtgtagttt

SEQ ID NO: 13 - DNA, 170 bp, Homo sapiens
  source 1..170: mol_type = genomic DNA
ggtgaaattggatttttaagtttgtgtaggtgaaggtgtgtgagagygttygagatggtagaataagagttataggtaattttgttttttygtttttttttattattttygttygttygttygttygygtttttatttagtyggaaaggtgggataagggggggtttttt

SEQ ID NO: 14 - DNA, 127 bp, Homo sapiens
  source 1..127: mol_type = genomic DNA
tgttaggttgggtttttaagtygggtygttygygtagagttygggyggagttggggggtgtggggggaatgttyggggtaggattygttttygygattagttttgggagygaagtgggaaggggtag

SEQ ID NO: 15 - DNA, 203 bp, Homo sapiens
  source 1..203: mol_type = genomic DNA
gaagtagtatttggatttgagttyggggaygggtaaaggaaygtagttygtgagtggtttagagagygggaattagagygtttygayggtagyggaagttatygygggygttaaattagtaaygygttttttgaggataggaggttayggygtaaaagtagattgggttyggaaataygtgttttataaatggggaaatgagt

SEQ ID NO: 16 - DNA, 143 bp, Homo sapiens
  source 1..143: mol_type = genomic DNA
gggggatgggaagtatyggygggtggaggttggaatygaaataggaaagggagttggaagyggygtttagagttgggygagtaggggaaggggatttagygtttgygyggttttyggyggggyggattgtaggtaggygtttt

SEQ ID NO: 17 - DNA, 131 bp, Homo sapiens
  source 1..131: mol_type = genomic DNA
gagagtttggtggttttggttagtatttyggatagggattygggygttaatyggtagatgygttgygttttttattggtaggtgtattttyggttgtagygggtttaygygggtagttgtttggtggtgat

SEQ ID NO: 18 - DNA, 162 bp, Homo sapiens
  source 1..162: mol_type = genomic DNA
ggattagggtatgtaaaaaagatatygatataatggaaaagaaattttygaaggtagaatttygtygttygygtygygtygygtygtttagggtygggtttygygygtttygygtygtygtygtagtttttygyggtagtagtaggagtagtagtgttyggt

SEQ ID NO: 19 - DNA, 167 bp, Homo sapiens
  source 1..167: mol_type = genomic DNA
tgagtttagggttttttattttatyggtttygttttyggtttyggttttagtttyggttttagtttyggtttttgygggatygtyggygaataygtttyggtgtatggyggyggygtagtygaagttaaaggggtygtttaggygtattagtagttggtgtaggaag

SEQ ID NO: 20 - DNA, 165 bp, Homo sapiens
  source 1..165: mol_type = genomic DNA
ggagtygttagaaggtggggayggtttyggaagtgggggttygggtyggattttygggygttttyggygtygttttttygtttagttttyggyggtttttgtygatggttaggyggggtygatygyggtttaggtygtttaggagggagtaggtttggtagagyg

SEQ ID NO: 21 - DNA, 21 bp, Homo sapiens
  source 1..21: mol_type = genomic DNA
gttgttgtttgagggtatgga

SEQ ID NO: 22 - DNA, 20 bp, Homo sapiens
  source 1..20: mol_type = genomic DNA
gggtaaattgaggttttagt

SEQ ID NO: 23 - DNA, 25 bp, Homo sapiens
  source 1..25: mol_type = genomic DNA
ttagaggaygttggagtaggaggaa

SEQ ID NO: 24 - DNA, 23 bp, Homo sapiens
  source 1..23: mol_type = genomic DNA
gtgygataggagttaggtgggtt

SEQ ID NO: 25 - DNA, 24 bp, Homo sapiens
  source 1..24: mol_type = genomic DNA
gggttttggataaggttgggtttt

SEQ ID NO: 26 - DNA, 22 bp, Homo sapiens
  source 1..22: mol_type = genomic DNA
gggagtaggtgtaggtattggg

SEQ ID NO: 27 - DNA, 24 bp, Homo sapiens
  source 1..24: mol_type = genomic DNA
ggttgaatttgtattttgtataga

SEQ ID NO: 28 - DNA, 23 bp, Homo sapiens
  source 1..23: mol_type = genomic DNA
gggaatagatttttagttgtgtt

SEQ ID NO: 29 - DNA, 24 bp, Homo sapiens
  source 1..24: mol_type = genomic DNA
ggagatttggaagaggtaggtagt

SEQ ID NO: 30 - DNA, 20 bp, Homo sapiens
  source 1..20: mol_type = genomic DNA
gattttggagggtttttaga

SEQ ID NO: 31 - DNA, 27 bp, Homo sapiens
  source 1..27: mol_type = genomic DNA
ygggttttattatgttgattaggttgg

SEQ ID NO: 32 - DNA, 25 bp, Homo sapiens
  source 1..25: mol_type = genomic DNA
ttygggagaattgtttggggtagag

SEQ ID NO: 33 - DNA, 21 bp, Homo sapiens
  source 1..21: mol_type = genomic DNA
ggtgaaattggatttttaagt

SEQ ID NO: 34 - DNA, 20 bp, Homo sapiens
  source 1..20: mol_type = genomic DNA
tgttaggttgggtttttaag

SEQ ID NO: 35 - DNA, 23 bp, Homo sapiens
  source 1..23: mol_type = genomic DNA
gaagtagtatttggatttgagtt

SEQ ID NO: 36 - DNA, 16 bp, Homo sapiens
  source 1..16: mol_type = genomic DNA
gggggatgggaagtat

SEQ ID NO: 37 - DNA, 25 bp, Homo sapiens
  source 1..25: mol_type = genomic DNA
gagagtttggtggttttggttagta

SEQ ID NO: 38 - DNA, 26 bp, Homo sapiens
  source 1..26: mol_type = genomic DNA
tgagtggygttagtgtaggtttaggt

SEQ ID NO: 39 - DNA, 23 bp, Homo sapiens
  source 1..23: mol_type = genomic DNA
tgagtttagggttttttatttta

SEQ ID NO: 40 - DNA, 21 bp, Homo sapiens
  source 1..21: mol_type = genomic DNA
ggagtygttagaaggtgggga

SEQ ID NO: 41 - DNA, 25 bp, Homo sapiens
  source 1..25: mol_type = genomic DNA
acacaccaaataactaccatcttct

SEQ ID NO: 42 - DNA, 24 bp, Homo sapiens
  source 1..24: mol_type = genomic DNA
caaacctaattttataactactcc

SEQ ID NO: 43 - DNA, 25 bp, Homo sapiens
  source 1..25: mol_type = genomic DNA
tcccaaaatcctacraaccctcaaa

SEQ ID NO: 44 - DNA, 25 bp, Homo sapiens
  source 1..25: mol_type = genomic DNA
ctccccraatacactaatcccacaa

SEQ ID NO: 45 - DNA, 27 bp, Homo sapiens
  source 1..27: mol_type = genomic DNA
tccaaatcaaaacatactactcttcac

SEQ ID NO: 46 - DNA, 20 bp, Homo sapiens
  source 1..20: mol_type = genomic DNA
tccactctccttcccactct

SEQ ID NO: 47 - DNA, 25 bp, Homo sapiens
  source 1..25: mol_type = genomic DNA
ccaaaaaactcaatactcatatatc

SEQ ID NO: 48 - DNA, 17 bp, Homo sapiens
  source 1..17: mol_type = genomic DNA
cccaaacctccaaattc

SEQ ID NO: 49 - DNA, 23 bp, Homo sapiens
  source 1..23: mol_type = genomic DNA
aaataaaacccaattcccaaaac

SEQ ID NO: 50 - DNA, 21 bp, Homo sapiens
  source 1..21: mol_type = genomic DNA
aactatcccttttcctcttac

SEQ ID NO: 51 - DNA, 26 bp, Homo sapiens
  source 1..26: mol_type = genomic DNA
acaaaacccccaactataaaaaatca

SEQ ID NO: 52 - DNA, 24 bp, Homo sapiens
  source 1..24: mol_type = genomic DNA
raactacaccaaaacctattacaa

SEQ ID NO: 53 - DNA, 18 bp, Homo sapiens
  source 1..18: mol_type = genomic DNA
aaaaaacccccccttatc

SEQ ID NO: 54 - DNA, 16 bp, Homo sapiens
  source 1..16: mol_type = genomic DNA
ctaccccttcccactt

SEQ ID NO: 55 - DNA, 19 bp, Homo sapiens
  source 1..19: mol_type = genomic DNA
actcatttccccatttata

SEQ ID NO: 56 - DNA, 19 bp, Homo sapiens
  source 1..19: mol_type = genomic DNA
aaaacccctacctacaatc

SEQ ID NO: 57 - DNA, 21 bp, Homo sapiens
  source 1..21: mol_type = genomic DNA
atcaccaccaaacaactaccc

SEQ ID NO: 58 - DNA, 24 bp, Homo sapiens
  source 1..24: mol_type = genomic DNA
raacactactactcctactactac

SEQ ID NO: 59 - DNA, 23 bp, Homo sapiens
  source 1..23: mol_type = genomic DNA
cttcctacaccaactactaatac

SEQ ID NO: 60 - DNA, 25 bp, Homo sapiens
  source 1..25: mol_type = genomic DNA
crctctaccaaacctactccctcct

SEQ ID NO: 61 - DNA, 57 bp, Homo sapiens
  source 1..57: mol_type = genomic DNA
cgcggccctgggtcccatttctggcatgtccatctgtcatcacagctcctacctccg

SEQ ID NO: 62 - DNA, 180 bp, Homo sapiens
  source 1..180: mol_type = genomic DNA
cggggaagtctgagactgcagtgcgtggtgatcacaacactgcactccagcctgagcaacagagtgagaccatgtctaaaaataaataaataaataaaaatgccgggcgtggtggctcaagcctgtaatcccagcactttgggaggccaaggtgggcagatcacttgaggtcaggagttc

SEQ ID NO: 63 - DNA, 77 bp, Homo sapiens
  source 1..77: mol_type = genomic DNA
cgggccagatgcgcctgagcgcggcctcgttatgtattcatgagctgtgaggaaaagaaataaaaggattcattatc

SEQ ID NO: 64 - DNA, 119 bp, Homo sapiens
  source 1..119: mol_type = genomic DNA
cggattaaaaaaaaaattccccactttctttccctctcggcaatttatcggacttcccccctccagctcttaaattagtgagatgtggtcacataaagtaccttaaacaggctgtcccg

SEQ ID NO: 65 - DNA, 65 bp, Homo sapiens
  source 1..65: mol_type = genomic DNA
cgcgatctcggcccactgcaacctccgcctcccaggttcaggcgattctccagcctcagcctccg

SEQ ID NO: 66 - DNA, 62 bp, Homo sapiens
  source 1..62: mol_type = genomic DNA
cgtgatccacccacctcggcctctcaaagtgctgggattacagccatgagccaccacacccg

SEQ ID NO: 67 - DNA, 62 bp, Homo sapiens
  source 1..62: mol_type = genomic DNA
cgtgatctgcctgccttggcctcccaaagtgctgggattacaggtgtgagccactgggcccg

SEQ ID NO: 68 - DNA, 29 bp, Homo sapiens
  source 1..29: mol_type = genomic DNA
cgccgctccccgagaggccgcaaaagccc

SEQ ID NO: 69 - DNA, 150 bp, Homo sapiens
  source 1..150: mol_type = genomic DNA
cggatctccagtttttcggtgtaccaagcagacctattttacctccatggggagcaatttcagttctgggttagctagggtcaggaagcatgggaagggaaggggaacaaagtgagcaggagctgagtcctgaggcctcttgtccccttc

SEQ ID NO: 70 - skipped

SEQ ID NO: 71 - DNA, 71 bp, Homo sapiens
  source 1..71: mol_type = genomic DNA
cgtgaccccctctgggcccactcgccccccctccgcgtggccgtcgctgaactggggcctcagtttacccg

SEQ ID NO: 72 - DNA, 312 bp, Homo sapiens
  source 1..312: mol_type = genomic DNA
cgctgccgctgccgacctttttggcccttacctcacgtcccagggtcctgcgggccctcaagttgtggggcgcccgcgctgctgtgtccagacagcgttccctgagagctccgggaagcgggaagacagccccgggcgtcccgcctttcttctccagaaaacgcacgccccacatcgcactcccccgttcctcctgctccagcgtcctctggtccttctttctgtctgtgcctccgtctttgtctcaacctctcaggcttgctcgctccctgcccagattttgtggcccaggctcctggctgtctgactccg

SEQ ID NO: 73 - DNA, 76 bp, Homo sapiens
  source 1..76: mol_type = genomic DNA
cgcttctctggggaccgcctcttggggccgttggcggctgccgcgcgctcggcctgcgcgtccctccgtccctccg

SEQ ID NO: 74 - DNA, 55 bp, Homo sapiens
  source 1..55: mol_type = genomic DNA
cgacatgcagacagtagtcggctgacattttttgctatttccgttattacagccg

SEQ ID NO: 75 - DNA, 31 bp, Homo sapiens
  source 1..31: mol_type = genomic DNA
cgcagctgacaaacagggcggcttgtcgccg

SEQ ID NO: 76 - DNA, 57 bp, Homo sapiens
  source 1..57: mol_type = genomic DNA
cgccagcgcctctcaggcctgccgcctgctctcgcacctgctcgccttccccaggcg

SEQ ID NO: 77 - DNA, 79 bp, Homo sapiens
  source 1..79: mol_type = genomic DNA
cgcgccctcggcttccctgttgctcagggttatttcttcctgccatcagctggagaagcgctctccgaatatttccccg

SEQ ID NO: 78 - DNA, 51 bp, Homo sapiens
  source 1..51: mol_type = genomic DNA
cggggccacctgtgccctcttcccagagcactcgaggccaggcacgatccg

SEQ ID NO: 79 - DNA, 22 bp, Homo sapiens
  source 1..22: mol_type = genomic DNA
cgagaataagggctcggctccg

SEQ ID NO: 80 - DNA, 74 bp, Homo sapiens
  source 1..74: mol_type = genomic DNA
cggagaaggccgaggacgacgaggagatcgacctggaaagcatcgacattgacaagatcgacgagcacgatggc

SEQ ID NO: 81 - DNA, 64 bp, Homo sapiens
  source 1..64: mol_type = genomic DNA
cggaggctgaggcagaagaatcgattgaactcaggaggcagaggttgcagtgagccaagatcgc

SEQ ID NO: 82 - DNA, 56 bp, Homo sapiens
  source 1..56: mol_type = genomic DNA
cgagctctcctgggccgacctagatttccactgccacatactttccgctccctgcg

SEQ ID NO: 83 - DNA, 202 bp, Homo sapiens
  source 1..202: mol_type = genomic DNA
cgctgtaaacaggggcgcgggccggagagcgggtgtgcaaagtgggcgcagggccctggggccgcgccccttgctctgccggctcgactcttgcacggcgggcggtgaggagggggctgttcgcccagacagagggccacctcctagcccgggagcagagcagagggcctgggcctgcagctaagctcaaggctggggtgtt

SEQ ID NO: 84 - DNA, 78 bp, Homo sapiens
  source 1..78: mol_type = genomic DNA
cgggaggagctctgtgctcagagcccgcggtgtctggtgaactttaaagtcctggttgaagacagagtgaaactgtac

SEQ ID NO: 85 - DNA, 79 bp, Homo sapiens
  source 1..79: mol_type = genomic DNA
cggttctccacttattacacatattattactttgctcagtgtgtctccccatacccaatgccttcgaattgatgacccg

SEQ ID NO: 86 - DNA, 36 bp, Homo sapiens
  source 1..36: mol_type = genomic DNA
cggtatttccctgaaaataaataatccaggcatccg

SEQ ID NO: 87 - DNA, 28 bp, Homo sapiens
  source 1..28: mol_type = genomic DNA
cgcgggcgtgctcgcgaggcagagcccg

SEQ ID NO: 88 - DNA, 67 bp, Homo sapiens
  source 1..67: mol_type = genomic DNA
cggaccaggggcgtttttagggatcccagtagttctcgtggtgctgcgcggcgatgatgatgactac

SEQ ID NO: 89 - DNA, 65 bp, Homo sapiens
  source 1..65: mol_type = genomic DNA
cggacgggcgcggtgtctcacgcctgtaatcccagcactttgggaggccgaggcgggcggatcac

SEQ ID NO: 90 - DNA, 68 bp, Homo sapiens
  source 1..68: mol_type = genomic DNA
cgccttctctggaaggctccctcatcctctgtcgtagtccagggctccctcctagacctgcggccccg

SEQ ID NO: 91 - DNA, 223 bp, Homo sapiens
  source 1..223: mol_type = genomic DNA
cgccttggcagtgctcgctaagtgtttgcatttttttccctccctgtaaccgctagaccaccacggaacttgcattttttgctactggatgacaggtcttcctcctctcccagggtggctgtctggcaggtttccccacttcctgcagtcttctctgccctaggggaccagtagccatgtttctgccccaacaagtaacctccttgccctgtcctggctcccg

SEQ ID NO: 92 - DNA, 69 bp, Homo sapiens
  source 1..69: mol_type = genomic DNA
cgacgtaggcttcataccctcccttcggaaactcagtccgctgaccaaagccgcagtgttcaggccccg

SEQ ID NO: 93 - DNA, 72 bp, Homo sapiens
  source 1..72: mol_type = genomic DNA
cgcttttcctcttgccgccgcttcgcttctccgcctccgcaggtgacagtgcctggcggccgtagtcccccg

SEQ ID NO: 94 - DNA, 119 bp, Homo sapiens
  source 1..119: mol_type = genomic DNA
cggtgggttcttggtcttgctgacttccagaatgaagccacagacccttgcagtgagtgttatagctcttaaagacggtatgtcctaagtttattccttcagatgttcaggtgtgtccg

SEQ ID NO: 95 - DNA, 64 bp, Homo sapiens
  source 1..64: mol_type = genomic DNA
cgcgagccgcagtgcagggccctccgcggacccattttctcccatcaccaccagggcggcgccg

SEQ ID NO: 96 - DNA, 17 bp, Homo sapiens
  source 1..17: mol_type = genomic DNA
cgctatttttaccgccg

SEQ ID NO: 97 - DNA, 128 bp, Homo sapiens
  source 1..128: mol_type = genomic DNA
cgggaggaacaaacaactccagatgcgccgccttaagaggtgtaacactcaccacgaaggtcggcagcttcactcctgagccagcgaaaccacgaacccaccagaaggaataaactccgaacacatcc

SEQ ID NO: 98 - DNA, 52 bp, Homo sapiens
  source 1..52: mol_type = genomic DNA
cggctaggtgagggcgcgagcgggcgagcgagcgagagtggtgaggggggac

SEQ ID NO: 99 - DNA, 37 bp, Homo sapiens
  source 1..37: mol_type = genomic DNA
cggggcaggatccgctcccgcgattagctctgggagc

SEQ ID NO: 100 - DNA, 41 bp, Homo sapiens
  source 1..41: mol_type = genomic DNA
cgggccagaaatggggacctcagagctccaccgaggcgctc

SEQ ID NO: 101 - DNA, 34 bp, Homo sapiens
  source 1..34: mol_type = genomic DNA
cggacatggtggctcacgcctgtaatcccaacac

SEQ ID NO: 102 - DNA, 80 bp, Homo sapiens
  source 1..80: mol_type = genomic DNA
cggggacgggcaaaggaacgcagctcgtgagtggcccagagagcgggaaccagagcgccccgacggcagcggaagccacc

SEQ ID NO: 103 - DNA, 73 bp, Homo sapiens
  source 1..73: mol_type = genomic DNA
cgcacaactaacgcaatagcctgaggggtttggtaaacagaagcggccccaggagggggtgggattcgccccg

SEQ ID NO: 104 - DNA, 58 bp, Homo sapiens
  source 1..58: mol_type = genomic DNA
cgatggtagagaaggcagacattatcctgcaaaactgccttcagcgatccccaatccg

SEQ ID NO: 105 - DNA, 52 bp, Homo sapiens
  source 1..52: mol_type = genomic DNA
cgtctgaagttttgttctgtttatgcctccaagcgtgttgccgacacatccg

SEQ ID NO: 106 - DNA, 77 bp, Homo sapiens
  source 1..77: mol_type = genomic DNA
cggggcagcgaggcgtccctgtggggctgccctgcggagcggtggggacgcggagaccgcgcgcacgaggaggacgc

SEQ ID NO: 107 - DNA, 69 bp, Homo sapiens
  source 1..69: mol_type = genomic DNA
cgggggaaaaaaaggaaaaaaaaaggtttctccagtccgcgcgcctcaggcgttagaaatagaaggggc

SEQ ID NO: 108 - DNA, 59 bp, Homo sapiens
  source 1..59: mol_type = genomic DNA
cggatctcgacttcagcgaaggtctgggccaccagcatgacggtgttgaggcagacaac

SEQ ID NO: 109 - DNA, 58 bp, Homo sapiens
  source 1..58: mol_type = genomic DNA
cgggcggagatggcgagcttccagtccacaataaataggaaaaacctgagtacacggc

SEQ ID NO: 110 - DNA, 65 bp, Homo sapiens
  source 1..65: mol_type = genomic DNA
cgaccgtttcttcgacaacgcctactgcggtggcggcgacccgcccgccgagcccccctgctccg

SEQ ID NO: 111 - DNA, 16 bp, Homo sapiens
  source 1..16: mol_type = genomic DNA
cgccatgttcaactcg

SEQ ID NO: 112 - DNA, 79 bp, Homo sapiens
  source 1..79: mol_type = genomic DNA
gcctttccctctccttgtttccgaaggagagcctgcctctcgccccgaggttgaacattccagctccttgctctccccg

SEQ ID NO: 113 - DNA, 123 bp, Homo sapiens
  source 1..123: mol_type = genomic DNA
cggacacccccaggaaggccacgttctgaggttagaaagggaaaaaatcagatctcactgaactatgcccgttaagggggaaatgatcccagttttgaaatccatcttcaaagccctgtaagc

SEQ ID NO: 114 - DNA, 29 bp, Homo sapiens
  source 1..29: mol_type = genomic DNA
cggagagcagccaagggcacccccttgcc

SEQ ID NO: 115 - DNA, 62 bp, Homo sapiens
  source 1..62: mol_type = genomic DNA
gcccagctctgggcgccgcttccagctccctttcctatttcgattccagcctccacccgccg

SEQ ID NO: 116 - DNA, 65 bp, Homo sapiens
  source 1..65: mol_type = genomic DNA
gggcagggtccctcctgcagaaaccgctcctgcccgcagcgcgcgcgcttgctgcctcccgcccg

SEQ ID NO: 117 - DNA, 40 bp, Homo sapiens
  source 1..40: mol_type = genomic DNA
cgctgcggctgaccctgcacactcggctatttttacttcc

SEQ ID NO: 118 - DNA, 240 bp, Homo sapiens
  source 1..240: mol_type = genomic DNA
gcttgccctcctcatcatataggttctcaccacaaggagctgaaagaaaaaaatagttttgtctttgctttttacatataatgaaaaggataaatatctcttacatatgtttttcaatacctatttcttttttaatatgattctttcttatttcataagcaatattttttccatgggaaaaaataaactctgtagccccagctactcgggagtctgaggcaggagaatggcgtgaacccg

SEQ ID NO: 119 - DNA, 79 bp, Homo sapiens
  source 1..79: mol_type = genomic DNA
cggtggaccagggcatgtaaaaaagacaccgacacaatggaaaagaaatcctcgaaggtagaacctcgccgcccgcgcc

SEQ ID NO: 120 - DNA, 74 bp, Homo sapiens
  source 1..74: mol_type = genomic DNA
cggcccatgctcttgcaagggcactgcggtttttgcttgggaaacgggtagcaaacagctaagactcccagaac

SEQ ID NO: 121 - DNA, 62 bp, Homo sapiens
  source 1..62: mol_type = genomic DNA
cgctggggccaggcggaggaaagtagctgggagcaagaagggctggcagggccctgagcgcc

SEQ ID NO: 122 - DNA, 141 bp, Homo sapiens
  source 1..141: mol_type = genomic DNA
cggaggaagagaacgcgtgggcccctgctccaagtgccagcgcacgcctggcccagaggtccatcgggctgccaggacaaatgcgtacgcatagacacgtgcacggagcctccgagagagagcgagagaccaagagcgagc

SEQ ID NO: 123 - DNA, 121 bp, Homo sapiens
  source 1..121: mol_type = genomic DNA
cggaagccacggctgacttgtgcagtagaggaagtcgagttcattttattgaatttattctcattttcagtttgaatggccacatgtggcaggcagttattctattgtgcagtgcttgccg

SEQ ID NO: 124 - DNA, 74 bp, Homo sapiens
  source 1..74: mol_type = genomic DNA
gagcggtcagtggctttccgtccttccagggaacctgcccttaggctgctgggcacgccctttcctctctcccg

SEQ ID NO: 125 - DNA, 159 bp, Homo sapiens
  source 1..159: mol_type = genomic DNA
cgccgaggaggaagggagagggagagtgagagtgaggaaggggggagagaagggggaaaaaccagcagctgtcggcctaattcttctaacactctgcttgtggtcatattagaaaaacagattatgcccctcggtgccactcacttatacttgacatac

SEQ ID NO: 126 - DNA, 60 bp, Homo sapiens
  source 1..60: mol_type = genomic DNA
gcttcctgcaccagctgctgatgcgcctggacgacccctttggcttcgactacgccgccg

SEQ ID NO: 127 - DNA, 66 bp, Homo sapiens
  source 1..66: mol_type = genomic DNA
gcctcctgggttcaagcgattctcctgcctcagctcctgagtagctggcgcgcgccaccacgcccg

SEQ ID NO: 128 - DNA, 73 bp, Homo sapiens
  source 1..73: mol_type = genomic DNA
gttgcgggattatttctaaatcagaaaatgtgcggagggagccatttgacaccttttgtggttactgtttccg

SEQ ID NO: 129 - DNA, 338 bp, Homo sapiens
  source 1..338: mol_type = genomic DNA
cggctaacacggtgaaaccccgtctctactaaaaatacagaaaaaaaattagccagccgtggtggcgagtgcctgtagtcccagctacttgggaggctgaggcaggagaatggcgtgaacccgggaggcggagcttgctgtgagccaagatcgcgccactgcactccagcctgggcgacggaacgagactcagtctcaaaaaaaaaaaaaaaaaaatcctggggaagaacctcttattcttatgcaagtggtttctccaccagggagaaaagtttgattactggctgatggagctgaatctcttggcggggaaggggaaggctccagccgttcatggc

SEQ ID NO: 130 - DNA, 136 bp, Homo sapiens
  source 1..136: mol_type = genomic DNA
ggcttgaccgtgaccttggcctcgcaggcacccccatttctcacccccgctctcccgccccgccgtcttctaaattgtctgcgtcgtcggtgaaggaggcttaggctggctgacggcaggagcccgcggcggctcg

SEQ ID NO: 131 - DNA, 66 bp, Homo sapiens
  source 1..66: mol_type = genomic DNA
cgggaaagggagaggacgccccaggaatgacggcgctgagcccctgcggcgggacaggctctgagc

SEQ ID NO: 132 - DNA, 75 bp, Homo sapiens
  source 1..75: mol_type = genomic DNA
cggggatggggggtaggaggagaggggaggccagggctggctggggggtcggggaggctagggcataggcctggc

SEQ ID NO: 133 - DNA, 67 bp, Homo sapiens
  source 1..67: mol_type = genomic DNA
gatggagtctcgctctgtcgcgcatattggaatgcgacggcgcgatctcggctcacggcaacctccg

SEQ ID NO: 134 - DNA, 26 bp, Homo sapiens
  source 1..26: mol_type = genomic DNA
cggcggggtcgtagtgaggtcaaggc

SEQ ID NO: 135 - DNA, 54 bp, Homo sapiens
  source 1..54: mol_type = genomic DNA
gagtcacgggacctcggcagctactggtagccttcccccacttcagagtggccg

SEQ ID NO: 136 - DNA, 73 bp, Homo sapiens
  source 1..73: mol_type = genomic DNA
gcccacagacccgccccttgccttttcttactttccaggccttccctcccgccccgctctttcacccctcccg

SEQ ID NO: 137 - DNA, 89 bp, Homo sapiens
  source 1..89: mol_type = genomic DNA
gtaattttcctgctcaaaacctttttctgactctcccgccccgtgcttcttaaagtcctcacccgcgaggtggattcccgccctgggcg

SEQ ID NO: 138 - DNA, 64 bp, Homo sapiens
  source 1..64: mol_type = genomic DNA
gagtccgtgtcccacagtctgagactcttcttcccctccccttcccgccccgtgaagtggcccg

SEQ ID NO: 139 - DNA, 59 bp, Homo sapiens
  source 1..59: mol_type = genomic DNA
cgggactctcatccgttcggaaacgcacgtgtacccatcatctcacatccctgaggtgc

SEQ ID NO: 140 - DNA, 31 bp, Homo sapiens
  source 1..31: mol_type = genomic DNA
cgaagctgcgcaaacattctgtaaacacggc

SEQ ID NO: 141 - DNA, 31 bp, Homo sapiens
  source 1..31: mol_type = genomic DNA
cggtccacatggttaacacgcacgcaagccc

SEQ ID NO: 142 - DNA, 53 bp, Homo sapiens
  source 1..53: mol_type = genomic DNA
ggactccccatgccaagggctgcagccccgcaacctcgcttctggattcttcg

SEQ ID NO: 143 - DNA, 64 bp, Homo sapiens
  source 1..64: mol_type = genomic DNA
ggcaacagaaacagggctggttcctgccgccctgcatttcagcagtgacgtgttccaggctccg

SEQ ID NO: 144 - DNA, 67 bp, Homo sapiens
  source 1..67: mol_type = genomic DNA
cggtggtgccaactccatgatacagatgagaaaagtgaggcccaggcgaggcaatgggcacgtggac

SEQ ID NO: 145 - DNA, 56 bp, Homo sapiens
  source 1..56: mol_type = genomic DNA
cggagacgcgtccctgccctctcagagttgacagtccagaggcaaaaaggacaatc

SEQ ID NO: 146 - DNA, 144 bp, Homo sapiens
  source 1..144: mol_type = genomic DNA
cgatgtgcgggatggaggcccagagctgttcatccctgcaaccaatgttcacgcaaccaccagggggcgaaaggactctaaccccacacgtagtgagtggttcccacgccacgttccagtaggagaaatgaagttcccggggac

SEQ ID NO: 147 - DNA, 153 bp, Homo sapiens
  source 1..153: mol_type = genomic DNA
gggccctcaagttgtggggcgcccgcgctgctgtgtccagacagcgttccctgagagctccgggaagcgggaagacagccccgggcgtcccgcctttcttctccagaaaacgcacgccccacatcgcactcccccgttcctcctgctccagcg

SEQ ID NO: 148 - DNA, 171 bp, Homo sapiens
  source 1..171: mol_type = genomic DNA
ggatgcactggtcccacaggccgtgcccgagtggagcactgcgaatggggccaagaaattttggcctttctcgccggacctggctgcctccgcgggcctctccgcctaccgcgctcccgccgcggcccgactcccgcgggtctccgcgccgaacccacctggctcctatcg

SEQ ID NO: 149 - DNA, 94 bp, Homo sapiens
  source 1..94: mol_type = genomic DNA
ggctttggcaccgttgggtctttggagcgaagataggacgctggcgaagggacccccaagcgaatccgggatggaggtgatggggccggggccg

SEQ ID NO: 150 - DNA, 60 bp, Homo sapiens
  source 1..60: mol_type = genomic DNA
gcgcgccagcgcctctcaggcctgccgcctgctctcgcacctgctcgccttccccaggcg

SEQ ID NO: 151 - DNA, 228 bp, Homo sapiens
  source 1..228: mol_type = genomic DNA
cgagcccacatcgttggagacgctgcactcgtagctgccgctgctgtcgcgagttacggcgtcgaggcgcagctccgcgtgatccggcgcctcggcggcggcgggaacaacaggcggcggcggcagcagctgccctttgaaacgccacacagccgaggcgatgcgctgggggctgcctcgcagcagcgagcagcgcaggagcacgggccggcccagcgcctggcgcac

SEQ ID NO: 152 - DNA, 96 bp, Homo sapiens
  source 1..96: mol_type = genomic DNA
cgcctcggcctcccaaagtgctggcattacaggcgcgagccgcagtgcagggccctccgcggacccattttctcccatcaccaccagggcggcgcc

SEQ ID NO: 153 - DNA, 201 bp, Homo sapiens
  source 1..201: mol_type = genomic DNA
cggcgagcatgcttggtcaggtggtcgctccgggagaactgcttggggcagagggggcaggagaagcgcttctcgcccgtgtgcgtcctgtagtggcgggccagctcgtcggaacgcgtaaacttcttgtcgcagtcgagccagtcgcaggagaaagggcgctcacccgtgtgggtgcgctggtgggacttgaggtgcgac

SEQ ID NO: 154 - DNA, 82 bp, Homo sapiens
  source 1..82: mol_type = genomic DNA
cggacagggactcgggcgccaaccggcagatgcgctgcgccctctactggcaggtgcacttccggctgcagcgggcctacgc

SEQ ID NO: 155 - DNA, 109 bp, Homo sapiens
  source 1..109: mol_type = genomic DNA
cgacacaatggaaaagaaatcctcgaaggtagaacctcgccgcccgcgccgcgccgcgccgctcagggccgggccccgcgcgcctcgcgccgccgccgcagctcctcgc

SEQ ID NO: 156 - DNA, 159 bp, Homo sapiens
  source 1..159: mol_type = genomic DNA
gctctgccaggcctgctccctcctggacggcctgaaccgcggtcggccccgcctggccatcggcaagggccgccgggggctggacgaggaggcgacgccggggacgcccggggatccggcccgggcccccacttccgagaccgtccccaccttctagcg

(19) United States
(12) Patent Application Publication
(10) Pub. No.: US 2025/0388974 A1
(43) Pub. Date: Dec. 25, 2025

(54) SYSTEMS AND METHODS FOR DETERMINING BIOLOGICAL AGE OF A SUBJECT

(71) Applicant: Coriell Institute for Medical Research, Camden, NJ (US)

(72) Inventors: Jean-Pierre Issa, Philadelphia, PA (US); Jaroslav Jelinek, Philadelphia, PA (US); Jozel Madzo, Glenside, PA (US); Woonbok Chung, Newtown Square, PA (US)

(73) Assignee: Coriell Institute for Medical Research, Camden, NJ (US)

(21) Appl. No.: 19/244,694

(22) Filed: Jun. 20, 2025

Related U.S. Application Data
(60) Provisional application No. 63/662,060, filed on Jun. 20, 2024.

(51) Int. Cl. C12Q 1/6886; C12Q 1/6806

(52) U.S. Cl. CPC C12Q 1/6886; C12Q 1/6806; C12Q 2600/154; C12Q 2600/156

(57) ABSTRACT

Described herein are methods for determining—inter alia—the age of a subject comprising: calculating a probability distribution of three or more nucleic acid target sequences in cells or biological fluids of the subject; calculating the level of DNA methylation and its probabilistic distribution at each of the nucleic acid target sequences; and determining the age of the subject by comparing the probability distribution of allele chaos within the nucleic acid target sequences relative to a control probability distribution to obtain an average Jensen-Shannon distance (JSD) and an average percent methylation for each nucleic acid target sequence.

### CROSS-REFERENCE TO RELATED APPLICATIONS

This application claims the benefit of U.S. Provisional Application No. 63/662,060, which was filed Jun. 20, 2024, is titled “Systems and Methods for Determining Biological Age of a Subject,” and is incorporated herein by reference as if fully set forth.

### SEQUENCE LISTING

The electronic sequence listing filed herewith, titled “COR-002-US-Sequence Listing.xml,” created on Jun. 20, 2025, and having a file size of 146,612 bytes is incorporated herein by reference as if fully set forth.

### BACKGROUND

Aging is the progressive decline in the physiology of an organism with time, and understanding the molecular and cellular hallmarks of aging could lead to the prevention and treatment of age-related diseases. One of the least understood hallmarks of aging is epigenetic alterations. DNA methylation plays an important role in regulating gene expression, and its dysregulation during aging and age-related disease has been well-established. Studies of DNA methylation changes with age have shown that some CpG sites undergo hypomethylation with age, especially at repetitive DNA sequences, which could lead to activation of retrotransposons, which, in turn, cause genomic instability with age; conversely, DNA hypermethylation with age occurs in gene promoter regions located within/near unmethylated CpG islands. This phenomenon of either gaining or losing methylation at different genomic loci is known as methylation drift or age-related DNA methylation drift. Age-related DNA methylation drift is highly conserved across different species, and this drift is inversely proportional to lifespan. Studies have shown that twins living in the same environment acquire distinct age-related epigenetic changes, which indicates that it is a stochastic process rather than a genetic or environmental onc.

Though the phenomenon of age-related epigenetic drift is well documented, there is little direct evidence for its underlying mechanisms. It was theorized that DNA methylation errors accumulate at specific CpG sites during replication in stem cells, which causes epigenetic drift that is then inherited by their daughter cells. DNA methylation alterations have similar patterns in normal aging tissue and in cancer. Because the addition of a methyl group on DNA occurs during DNA replication, the process of methylation drift with age is likely to be linked with stem cell division. There are various software tools that have been proposed to extract DNA methylation information from complex datasets such as whole genome bisulfite sequencing (“WGBS”). Further, there are various biomarker panels designed to estimate DNA methylation age based on microarray technology. These panels are often referred to as “clocks”. However, these clocks do not measure DNA methylation chaos and there is no biomarker panel designed for analysis of DNA methylation chaos.

### SUMMARY

In an aspect, the invention relates to a biomarker panel optimized to measure DNA methylation chaos in biological materials such as blood, saliva, or other materials from which DNA can be recovered. This biomarker panel can be used for the determination of “biological age,” a process that correlates with healthy and unhealthy aging, healthy and unhealthy exposures, and various disease risk or incidence.

In an aspect, the invention relates to a panel of biomarkers that can be used to measure DNA methylation chaos (DMC) in samples derived from biological materials, for example blood or saliva. This reduces to practice a theoretical concept that has heretofore not been realized as a biomarker panel.

In an aspect, the invention relates to an optimized panel of biomarkers that provides a measure of DNA methylation chaos. The biomarkers target 20 genomic loci discovered by deep bioinformatic analysis of Reduced Representation Bisulfite Sequencing (RRBS) data of DNA derived from blood. The characteristics of these genomic loci that make them suitable for DMC analysis include (1) each includes multiple cytosine targets of DNA methylation (range 3-34) and (2) each shows evidence of DMC that increases with age in the reference DNA set obtained from the NINDS public biobank.

In an aspect, the invention relates to a method of measuring DMC. The method comprises first treating DNA with sodium bisulfite. In some embodiment, the treating is accomplished using commercially available kits. This introduces non-natural sequences into DNA that can be used to infer DMC. Bisulfite treated DNA is then amplified by the Polymerase Chain Reaction. The PCR products are then subjected to sequencing using a deep sequencing platform (e.g., Illumina MiSeq). The sequencing results are then analyzed using a bioinformatic pipeline developed herein for this purpose. In some embodiments, the measurement of DMC is accomplished bioinformatically, which can be achieved by different analyses. For example, the method, in some embodiments, includes a Jensen-Shannon Distance (JSD). JSD measures the similarity between two probability distributions, when the JSD values range from 0 to 1. If two distributions are exactly equal, JSD=0. If they do not overlap JSD=1. In turn, the JSD values can be combined with other information (e.g., DNA methylation levels) to derive a “DMC agc,” which can only be measured using the methods herein. In some embodiments, the DMC age is an ultimate deliverable. In some embodiments, DMC age can be used in biological endpoint studies. Examples of biological endpoint studies contemplated include measurement of disease risk or drug activity. Some individuals have DMC ages that are higher than their chronological age, while others have DMC ages lower than their chronological age. The former group is predicted to have a higher incidence of aging diseases and mortality than the average, while the latter group is predicted to be relatively protected from age-related diseases.

In an aspect, the invention relates to a method for determining age of a subject. The method comprises (a) calculating a probability distribution of three or more nucleic acid target sequences in a cell of the subject; (b) calculating a percent methylation at each of the nucleic acid target sequences; (c) determining the age of the subject by comparing the probability distribution of allele methylation within the nucleic acid target sequences relative to a control probability distribution and an average percent methylation for each nucleic acid target sequence.

In some embodiments, the step of calculating the average percent methylation of the three or more nucleic acid target sequences comprises: (a) sequencing DNA in the cell to obtain at least a portion of the nucleic acid sequence of the three or more nucleic acid target sequences; (b) analyzing the at least a portion of the nucleic acid sequence of the three or more nucleic acid target sequences to determine methylation levels at one or a plurality of CpG sites within the three or more nucleic acid target sequences.

In an aspect, the invention relates to a method for determining age of a subject comprising: (a) calculating a probability distribution of three or more nucleic acid target sequences from a sample; (b) calculating a percent methylation and a Jensen-Shannon distance (JSD) at each of the nucleic acid target sequences; (c) determining the age of the subject by comparing the probability distribution of allele methylation within the nucleic acid target sequences relative to a control probability distribution to obtain an average JSD and an average percent methylation for each nucleic acid target sequence.

In some embodiments, the step of calculating the average percent methylation of the three or more nucleic acid target sequences comprises: (a) sequencing DNA in the cell to obtain at least a portion of the nucleic acid sequence of the three or more nucleic acid target sequences; (b) analyzing the at least a portion of the nucleic acid sequence of the three or more nucleic acid target sequences to determine methylation levels at one or a plurality of CpG sites within the three or more nucleic acid target sequences.

In some embodiments, the step of sequencing comprises sequencing using a deep sequencing platform. In some embodiments, the method comprises the step of amplifying DNA from a sample to generate amplified copies of the three or more nucleic acid target sequences, wherein the three or more nucleic acid target sequences comprises a plurality of CpG sites.

In some embodiments, any one of the methods described herein comprises, or further comprises: (i) analyzing amplified copies of the three or more nucleic acid target sequences to determine an individual value of methylation levels at each CpG site at the three or more nucleic acid target sequences; and (ii) calculating an unmethylated CpG average for each of the three or more nucleic acid target sequences.

In some embodiments, the method further comprises calculating epiallele frequencies. In some embodiments, the step of calculating epiallele frequency is calculated from: (i) determining a level of methylation levels at CpG sites across a DNA sample; and (ii) calculating an unmethylated CpG average for each of the three or more nucleic acid target sequences.

In some embodiments, the step of calculating epiallele frequency is further calculated after steps (i) and (ii) by performing steps: (iii) identifying CpGs only within the three or more nucleic acid target sequences; and (iv) counting the number of methylated CpGs within the three or more nucleic acid target sequences.

Differential methylation analysis between two samples can also be performed by quantifying the dissimilarity d between the two distributions of the methylation levels using their Jensen-Shannon distance (JSD), where M is the average PMF of the two probability distributions P and Q. PMF stands for the probability mass function of methylation within each genomic region.

In some embodiments, the average JSD is calculated by the formula:

$$M = \frac{( P + Q )}{2}$$

$$JSD ( P ‖ Q ) = \sqrt{\frac{{D}_{KL} ( P ‖ M ) + {D}_{KL} ( Q ‖ M )}{2}}$$

Wherein M is the mixed distribution of two samples DNA-methylation distributions P and Q, D_{KL} is Kullback-Leibler divergence and JSD is the Jensen-Shannon Distance is the distance of these two epiallele distributions.

In some embodiments, the method further comprises calculating the Kullback-Leibler divergence in methylation by the following formula:

$${D}_{KL} ( P ‖ M ) = {\sum }_{x} P ( x ) {log}_{2} ( \frac{P ( x )}{M ( x )} )$$

which is a method for calculating JSD will yield following formula:

$$JSD ( P ‖ Q ) = \sqrt{\frac{1}{2} {\sum }_{x ϵ Ω} p ( x ) {log}_{2} ( \frac{p ( x )}{p ( x ) + q ( x )} ) + q ( x ) {log}_{2} ( \frac{q ( x )}{p ( x ) + q ( x )} )}$$

wherein, x is the frequency of methylated CpGs on an epiallele P, and Q is the control probability distribution of a control allele; P is the probability distribution of allele chaos of methylation within a first genomic region and Q is the probability distribution of methylation within controls, JSD equals 1 if the probability distributions do not overlap; and if the probability distributions fully overlap, then JSD equals 0; and if probability distribution partially overlap, then JSD is from about 0 to about 1. In some embodiments, Q is the allele frequency of DNA from cells that are from about 1 month to about 12 months of age. In some embodiments, P is the probability distribution of allele frequency in DNA from cell that more than about 12 months of age.

In some embodiments, the method further comprises:
(i) amplifying the DNA to generate the three or more distinct nucleic acid targets;
(ii) analyzing data from the sequenced DNA to determine methylation levels at each CpG site;
(iii) calculating an unmethylated CpG average for each of the three or more nucleic acid target sequences;
(iv) calculating epiallele frequencies from (ii) and (iii);
(v) counting the CpGs within the three or more nucleic acid target sequences;
(vi) counting a number of methylated CpGs in the three or more nucleic acid target sequences;
(vii) calculating methylation chaos by determining the average percent methylation and the average Jensen-Shannon distance (JSD) at three or more nucleic acid target sequences.

In some embodiments, at least one of the three or more nucleic acid target sequences is amplified using one or a pair of primers comprising at least about 75, about 80, about 85, about 90, about 91, about 92, about 93, about 94, about 95, about 96, about 97, about 98, or about 99% sequence identity to the sequences of Table 1, 4, or 5 (below). In some embodiments, at least one of the three or more nucleic acid target sequences is amplified using one or a pair of primers comprising about 100% sequence identity to the sequences of Table 1, 4, or 5 (below). In some embodiments, one of the three or more nucleic acid target sequences is amplified using one or a pair of primers chosen from Table 1, 4, or 5 (below).

In some embodiments, the cell is a cancer cell. In some embodiments, the cell is a stem cell. In some embodiments, the stem cell is an adult stem cell. In some embodiments, the method is free of a step correlating the amount of differentiation of the cell to the age of the cell.

In some embodiments, the disclosure provides a method of determining the chaos of DNA methylation comprising:
(a) calculating a probability distribution of three or more nucleic acid target sequences in a cell of the subject;
(b) calculating a percent methylation and a Jensen-Shannon distance (JSD) at each of the nucleic acid target sequences;
(c) determining the age of the subject by comparing the probability distribution of allele chaos within the nucleic acid target sequences relative to a control probability distribution to obtain an average JSD and an average percent methylation for each nucleic acid target sequence.

In some embodiments, the step of calculating the average percent methylation of the three or more nucleic acid target sequences comprises:
(i) sequencing DNA in the cell to obtain at least a portion of the nucleic acid sequence of the three or more nucleic acid target sequences;
(ii) analyzing nucleic acid target sequences to determine methylation levels at one or a plurality of CpG sites within the three or more nucleic acid target sequences.

In some embodiments, any one of the methods described herein comprises:
(i) analyzing amplified copies of the three or more nucleic acid target sequences to determine an individual value of methylation levels at each CpG site at the three or more nucleic acid target sequences; and
(ii) calculating an unmethylated CpG average for each of the three or more nucleic acid target sequences.

In some embodiments, the method further comprises calculating epiallele frequencies. In some embodiments, the step of calculating epiallele frequency is calculated from: (i) determining an individual value of methylation levels at each CpG site; and (ii) calculating an unmethylated CpG average for each sample.

In some embodiments, the step of calculating epiallele frequency is further calculated after (i) and (ii) by performing steps: (iii) identifying CpGs only within the three or more nucleic acid target sequences; and (iv) counting the number of methylated CpGs within the three or more nucleic acid target sequences.

In some embodiments, the disclosure provides a computer program product encoded on a computer-readable storage medium, wherein the computer program product comprises instructions for:
(a) calculating a probability distribution of three or more nucleic acid target sequences in a cell of the subject;
(b) calculating a percent methylation and a Jensen-Shannon distance (JSD) at each of the nucleic acid target sequences;
(c) determining chaos of DNA methylation by comparing the probability distribution of allele methylation with a control probability distribution to obtain an average percent methylation and an average JSD for each nucleic acid target sequence.

In some embodiments, the computer program product further provides a step of correlating the chaos of DNA methylation with the age of the cell. In some embodiments, the computer program product further comprises instructions for selecting a treatment for the subject based upon the age of the cell. In some embodiments, the computer program product further comprises instructions for: assigning a score to the amount of chaos of DNA methylation; comparing the score to a first threshold; and classifying the subject as being likely to respond to a treatment, if the score exceeds or falls below a first threshold; wherein each of steps (d), (c), and (f) are performed after step (c), and wherein the first threshold is calculated relative to a first control dataset.

In some embodiments, the step (d) is performed by using Levene's test of equal variance and corrected by Bonferroni correction; wherein step (b) further comprises a Jensen-Shannon distance (JSD) at each of the nucleic acid target sequences; and wherein step (c) further comprises determining the chaos of DNA methylation by comparing the probability distribution of allele methylation with a control probability distribution to obtain an average percent methylation and an average JSD for each nucleic acid target sequence.

In some embodiments, the disclosure provides a system comprising the computer program product described above and one or more of: a processor operable to execute programs; and a memory associated with the processor.

In some embodiments, the disclosure provides a system for identifying an age of a cell in a subject, the system comprising: a processor operable to execute programs; a memory associated with the processor; a database associated with said processor and said memory; and a program stored in the memory and executable by the processor, the program being operable for: (i) calculating a probability distribution of three or more nucleic acid target sequences in a cell of the subject; (ii) calculating a percent methylation and a Jensen-Shannon distance (JSD) at each of the nucleic acid target sequences; and (iii) determining chaos of DNA methylation by comparing the probability distribution of allele methylation with a control probability distribution to obtain an average percent methylation and an average JSD for each nucleic acid target sequence.

In some embodiments, the cell is from a sample of the subject. In some embodiments, the cell is a stem cell.

In some embodiments, the disclosure provides a system for identifying the chaos of DNA methylation of DNA in a cell in a subject, the system comprising: a processor operable to execute programs; a memory associated with the processor; a database associated with said processor and said memory; and a program stored in the memory and executable by the processor, the program being operable for: (i) calculating a probability distribution of three or more nucleic acid target sequences in a cell of the subject; (ii) calculating a percent methylation and a Jensen-Shannon distance (JSD) at each of the nucleic acid target sequences; and (iii) determining the chaos of DNA methylation by comparing the probability distribution of allele methylation with a control probability distribution to obtain an average percent methylation and an average JSD for each nucleic acid target sequence. In some embodiments, the cell is from a sample of the subject. In some embodiments, the cell is a stem cell.

The disclosure relates to a computer program product encoded on a computer-readable storage medium comprising instructions for the aforementioned steps of the disclosed algorithm.

The disclosure relates to a computer program product operable in a system or device within a system that applies an algorithm to predict an estimated age.

In some embodiments, the disclosure relates to a kit comprising one or more primer complementary to at least one target sequence. In some embodiments, the at least one target sequence comprises three target sequences. In some embodiments, the at least one target sequence is chosen from Table 1, 4, or 5. In some embodiments, the one or more primer comprises at least one set of amplifying primers, each comprising a forward primer and a reverse primer. In some embodiments, the at least one set of amplifying primers is chosen from one or more matched set of forward and reverse primers capable of amplifying the at least one target sequence. In some embodiments, the at least one set of amplifying primers is chosen from one or more matched set of forward and reverse primers chosen form Table 2. In some embodiments, the at least one set of amplifying primers comprises at least three sets of amplifying primers. In some embodiments, the one or more primer comprises a sequencing primer. In some embodiments, the kit further comprises one or more reagent for bisulfite sequencing. In some embodiments, the kit further comprises instructions for conducting a method of determining an age or estimated age of a subject. In some embodiments, the kit further comprises a computer program product comprising instructions for one or both of (i) sequencing DNA in a cell to obtain at least a portion of nucleic acid sequence of the at least one nucleic acid target sequences; and (ii) analyzing at least a portion of the nucleic acid sequence of the at least one nucleic acid target sequences to determine methylation levels at one or a plurality of CpG sites within the at least one nucleic acid target sequences.

In some embodiments, the disclosure relates to a kit comprising (a) a computer program product comprising instructions for one or both of (i) sequencing DNA in a cell to obtain at least a portion of nucleic acid sequence of at least one nucleic acid target sequences; and (ii) analyzing at least a portion of the nucleic acid sequence of the at least one nucleic acid target sequences to determine methylation levels at one or a plurality of CpG sites within the at least one nucleic acid target sequences; and one or more of: (b) one or more primer complementary to at least one target sequence; and (c) one or more reagent for bisulfite sequencing. In some embodiments, the at least one target sequence comprises three target sequences. In some embodiments, the at least one target sequence is chosen from Table 1, 4, or 5. In some embodiments, the one or more primer comprises at least one set of amplifying primers, each comprising a forward primer and a reverse primer. In some embodiments, the at least one set of amplifying primers is chosen from one or more matched set of forward and reverse primers capable of amplifying the at least one target sequence. In some embodiments, the at least one set of amplifying primers is chosen from one or more matched set of forward and reverse primers chosen form Table 2. In some embodiments, the at least one set of amplifying primers comprises at least three sets of amplifying primers. In some embodiments, the one or more primer comprises a sequencing primer. In some embodiments, the kit comprises the one or more primer complementary to at least one target sequence and the one or more reagent for bisulfite sequencing. In some embodiments, the kit further comprises instructions for conducting a method of determining an age or estimated age of a subject.

In some embodiments, the disclosure relates to a method treating a subject. In some embodiments, the method comprises (a) calculating a probability distribution of three or more nucleic acid target sequences in cells or biological fluids of the subject; (b) calculating a level of DNA methylation probabilistic distribution at each of the nucleic acid target sequences; (c) determining an estimated age of the subject by comparing the probability distribution of allele chaos within the three or more nucleic acid target sequences relative to a control probability distribution to obtain an average percent methylation and an average Jensen-Shannon distance (JSD) for each nucleic acid target sequence; and (d) administering a hypomethylating drug to the subject when the estimate age is greater than the actual age of the subject. In some embodiments, the hypomethylating drug comprises one or more of 5-azacytidine, 5-aza-2′-deoxycytidine, SGI-110, 5-fluro-2′-deoxycytidine, zebularine, CP-4200, RG108, or nanaomycin. In some embodiments, the administering a hypomethylating drug comprises administering a therapeutically effective dose of the hypomethylating drug. In some embodiments, the therapeutically effective dose is about 0.1 mg/kg to about 2.0 mg/kg. In some embodiments, the administering a hypomethylating drug comprises oral, subcutaneous, or intravenous delivery of the hypomethylating drug. In some embodiments, the administering occurs over the course of about 1 to about 10 days.

### BRIEF DESCRIPTION OF THE DRAWINGS

FIG. 1A illustrates average percentage of methylation vs chronological age at 20 target loci. DNA methylation (y-axis) was averaged for all measured CpG sites and plotted against the chronological age of the individuals studied (x-axis). Pearson correlation (r) and p-values (p) are shown for each target. All but one targets show statistically significant correlations with age (p<0.05). See also Table 7.

FIG. 1B illustrates Jensen-Shannon Distance (JSD) vs chronological age at 20 target loci. For each target, JSD (y-axis) was calculated based on the average of two cord blood samples as a control. JSD was plotted against the chronological age of the individuals studied (x-axis). Pearson correlation (r) and p-values (p) are shown for each target. All targets but one show statistically significant correlations with age (p<0.05). See also Table 7.

FIG. 2 illustrates correlation between the predicted methylation age and the chronological age. Chronological age (x-axis) and calculated DMC age (y-axis).

FIG. 3A illustrates average percentage of methylation vs chronological age at 20 target loci in the validation set. Pearson correlation (r) and p-values (p) are shown for each target. See Table 9.

FIG. 3B illustrates JSD vs chronological age at 20 target loci in the validation set. Pearson correlation (r) and p-values (p) are shown for each target.

FIG. 4 illustrates correlation between the predicted and chronological age in the validation set. Chronological age (x-axis) and calculated DMC age (y-axis) in the validation dataset.

FIG. 5A illustrates correlation of JSD between technical PCR replicates.

FIG. 5B illustrates correlation of JSD between replicates from independent DNA bisulfite treatment.

FIG. 6A illustrates predicted vs chronological age in leukemia samples. DMC age acceleration in leukemia. Correlation between chronological age (x-axis) and calculated DMC age (y-axis) in the leukemia dataset.

FIG. 6B illustrates decrease of predicted age after treatment with hypomethylating drugs. DMC age in leukemia decreases after treatment with a hypomethylating drug. DMC acceleration was calculated as a difference between the DMC age and chronological age. DO shows DMC before the treatment, D7 and D14 denote the days of treatment.

FIG. 7 illustrates DNA methylation and JSD are stable across subpopulations of white blood cells. Average DNA methylation (Top) or JSD (bottom) for different white blood cell compartments in six subjects. WB, whole blood; MC, monocytes; GN, granulocytes, B, B cells; NK, natural killer cells; T, T cells.

### DETAILED DESCRIPTION

Various terms relating to the methods and other aspects of the present disclosure are used throughout the specification and claims. Such terms are to be given their ordinary meaning in the art unless otherwise indicated. Other specifically defined terms are to be construed in a manner consistent with the definition provided herein.

As used in this specification and the appended claims, the singular forms “a,” “an,” and “the” include plural referents unless the content clearly dictates otherwise.

The term “more than 2” as used herein is defined as any whole integer greater than the number two, e.g. 3, 4, or 5.

The term “about” as used herein when referring to a measurable; for example, a value an amount, a temporal duration, and the like, is meant to encompass variations of ±20%, ±10%, ±5%, ±1%, ±0.9%, ±0.8%, ±0.7%, ±0.6%, ±0.5%, ±0.4%, ±0.3%, ±0.2% or ±0.1% from the specified value, as such variations are appropriate to perform the disclosed methods.

The phrase “and/or,” as used herein in the specification and in the claims, should be understood to mean “either or both” of the elements so conjoined; i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive; i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, “either,” “one of,” “only one of,” or “exactly one of” “Consisting essentially of,” when used in the claims, shall have its ordinary meaning as used in the field of patent law.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the term “epiallele” means an expressible nucleic acid sequence of a subject that varies due to epigenetic modifications across a population.

As used herein, the phrase “integer from X to Y” means any integer that includes the endpoints. That is, where a range is disclosed, each integer in the range including the endpoints is disclosed. For example, the phrase “integer from X to Y” discloses 1, 2, 3, 4, or 5 as well as the range 1 to 5.

The term “plurality” as used herein is defined as any amount or number greater or more than 1.

As used herein, “substantially equal” can be, for example, within a range known to be correlated to an abnormal or normal range at a given measured metric. For example, if a control sample is from a diseased patient, substantially equal is within an abnormal range. If a control sample is from a patient known not to have the condition being tested, substantially equal is within a normal range for that given metric.

As used herein, the term “animal” includes, but is not limited to, humans and non-human vertebrates such as wild animals, rodents, such as rats, ferrets, and domesticated animals, and farm animals, such as dogs, cats, horses, pigs, cows, sheep, and goats. In some embodiments, the animal is a mammal. In some embodiments, the animal is a human. In some embodiments, the animal is a non-human mammal.

The term “diagnosis” or “prognosis” as used herein refers to the use of information (e.g., genetic information or data from other molecular tests on biological samples, signs and symptoms, physical exam findings, cognitive performance results, etc.) to anticipate the most likely outcomes, timeframes, and/or response to a particular treatment for a given disease, disorder, or condition, based on comparisons with a plurality of individuals or subjects sharing common nucleotide sequences, symptoms, signs, family histories, or other data relevant to consideration of a subject or patient's health status.

As used herein, the phrase “in need thereof” means that the animal or mammal has been identified or suspected as having a need for the particular method or treatment. In some embodiments, the identification can be by any means of diagnosis or observation. In any of the methods and treatments described herein, the animal or mammal can be in need thereof.

As used herein, the term “mammal” means any animal in the class Mammalia such as rodent (i.e., mouse, rat, or guinea pig), monkey, cat, dog, cow, horse, pig, or human. In some embodiments, the mammal is a human. In some embodiments, the mammal refers to any non-human mammal. The present disclosure relates to any of the methods wherein the sample is taken from a mammal or non-human mammal. The present disclosure relates to any of the methods or compositions of matter wherein the sample is taken from a human or non-human primate.

As used herein, the term “predicting” refers to making a finding that an individual or subject of the disclosure has a significantly enhanced probability or likelihood of experiencing a biological response or event. In some embodiments, predicting means making a finding that an individual has a significantly enhanced probability or likelihood of benefiting from and/or responding to an aging treatment. In some embodiments, predicting means estimating an age of a subject by calculating an amount of methylation of at least three nucleic acid sequences in a sample.

As used herein, the term “sample” refers generally to a limited quantity of a substance which is intended to be similar to and represent a larger amount of that substance. In the present disclosure, a sample is a collection, composition comprising fluid, blood, plasma, swab, brushing, scraping, biopsy, removed tissue, or surgical resection that is to be tested. In some embodiments, the sample is bodily fluid such as fluid from a cyst. In some embodiments, the sample comprises a cell or plurality of cells. In some embodiments, samples are taken from a patient or subject that has an unknown age. In some embodiments, the sample comprises cells from a subject. In some embodiments, a sample believed to comprise one or a plurality of cells derived from a subject with an unknown age is compared to a control sample that contains cells from a subject with a known age. As used herein, “control sample” or “reference sample” refer to samples with a known presence, absence, or quantity of substance being measured, that is used for comparison against an experimental sample.

A “score” is a numerical value that may be assigned or generated after normalization of the value based upon the presence, absence, or value of methylation of nucleic acid samples from a subject with an unknown age. In some embodiments, the score is normalized in respect to a control data value or value from a subject or population of subjects with a known age or from a sample free of a cell associated with an aging disorder.

As used herein, the term “stratifying” refers to sorting individuals or subjects into different classes or strata based on the probability of attaining or acquiring an age. In some embodiments, the age is calculated by DNA methylation and, in some embodiments, JSD of a sample from a subject. For example, stratifying a population of individuals with an unknown age involves assigning the individuals into groups based upon on the predicted age.

As used herein, the term “subject,” “individual” or “patient,” used interchangeably, means any animal, including mammals, such as mice, rats, other rodents, rabbits, dogs, cats, swine, cattle, sheep, horses, or primates, such as humans. In some embodiments, the subject is a human. In some embodiments, the subject is a mammal with an unknown age. In some embodiments, the subject is a non-human animal. In some embodiments, the subject is a healthy human being.

As used herein, the term “threshold” refers to a defined value by which a normalized score can be categorized. By comparing to a preset threshold, a subject, with corresponding qualitative and/or quantitative data corresponding to a normalized score, can be classified based upon whether it is above or below the preset threshold.

As used herein, the terms “treat,” “treated,” or “treating” can refer to therapeutic treatment and/or prophylactic or preventative measures wherein the object is to prevent or slow down (lessen) an undesired physiological condition, disorder or disease, or obtain beneficial or desired clinical results. For purposes of the embodiments described herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment can also include eliciting a clinically significant response without excessive levels of side effects. Treatment also includes prolonging survival as compared to expected survival if not receiving treatment. In some embodiments, treatment can lessen the degree to which there is chaos of DNA methylation in a subject. In some embodiments, the treatment reduces methylation of DNA in a subject in need thereof. In some embodiments, the reduction of methylation slows progression of aging.

The term “significantly enhanced” means that the numbers for an observed enhancement within a set of data is unlikely to have happened by chance, normally identified as a p value.

As used herein, the term “therapeutic” means an agent utilized to treat, combat, ameliorate, prevent, or improve an unwanted condition or disease of a patient. In some embodiments, the condition is aging or premature aging.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

As used herein, the term “kit” refers to a set of components provided for purposes of conducting a method herein. In some embodiments, a kit comprises devices or conditions for storage, transport, or delivery of various agents (e.g., oligonucleotides, vectors, drug(s), pharmaceutically acceptable carriers, etc. in appropriate containers) and/or supporting materials (e.g., buffers, written instructions for performing the method etc.) from one location to another. For example, in some embodiments, kits include one or more enclosures (e.g., boxes) containing relevant reaction reagents and/or supporting materials. As used herein, the term “fragmented kit” refers to a kit comprising two or more separate containers that each contain a subportion of total kit components. Containers may be delivered to an intended recipient together or separately. The term “fragmented kit” is intended to encompass kits containing Analyte Specific Reagents (ASR″s) regulated under section 520(e) of the Federal Food, Drug, and Cosmetic Act, but are not limited thereto. Indeed, any delivery system comprising two or more separate containers that each contain a sub-portion of total kit components are included in the term “fragmented kit.” In contrast, a “combined kit” refers to a delivery system containing all components in a single container (e.g., in a single box housing each of the desired components). The term “kit” includes both fragmented and combined kits.

To develop a better understanding of epigenetic mosaicism, the concept of a methylation chaos and information theory was used to quantify age-related DNA methylation drift.

There is no current product that measures DNA methylation chaos.

The invention overcomes the disadvantages of prior art (e.g., DNA methylation “clocks”) that does not measure DNA Methylation Chaos.

The disclosure relates to methods of determining an age of a subject. In some embodiments, the method comprises:
(a) calculating a probability distribution of three or more nucleic acid target sequences in a sample;
(b) calculating a percent methylation at each of the nucleic acid target sequences;
(c) determining the age of the subject by comparing the probability distribution of allele methylation within the nucleic acid target sequences relative to a control probability distribution to obtain an average percent methylation for each nucleic acid target sequence.

In some embodiments, the nucleic acid target sequences are chosen from one or a combination of those nucleic acid sequences from Table 1, 4, or 5. In some embodiments, the nucleic acid target sequences are chosen from one or a combination of functional fragments that comprises about 75%, about 80%, about 85%, about 90%, about 91%, about 92%, about 93%, about 94%, about 95%, about 96%, about 97%, about 98%, or about 99% sequence identity to the nucleic acid sequences from Table 1, 4, or 5. In some embodiments, the nucleic acid target sequences are chosen from one or a combination of functional fragments that comprise 100% sequence identity to the nucleic acid sequences from Table 1, 4, or 5. Unmethylated CpG islands across the human genome are general targets of increased methylation in aging and these can be target sequences herein. In some embodiments, the CpG island target sequences are not associated with canonical gene transcription start sites. In some embodiments, target sequences herein comprise CpG islands at transcription start sites of genes with tissue-specific restricted expression but are from tissues where these genes are not expressed. For example, CpG islands at transcription start sites of brain-specific genes can be affected by aging-related methylation in white blood cells.

In some embodiments, the three or more nucleic acid target sequences comprise B2_165 or a functional fragment thereof. In some embodiments, the nucleic target sequence comprises a CpG island from B2_165.

In some embodiments, the three or more nucleic acid target sequences comprise B3_180 or a functional fragment thereof. In some embodiments, the nucleic target sequence comprises a CpG island from B3_180.

In some embodiments, the three or more nucleic acid target sequences comprise B6_151 or a functional fragment thereof. In some embodiments, the nucleic target sequence comprises a CpG island from B6_151.

In some embodiments, the three or more nucleic acid target sequences comprise B8_175 or a functional fragment thereof. In some embodiments, the nucleic target sequence comprises a CpG island from B8_175.

In some embodiments, the three or more nucleic acid target sequences comprise C13_194 or a functional fragment thereof. In some embodiments, the nucleic target sequence comprises a CpG island from C13_194.

In some embodiments, the three or more nucleic acid target sequences comprise C2_193 or a functional fragment thereof. In some embodiments, the nucleic target sequence comprises CpG sites from C2_193.

In some embodiments, the three or more nucleic acid target sequences comprise Ks02 or a functional fragment thereof. In some embodiments, the nucleic target sequence comprises a CpG island from Ks02.

In some embodiments, the three or more nucleic acid target sequences comprise Ks05 or a functional fragment thereof. In some embodiments, the nucleic target sequence comprises a CpG island from Ks05.

In some embodiments, the three or more nucleic acid target sequences comprise Ks07 or a functional fragment thereof. In some embodiments, the nucleic target sequence comprises a CpG island from Ks07.

In some embodiments, the three or more nucleic acid target sequences comprise Ks08 or a functional fragment thereof. In some embodiments, the nucleic target sequence comprises CpG sites from Ks08.

In some embodiments, the three or more nucleic acid target sequences comprise Ks09 or a functional fragment thereof. In some embodiments, the nucleic target sequence comprises a CpG island from Ks09.

In some embodiments, the three or more nucleic acid target sequences comprise Ks10 or a functional fragment thereof. In some embodiments, the nucleic target sequence comprises a CpG island from Ks10.

In some embodiments, the three or more nucleic acid target sequences comprise Ks11 or a functional fragment thereof. In some embodiments, the nucleic target sequence comprises a CpG island from Ks11.

In some embodiments, the three or more nucleic acid target sequences comprise R23 or a functional fragment thereof. In some embodiments, the nucleic target sequence comprises a CpG island from R23.

In some embodiments, the three or more nucleic acid target sequences comprise R3988 or a functional fragment thereof. In some embodiments, the nucleic target sequence comprises CpG sites from R3988.

In some embodiments, the three or more nucleic acid target sequences comprise R5434 or a functional fragment thereof. In some embodiments, the nucleic target sequence comprises a CpG island from R5434.

In some embodiments, the three or more nucleic acid target sequences comprise R05 or a functional fragment thereof. In some embodiments, the nucleic target sequence comprises CpG sites from R05.

In some embodiments, the three or more nucleic acid target sequences comprise R8436 or a functional fragment thereof. In some embodiments, the nucleic target sequence comprises a CpG island from R8436.

In some embodiments, the three or more nucleic acid target sequences comprise T1_200 or a functional fragment thereof. In some embodiments, the nucleic target sequence comprises a CpG island from T1_200.

In some embodiments, the three or more nucleic acid target sequences comprise T5_275 or a functional fragment thereof. In some embodiments, the nucleic target sequence comprises a CpG island from T5_275.

Some embodiments of the disclosure also relate to methods comprising the steps of:
(a) calculating a probability distribution of three or more nucleic acid target sequences in a sample;
(b) calculating a percent methylation and a Jensen-Shannon distance (JSD) at each of the nucleic acid target sequences;
(c) determining the age of the subject by comparing the probability distribution of allele methylation within the nucleic acid target sequences relative to a control probability distribution to obtain an average JSD and percent methylation for each nucleic acid target sequence.

TABLE 1
Table of chromosomal segment targets identified by start and end sequence components,
human chromosome location, and target nucleic acid sequence length.

| | | | | | | CpG |
|---|---|---|---|---|---|---|
| Target | Chromosome | Start | End | Strand | Length | sites |
| C2_193 | 2 | 236,044,716 | 236,044,906 | top | 191 | 9 |
| Ks05 | 3 | 47,051,176 | 47,051,366 | bottom | 191 | 18 |
| B8_175 | 3 | 51,741,247 | 51,741,421 | bottom | 175 | 15 |
| B3_180 | 3 | 157,812,179 | 157,812,358 | bottom | 180 | 21 |
| B6_151 | 4 | 41,747,818 | 41,747,968 | bottom | 151 | 9 |
| R8436 | 4 | 147,558,398 | 147,558,513 | bottom | 116 | 9 |
| R3988 | 5 | 174,673,908 | 174,674,141 | top | 234 | 3 |
| R05 | 6 | 10,416,394 | 10,416,549 | top | 156 | 8 |
| T5_275 | 6 | 37,616,759 | 37,617,033 | top | 275 | 34 |
| Ks08 | 7 | 15,725,514 | 15,725,691 | bottom | 178 | 15 |
| Ks07 | 7 | 100,231,520 | 100,231,731 | top | 212 | 10 |
| T1_200 | 7 | 130,418,062 | 130,418,261 | top | 200 | 21 |
| Ks09 | 8 | 102,504,781 | 102,504,950 | bottom | 170 | 10 |
| Ks10 | 9 | 1,046,175 | 1,046,301 | top | 127 | 11 |
| Ks11 | 9 | 136,075,447 | 136,075,649 | top | 203 | 18 |
| R23 | 13 | 49,795,399 | 49,795,541 | bottom | 143 | 12 |
| C13_194 | 13 | 53,775,359 | 53,775,489 | top | 131 | 10 |
| R5434 | 15 | 31,775,320 | 31,775,568 | top | 249 | 23 |
| Ks02 | 17 | 40,700,537 | 40,700,703 | bottom | 167 | 18 |
| B2_165 | 19 | 52,104,805 | 52,104,969 | bottom | 165 | 20 |

In an embodiment, the sequence of C2_193 is GTTGTTGTTTGAGGGTATGGAYGATGTGYGGGATGGAGGTTTAGAGTTGTTTATTTT TGTAATTAATGTTTAYGTAATTATTAGGGGGYGAAAGGATTTTAATTTTATAYGTAG TGAGTGGTTTTTAYGTTAYGTTTTAGTAGGAGAAATGAAGTTTTYGGGGAYGGAAG AAGATGGTAGTTATTTGGTGT (SEQ ID NO: 1), or has at least about 75%, about 80%, about 85%, about 86%, about 87%, about 88%, about 89%, about 90%, about 91%, about 92%, about 93%, about 94%, about 95%, about 96%, about 97%, about 98%, about 99% to SEQ ID NO: 1.

In an embodiment, the sequence of Ks05 is GGGTAAATTGAGGTTTTAGTTTAGYGAYGGTTAYGYGGAGGGGGGGYGAGTGGGTT TAGAGGGGGTTAYGGGTTAGGGGAAYGYGAGTTAGGTTAGATTTAGAYGGYGATTT TGGGAYGGTGGTTATGGTAGGTYGAGAYGTTGYGYGYGAAYGTATATTYGGAGAYG GAGTAGTTATAAAATTAGGTTTG (SEQ ID NO: 2), or has at least about 75%, about 80%, about 85%, about 86%, about 87%, about 88%, about 89%, about 90%, about 91%, about 92%, about 93%, about 94%, about 95%, about 96%, about 97%, about 98%, about 99% to SEQ ID NO: 2.

In an embodiment, the sequence of B8_175 is TTAGAGGAYGTTGGAGTAGGAGGAAYGGGGGAGTGYGATGTGGGGYGTGYGTTTTT TGGAGAAGAAAGGYGGGAYGTTYGGGGTTGTTTTTTYGTTTTTYGGAGTTTTTAGGG AAYGTTGTTTGGATATAGTAGYGYGGGYGTTTTATAATTTGAGGGTTYGTAGGATTT TGGGA (SEQ ID NO: 3), or has at least about 75%, about 80%, about 85%, about 86%, about 87%, about 88%, about 89%, about 90%, about 91%, about 92%, about 93%, about 94%, about 95%, about 96%, about 97%, about 98%, about 99% to SEQ ID NO: 3.

In an embodiment, the sequence of B3_180 is GTGYGATAGGAGTTAGGTGGGTTYGGYGYGGAGATTYGYGGGAGTYGGGTYGYGG YGGGAGYGYGGTAGGYGGAGAGGTTYGYGGAGGTAGTTAGGTTYGGYGAGAAAGG TTAAAATTTTTTGGTTTTATTYGTAGTGTTTTATTYGGGTAYGGTTTGTGGGATTAGT GTATTYGGGGAG (SEQ ID NO: 4), or has at least about 75%, about 80%, about 85%, about 86%, about 87%, about 88%, about 89%, about 90%, about 91%, about 92%, about 93%, about 94%, about 95%, about 96%, about 97%, about 98%, about 99% to SEQ ID NO: 4.

In an embodiment, the sequence of B6_151 is GGGTTTTGGATAAGGTTGGGTTTTYGGTTTYGGTTTTATTATTTTTATTTYGGATTYG TTTGGGGGTTTTTTYGTTAGYGTTTTATTTTYGTTTTAAAGATTTAAYGGTGTTAAAG TYGTTTTAGTGAAGAGTAGTATGTTTTGATTTGGA (SEQ ID NO: 5), or has at least about 75%, about 80%, about 85%, about 86%, about 87%, about 88%, about 89%, about 90%, about 91%, about 92%, about 93%, about 94%, about 95%, about 96%, about 97%, about 98%, about 99% to SEQ ID NO: 5.

In an embodiment, the sequence of R8436 is GGGAGTAGGTGTAGGTATTGGGYGTTTGGGGAAGGYGAGTAGGTGYGAGAGTAGG YGGTAGGTTTGAGAGGYGTTGGYGYGYGTTGGGATAAAAATAGAGTGGGAAGG (SEQ ID NO: 6), or has at least about 75%, about 80%, about 85%, about 86%, about 87%, about 88%, about 89%, about 90%, about 91%, about 92%, about 93%, about 94%, about 95%, about 96%, about 97%, about 98%, about 99% to SEQ ID NO: 6.

In an embodiment, the sequence of R3988 is GGTTGAATTTGTATTTTGTATAGAATTTTAAAAAGTTTTTTTGATTGTTGTTTATTTAT TTAAAAAAGTAAAGTATTGTYGGTATTTTTTTGAAAATAAATAATTTAGGTATTYGG TGTTTTTATATGTAATTTATTAATAGTAATGGATAATTTTTTAAAGTTATAAATAGTA TTGGGAGTTYGATTTTAAGAAGTTATTAATTTTAAGAT (SEQ ID NO: 7), or has at least about 75%, about 80%, about 85%, about 86%, about 87%, about 88%, about 89%, about 90%, about 91%, about 92%, about 93%, about 94%, about 95%, about 96%, about 97%, about 98%, about 99% to SEQ ID NO: 7.

In an embodiment, the sequence of R05 is GGGAATYGATTTTTAGTTGTGTTAATTTGTTTTAGTTTTTTTAAGATTTTTTTTTTTAA TTAAAGTAGGGAGAGTTTTTTTATGATTTGGTGATGTTATTAAYGYGGGYGTGTTYG YGAGGTAGAGTTYGGTTGTYGYGGAATTTGGAGGTTTGGG (SEQ ID NO: 8), or has at least about 75%, about 80%, about 85%, about 86%, about 87%, about 88%, about 89%, about 90%, about 91%, about 92%, about 93%, about 94%, about 95%, about 96%, about 97%, about 98%, about 99% to SEQ ID NO: 8.

In an embodiment, the sequence of T5_275 is GGAGATTTGGAAGAGGTAGGTAGTYGAGTTTATATYGTTGGAGAYGTTGTATTYGT AGTTGTYGTTGTTGTYGYGAGTTAYGGYGTYGAGGYGTAGTTTYGYGTGATTYGGY GTTTYGGYGGYGGYGGGAATAATAGGYGGYGGYGGTAGTAGTTGTTTTTTGAAAYG TTATATAGTYGAGGYGATGYGTTGGGGGTTGTTTYGTAGTAGYGAGTAGYGTAGGA GTAYGGGTYGGTTTAGYGTTTGGYGTAYGTTTTGGGAATTGGGTTTTATTT (SEQ ID NO: 9), or has at least about 75%, about 80%, about 85%, about 86%, about 87%, about 88%, about 89%, about 90%, about 91%, about 92%, about 93%, about 94%, about 95%, about 96%, about 97%, about 98%, about 99% to SEQ ID NO: 9.

In an embodiment, the sequence of Ks08 is GATTTTGGAGGGTTTTTAGAGTTGGGGAGTAGTTYGTTYGTTTTGTGTTTTAATTTTT TTAGTTTGGGTTTTAGTATTTYGATTGGGGTYGYGTGYGYGTYGGGGGATTAYGGTY GTTAGGTATTGTTATTTGYGGAGGYGGAGAAGYGAAGYGGYGGTAAGAGGAAAAG CGATAGTT (SEQ ID NO: 10), or has at least about 75%, about 80%, about 85%, about 86%, about 87%, about 88%, about 89%, about 90%, about 91%, about 92%, about 93%, about 94%, about 95%, about 96%, about 97%, about 98%, about 99% to SEQ ID NO: 10.

In an embodiment, the sequence of Ks07 is GAGACGGGTTTTATTATGTTGATTAGGTTGGTTTTAAATTTTTGATTTTAGGTGATTT GTTYGTTTYGGTTTTTTAAAGTGTTGGTATTATAGGYGYGAGTYGTAGTGTAGGGTT TTTYGYGGATTTATTTTTTTTTATTATTATTAGGGYGGYGTYGGAGATTTTTAGGATT

TTATTTTGTTTAAAATTTGATTTTTTATAGTTGGGGGT (SEQ ID NO: 11), or has at least about 75%, about 80%, about 85%, about 86%, about 87%, about 88%, about 89%, about 90%, about 91%, about 92%, about 93%, about 94%, about 95%, about 96%, about 97%, about 98%, about 99% to SEQ ID NO: 11.

In an embodiment, the sequence of T1_200 is TTYGGGAGAATTGTTTGGGGTAGAGGGGGTAGGAGAAGYGTTTTTYGTTYGTGTGY GTTTTGTAGTGGYGGGTTAGTTYGTYGGAAYGYGTAAATTTTTTGTYGTAGTYGAGT TAGTYGTAGGAGAAAGGGYGTTTATTYGTGTGGGTGYGTTGGTGGGATTTGAGGTG YGAYGATTTGTAATAGGTTTTGGTGTAGTTT (SEQ ID NO: 12), or has at least about 75%, about 80%, about 85%, about 86%, about 87%, about 88%, about 89%, about 90%, about 91%, about 92%, about 93%, about 94%, about 95%, about 96%, about 97%, about 98%, about 99% to SEQ ID NO: 12.

In an embodiment, the sequence of is Ks09 GGTGAAATTGGATTTTTAAGTTTGTGTAGGTGAAGGTGTGTGAGAGYGTTYGAGATG GTAGAATAAGAGTTATAGGTAATTTTGTTTTTTYGTTTTTTTTTATTATTTTYGTTYGT TYGTTYGTTYGYGTTTTTATTTAGTYGGAAAGGTGGGATAAGGGGGGGTTTTTT (SEQ ID NO: 13), or has at least about 75%, about 80%, about 85%, about 86%, about 87%, about 88%, about 89%, about 90%, about 91%, about 92%, about 93%, about 94%, about 95%, about 96%, about 97%, about 98%, about 99% to SEQ ID NO: 13.

In an embodiment, the sequence of Ks10 is TGTTAGGTTGGGTTTTTAAGTYGGGTYGTTYGYGTAGAGTTYGGGYGGAGTTGGGG GGTGTGGGGGGAATGTTYGGGGTAGGATTYGTTTTYGYGATTAGTTTTGGGAGYGA AGTGGGAAGGGGTAG (SEQ ID NO: 14), or has at least about 75%, about 80%, about 85%, about 86%, about 87%, about 88%, about 89%, about 90%, about 91%, about 92%, about 93%, about 94%, about 95%, about 96%, about 97%, about 98%, about 99% to SEQ ID NO: 14.

In an embodiment, the sequence of Ks11 is GAAGTAGTATTTGGATTTGAGTTYGGGGAYGGGTAAAGGAAYGTAGTTYGTGAGTG GTTTAGAGAGYGGGAATTAGAGYGTTTYGAYGGTAGYGGAAGTTATYGYGGGYGTT AAATTAGTAAYGYGTTTTTTGAGGATAGGAGGTTAYGGYGTAAAAGTAGATTGGGT TYGGAAATAYGTGTTTTATAAATGGGGAAATGAGT (SEQ ID NO: 15), or has at least about 75%, about 80%, about 85%, about 86%, about 87%, about 88%, about 89%, about 90%, about 91%, about 92%, about 93%, about 94%, about 95%, about 96%, about 97%, about 98%, about 99% to SEQ ID NO: 15.

In an embodiment, the sequence of R23 is GGGGGATGGGAAGTATYGGYGGGTGGAGGTTGGAATYGAAATAGGAAAGGGAGTT GGAAGYGGYGTTTAGAGTTGGGYGAGTAGGGGAAGGGGATTTAGYGTTTGYGYGGT TTTYGGYGGGGYGGATTGTAGGTAGGYGTTTT (SEQ ID NO: 16), or has at least about 75%, about 80%, about 85%, about 86%, about 87%, about 88%, about 89%, about 90%, about 91%, about 92%, about 93%, about 94%, about 95%, about 96%, about 97%, about 98%, about 99% to SEQ ID NO: 16.

In an embodiment, the sequence of C13_194 is GAGAGTTTGGTGGTTTTGGTTAGTATTTYGGATAGGGATTYGGGYGTTAATYGGTAG ATGYGTTGYGTTTTTTATTGGTAGGTGTATTTTYGGTTGTAGYGGGTTTAYGYGGGT AGTTGTTTGGTGGTGAT (SEQ ID NO: 17), or has at least about 75%, about 80%, about 85%, about 86%, about 87%, about 88%, about 89%, about 90%, about 91%, about 92%, about 93%, about 94%, about 95%, about 96%, about 97%, about 98%, about 99% to SEQ ID NO: 17.

In an embodiment, the sequence of R5434 is GGATTAGGGTATGTAAAAAAGATATYGATATAATGGAAAAGAAATTTTYGAAGGTA GAATTTYGTYGTTYGYGTYGYGTYGYGTYGTTTAGGGTYGGGTTTYGYGYGTTTYG YGTYGTYGTYGTAGTTTTTYGYGGTAGTAGTAGGAGTAGTAGTGTTYGGT (SEQ ID NO: 18), or has at least about 75%, about 80%, about 85%, about 86%, about 87%, about 88%, about 89%, about 90%, about 91%, about 92%, about 93%, about 94%, about 95%, about 96%, about 97%, about 98%, about 99% to SEQ ID NO: 18.

In an embodiment, the sequence of Ks02 is TGAGTTTAGGGTTTTTTATTTTATYGGTTTYGTTTTYGGTTTYGGTTTTAGTTTYGGTT TTAGTTTYGGTTTTTGYGGGATYGTYGGYGAATAYGTTTYGGTGTATGGYGGYGGY GTAGTYGAAGTTAAAGGGGTYGTTTAGGYGTATTAGTAGTTGGTGTAGGAAG (SEQ ID NO: 19), or has at least about 75%, about 80%, about 85%, about 86%, about 87%, about 88%, about 89%, about 90%, about 91%, about 92%, about 93%, about 94%, about 95%, about 96%, about 97%, about 98%, about 99% to SEQ ID NO: 19.

In an embodiment, the sequence of B2_165 is GGAGTYGTTAGAAGGTGGGGAYGGTTTYGGAAGTGGGGGTTYGGGTYGGATTTTYG GGYGTTTTYGGYGTYGTTTTTTYGTTTAGTTTTYGGYGGTTTTTGTYGATGGTTAGGY GGGGTYGATYGYGGTTTAGGTYGTTTAGGAGGGAGTAGGTTTGGTAGAGYG (SEQ ID NO: 20), or has at least about 75%, about 80%, about 85%, about 86%, about 87%, about 88%, about 89%, about 90%, about 91%, about 92%, about 93%, about 94%, about 95%, about 96%, about 97%, about 98%, about 99% to SEQ ID NO: 20.

In some embodiments, one or more target nucleic acid sequences in a method, kit, computer program product, or system herein are selected from SEQ ID NOS: 1-20 or a variant thereof have at least about 75%, about 80%, about 85%, about 86%, about 87%, about 88%, about 89%, about 90%, about 91%, about 92%, about 93%, about 94%, about 95%, about 96%, about 97%, about 98%, about 99% to SEQ ID NOS: 1-20. In some embodiments, at least three target nucleic acid sequences in a method, kit, computer program product, or system herein are selected from SEQ ID NOS: 1-20 or a variant thereof have at least about 75%, about 80%, about 85%, about 86%, about 87%, about 88%, about 89%, about 90%, about 91%, about 92%, about 93%, about 94%, about 95%, about 96%, about 97%, about 98%, about 99% to SEQ ID NOS: 1-20. In some embodiments, three target nucleic acid sequences in a method, kit, computer program product, or system herein are selected from SEQ ID NOS: 1-20 or a variant thereof have at least about 75%, about 80%, about 85%, about 86%, about 87%, about 88%, about 89%, about 90%, about 91%, about 92%, about 93%, about 94%, about 95%, about 96%, about 97%, about 98%, about 99% to SEQ ID NOS: 1-20.

In some embodiments, one or more target nucleic acid sequences in a method, kit, computer program product, or system herein are selected from SEQ ID NOS: 61-145 or a variant thereof have at least about 75%, about 80%, about 85%, about 86%, about 87%, about 88%, about 89%, about 90%, about 91%, about 92%, about 93%, about 94%, about 95%, about 96%, about 97%, about 98%, about 99% to SEQ ID NOS: 61-145. In some embodiments, at least three target nucleic acid sequences in a method, kit, computer program product, or system herein are selected from SEQ ID NOS: 61-145 or a variant thereof have at least about 75%, about 80%, about 85%, about 86%, about 87%, about 88%, about 89%, about 90%, about 91%, about 92%, about 93%, about 94%, about 95%, about 96%, about 97%, about 98%, about 99% to SEQ ID NOS: 61-145. In some embodiments, three target nucleic acid sequences in a method, kit, computer program product, or system herein are selected from SEQ ID NOS: 61-145 or a variant thereof have at least about 75%, about 80%, about 85%, about 86%, about 87%, about 88%, about 89%, about 90%, about 91%, about 92%, about 93%, about 94%, about 95%, about 96%, about 97%, about 98%, about 99% to SEQ ID NOS: 61-145.

In some embodiments, one or more target nucleic acid sequences in a method, kit, computer program product, or system herein are selected from SEQ ID NOS: 146-156 or a variant thereof have at least about 75%, about 80%, about 85%, about 86%, about 87%, about 88%, about 89%, about 90%, about 91%, about 92%, about 93%, about 94%, about 95%, about 96%, about 97%, about 98%, about 99% to SEQ ID NOS: 146-156. In some embodiments, at least three target nucleic acid sequences in a method, kit, computer program product, or system herein are selected from SEQ ID NOS: 146-156 or a variant thereof have at least about 75%, about 80%, about 85%, about 86%, about 87%, about 88%, about 89%, about 90%, about 91%, about 92%, about 93%, about 94%, about 95%, about 96%, about 97%, about 98%, about 99% to SEQ ID NOS: 146-156. In some embodiments, three target nucleic acid sequences in a method, kit, computer program product, or system herein are selected from SEQ ID NOS: 146-156 or a variant thereof have at least about 75%, about 80%, about 85%, about 86%, about 87%, about 88%, about 89%, about 90%, about 91%, about 92%, about 93%, about 94%, about 95%, about 96%, about 97%, about 98%, about 99% to SEQ ID NOS: 146-156.

In some embodiments, one or more target nucleic acid sequences in a method, kit, computer program product, or system herein are selected from those at or about at the chromosomal positions of Table 1, Table 4, or Table 5 or a variant thereof have at least about 75%, about 80%, about 85%, about 86%, about 87%, about 88%, about 89%, about 90%, about 91%, about 92%, about 93%, about 94%, about 95%, about 96%, about 97%, about 98%, about 99% to the sequences at or about at the chromosomal positions of Table 1, Table 4, or Table 5. In some embodiments, at least three target nucleic acid sequences in a method, kit, computer program product, or system herein are selected from those at or about at the chromosomal positions of Table 1, Table 4, or Table 5 or a variant thereof have at least about 75%, about 80%, about 85%, about 86%, about 87%, about 88%, about 89%, about 90%, about 91%, about 92%, about 93%, about 94%, about 95%, about 96%, about 97%, about 98%, about 99% to the sequences at or about at the chromosomal positions of Table 1, Table 4, or Table 5. In some embodiments, three target nucleic acid sequences in a method, kit, computer program product, or system herein are selected from those at or about at the chromosomal positions of Table 1, Table 4, or Table 5 or a variant thereof have at least about 75%, about 80%, about 85%, about 86%, about 87%, about 88%, about 89%, about 90%, about 91%, about 92%, about 93%, about 94%, about 95%, about 96%, about 97%, about 98%, about 99% to the sequences at or about at the chromosomal positions of Table 1, Table 4, or Table 5.

Methods of the disclosure include a method of measuring or monitoring DNA methylation in a sample of a subject and methods of measuring toxicity or biological effect of a toxin, drug, therapeutic, biomolecule or pollutant when such molecules, drugs, therapeutics or biomolecules are exposed to a subject or a sample from a subject. In embodiments of monitoring, the methods comprise, at a first time point: (a) calculating a probability distribution of three or more nucleic acid target sequences in a sample; (b) calculating a percent methylation and, optionally, a Jensen-Shannon distance (JSD) at each of the nucleic acid target sequences; (c) determining the age of the subject by comparing the probability distribution of allele methylation within the nucleic acid target sequences relative to a control probability distribution to obtain an average percent methylation and, optionally, an average JSD for each nucleic acid target sequence. In some embodiments, the method of monitoring further comprises repeating steps (a) through (c) at a second time point and (d) comparing the age of the subject at the first and second time points. The method may be a computer-implemented method that calculates the DNA methylation probability distribution over a set of nucleic acids; and optionally, calculating the JSD between the methylated sample and a control sample. In some embodiments, the computer-implemented method relates to a system in which a controller positioned within the device remotely executes software commands to calculate the average JSD and/or the average DNA methylation perform one or more of the following tasks: detect fluorescence from a sample tagged with oligos or primers specific for one or a combination of the nucleic acid sequences tested. In some embodiments, the polymerase reaction is performed by quantitative, semi-quantitative polymerase chain reaction. In such a polymerase reaction, primers complementary to the one, two or three or more nucleic acid sequences chosen for calculation of DNA methylation. In some embodiments, the primers are chosen from one or a combination of any of the primers disclosed in Table 2 (below) or functional sequences or fragments thereof comprising about 75%, about 80%, about 85%, about 86%, about 87%, about 88%, about 89%, about 90%, about 91%, about 92%, about 93%, about 94%, about 95%, about 96%, about 97%, about 98%, about 99% sequence identity to the primers identified in Table 2 (below).

TABLE 2
Primers for bisulfite PCR

| Target | Forward primer (SEQ ID NO.) | Reverse primer (SEQ ID NO.) |
|---|---|---|
| C2_193 | GTTGTTGTTTGAGGGTATGGA (21) | ACACACCAAATAACTACCATCTTCT (41) |
| Ks05 | GGGTAAATTGAGGTTTTAGT (22) | CAAACCTAATTTTATAACTACTCC (42) |
| B8_175 | TTAGAGGAYGTTGGAGTAGGAGGAA (23) | TCCCAAAATCCTACRAACCCTCAAA (43) |
| B3_180 | GTGYGATAGGAGTTAGGTGGGTT (24) | CTCCCCRAATACACTAATCCCACAA (44) |
| B6_151 | GGGTTTTGGATAAGGTTGGGTTTT (25) | TCCAAATCAAAACATACTACTCTTCAC (45) |
| R8436 | GGGAGTAGGTGTAGGTATTGGG (26) | TCCACTCTCCTTCCCACTCT (46) |
| R3988 | GGTTGAATTTGTATTTTGTATAGA (27) | CCAAAAAACTCAATACTCATATATC (47) |
| R05 | GGGAATAGATTTTTAGTTGTGTT (28) | CCCAAACCTCCAAATTC (48) |
| T5_275 | GGAGATTTGGAAGAGGTAGGTAGT (29) | AAATAAAACCCAATTCCCAAAAC (49) |
| Ks08 | GATTTTGGAGGGTTTTTAGA (30) | AACTATCCCTTTTCCTCTTAC (50) |
| Ks07 | YGGGTTTTATTATGTTGATTAGGTTGG (31) | ACAAAACCCCCAACTATAAAAAATCA (51) |
| T1_200 | TTYGGGAGAATTGTTTGGGGTAGAG (32) | RAACTACACCAAAACCTATTACAA (52) |
| Ks09 | GGTGAAATTGGATTTTTAAGT (33) | AAAAAACCCCCCCTTATC (53) |
| Ks10 | TGTTAGGTTGGGTTTTTAAG (34) | CTACCCCTTCCCACTT (54) |
| Ks11 | GAAGTAGTATTTGGATTTGAGTT(35) | ACTCATTTCCCCATTTATA (55) |
| R23 | GGGGGATGGGAAGTAT (36) | AAAACCCCTACCTACAATC (56) |
| C13_194 | GAGAGTTTGGTGGTTTTGGTTAGTA (37) | ATCACCACCAAACAACTACCC (57) |
| R5434 | TGAGTGGYGTTAGTGTAGGTTTAGGT (38) | RAACACTACTACTCCTACTACTAC (58) |
| Ks02 | TGAGTTTAGGGTTTTTTATTTTA (39) | CTTCCTACACCAACTACTAATAC (59) |
| B2_165 | GGAGTYGTTAGAAGGTGGGGA (40) | CRCTCTACCAAACCTACTCCCTCCT (60) |

In some embodiments, methods of the disclosure comprise a method of calculating performed by:
(i) amplifying DNA to generate three or more distinct nucleic acid targets;
(ii) analyzing data from the sequenced DNA to determine methylation levels at each CpG site;
(iii) calculating an unmethylated CpG average for each of the three or more nucleic acid target sequences;
(iv) calculating epiallele frequencies from (ii) and (iii);
(v) counting the CpGs within the three or more nucleic acid target sequences;
(vi) counting a number of methylated CpGs in the three or more nucleic acid target sequences;
(vii) calculating the chaos of DNA methylation by determining the average percent methylation and the average Jensen-Shannon distance (JSD) at the three or more nucleic acid target sequences.

In some embodiments, the step of amplifying comprises isolating nucleic acid molecules from a sample and exposing the nucleic acid molecules to primers chosen from one or a combination of any of the primers disclosed in Table 2 (above) or functional fragments comprising about 75%, about 80%, about 85%, about 86%, about 87%, about 88%, about 89%, about 90%, about 91%, about 92%, about 93%, about 94%, about 95%, about 96%, about 97%, about 98%, about 99% sequence identity to the primers identified in Table 2 (above). In some embodiments, the step of amplifying is performed after a step of converting genomic DNA to cDNA from a sample.

Methods of the disclosure relate to a method of treating a subject in need thereof with an agent. The methods comprise calculating chaos of DNA methylation, estimating an age of subject and treating the subject if the subject's estimated age is highly differentiated from the subject's actual age. In some embodiments, the treating comprises administering a DNA hypomethylating drug to the subject. In some embodiments, the DNA hypomethylating drug is 5-azacytidine, 5-aza-2′-deoxycytidine, SGI-110, 5-fluro-2′-deoxycytidine, zebularine, CP-4200, RG108, or nanaomycin A or a combination of two or more thereof, or pharmaceutically acceptable salts thereof. The administering may comprise administering a therapeutically effective dose of the DNA hypomethylating drug. See Sato T. et al. “DNA Hypomethylating Drugs in Cancer Therapy” (2017) Cold Spring Harb Perspect Med. 7 (5): a026948. doi: 10.1101/cshperspect.a026948. PMID: 28159832; PMCID: PMC5411681, which is incorporated herein by reference as if fully set forth, but where administration here is for treating when the subject's estimated age is highly differentiated from the subject's actual age. See also Griffiths, E. “Oral hypomethylating agents: beyond convenience in MDS” Hematology, ASH Education Program (2021) 2021 (1): 439-447, which is incorporated herein by reference as if fully set forth, but where administration here is for treating when the subject's estimated age is highly differentiated from the subject's actual age. The therapeutically effective dose, in some embodiments, is 0.1 to 2 mg/kg. The therapeutically effective dose, in some embodiments, is about 0.1, about 0.3, about 0.5, about 0.8, about 1.0, about 1.3, about 1.5, about 1.8, or about 2.0 mg/kg. The therapeutically effective dose, in some embodiments, is about 0.1 to about 0.3, about 0.5, about 0.8, about 1.0, about 1.3, about 1.5, about 1.8, or about 2.0 mg/kg. The therapeutically effective dose, in some embodiments, is about 0.3 to about 0.5, about 0.8, about 1.0, about 1.3, about 1.5, about 1.8, or about 2.0 mg/kg. The therapeutically effective dose, in some embodiments, is about 0.5 to about 0.8, about 1.0, about 1.3, about 1.5, about 1.8, or about 2.0 mg/kg. The therapeutically effective dose, in some embodiments, is about 0.8 to about 1.0, about 1.3, about 1.5, about 1.8, or about 2.0 mg/kg. The therapeutically effective dose, in some embodiments, is about 1.0 to about 1.3, about 1.5, about 1.8, or about 2.0 mg/kg. The therapeutically effective dose, in some embodiments, is about 1.3 to about 1.5, about 1.8, or about 2.0 mg/kg. The therapeutically effective dose, in some embodiments, is about 1.5 to about 1.8, or about 2.0 mg/kg. The therapeutically effective dose, in some embodiments, is about 1.8 to about 2.0 mg/kg. The therapeutically effective dose may be administered over the course of 1, 2, 3, 4, 5, 6, 7, 8, 9, or 10 days. The therapeutically effective dose may be administered over the course of 1 to 2, 3, 4, 5, 6, 7, 8, 9, or 10 days. The route of administration may be oral, subcutaneously, or intravenously. The route of administration may be ophthalmic, oral, rectal, vaginal, parenteral, topical, pulmonary, intranasal, buccal, intravenous, intracerebroventricular, intradermal, intramuscular, subcutaneous, intraventricular, intrathecal, intratrachcal, intraperitoneal, in utero delivery, or another route of administration or any combination thereof. The DNA hypomethylating drug may be administered as a pharmaceutical composition. A pharmaceutical composition may include excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other ingredients that may be included in the pharmaceutical compositions of the invention are known in the art and described, for example in Remington's Pharmaceutical Sciences (1985, Genaro, cd., Mack Publishing Co., Easton, PA), which is incorporated herein by reference as if fully set forth. The administration may be as set forth in U.S. Pre-Grant Publication No. 2011/0218170, “Use of 2′-deoxy-4′-thiocytidine and its analogues as dna hypomethylating anticancer agents,” which is incorporated herein by reference as if fully set forth, but applied to any hypomethylating drug, including those described herein. A high DMC age may indicate chronic inflammation and lead to treatments and behavior modifications such as the use of anti-inflammatory drugs, smoking cessation, a calorie restricted dict (including achieving this through the use of GLP1 targeted drugs) and other interventions targeting accelerated aging. In some embodiments, the method comprises administering an anti-inflammatory drug, a smoking cessation treatment, a calorie restricted diet, a GLP1 targeting drug, or an anti-aging treatment.

Percent methylation provides an incomplete picture of DNA methylation changes because it does not consider allelic heterogeneity, also known as methylation entropy. Multiple methods were herein considered to quantify the methylation chaos, including Shannon's entropy and combinatorial entropy. However, those methods fail to consider the directionality of methylation change in the alleles because they treat all completely methylated alleles or all completely unmethylated alleles the same-both have an entropy of zero, which makes methylation entropy change harder to measure. To better quantify the chaos, the change in epiallele distributions was used to calculate the Jensen-Shannon Distance (JSD), where samples are compared to a reference distribution (average JSD in cord blood samples). When the difference in the distance (JSD) between the reference and sample distribution equals or is closer to 0 there is no change in chaos, whereas a JSD of I refers to the greatest distance between reference and sample distribution where there is maximum change in chaos.

The above-described methods can be implemented in a number of ways. For example, the embodiments may be implemented using a computer program product (i.e., software), hardware, software, or a combination thereof. When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device. The computer may be implantable within the subject. Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

A computer employed to implement at least a portion of the functionality described herein may include a memory, coupled to one or more processing units (also referred to herein simply as “processors”), one or more communication interfaces, one or more display units, and one or more user input devices. The memory may include any computer-readable media, and may store computer instructions (also referred to herein as “processor-executable instructions”) for implementing the various functionalities described herein. The processing unit(s) may be used to execute the instructions. The communication interface(s) may be coupled to a wired or wireless network, bus, or other communication means and may therefore allow the computer to transmit communications to and/or receive communications from other devices. The display unit(s) may be provided, for example, to allow a user to view various information in connection with execution of the instructions. The user input device(s) may be provided, for example, to allow a user, a subject or a physician treating the subject to make manual adjustments, make selections, enter data or various other information or parameters, and/or interact in any of a variety of manners with the processor during execution of the instructions. In some embodiments, the parameters include a calculation of DNA methylation, and, optionally, within certain CpG islands of nucleic acid sequences isolated from a subject. In some embodiments, the parameters include any amount assigned to a variable of those algorithms disclosed herein.

The various methods or processes outlined herein may be coded as software that is executable on one or more processors that employ any one of a variety of operating systems or platforms. The disclosure also relates to a as a computer readable storage medium comprising executable instructions to perform any Additionally, such software may be written using any of a number of suitable programming languages and/or programming or scripting tools, and also may be compiled as executable machine language code or intermediate code that is executed on a framework or virtual machine.

In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the disclosure disclosed herein. The computer readable medium or media can be transportable, such that the program or programs stored thereon can be loaded onto one or more different computers or other processors to implement various aspects of the present disclosure as discussed above.

The terms “program” or “software” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present disclosure need not reside on a single computer or processor but may be distributed in a modular fashion amongst a number of different computers or processors to implement various aspects of the present disclosure. Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments. Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Also, the disclosure relates to various embodiments in which one or more computer-readable medium methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

In some embodiments, the disclosure relates to a computer-implemented method of determining DNA methylation chaos. The method comprises: a step of calculating the average percent methylation of the three or more nucleic acid target sequences, which comprises: (i) sequencing DNA in the cell to obtain at least a portion of the nucleic acid sequence of the three or more nucleic acid target sequences; (ii) analyzing at least a portion of the nucleic acid sequence of the three or more nucleic acid target sequences to determine methylation levels at one or a plurality of CpG sites within the three or more nucleic acid target sequences. At least a portion of the steps are performed by a user through a system comprising: (x) a computer program product with instructions for executing the steps (i) through (ii); (y) a processor operable to execute programs; and (2) a memory associated with the processor. In some embodiments, the three or more nucleic acid target sequences are chosen from the nucleic acids identified in Table 1, 4, or 5, or functional sequences or fragments thereof.

In some embodiments, the disclosure relates to a computer-implemented method of determining an age or an estimated age of a subject. The method comprises: a step of calculating the average percent methylation of the three or more nucleic acid target sequences comprises: (i) sequencing DNA in the cell to obtain at least a portion of the nucleic acid sequence of the three or more nucleic acid target sequences; (ii) analyzing at least a portion of the nucleic acid sequence of the three or more nucleic acid target sequences to determine methylation levels at one or a plurality of CpG sites within the three or more nucleic acid target sequences. At least a portion of the steps are performed by a user through a system comprising: (x) a computer program product with instructions for executing the steps (i) through (ii); (y) a processor operable to execute programs; and (z) a memory associated with the processor; and wherein, if the average DNA methylation chaos of the subject is higher than a first threshold, then the subject is characterized as having an aging abnormality.

In some embodiments, the disclosure relates to a system that comprises at least one processor, a program storage (for example, a memory) for storing program code executable on the processor, and one or more input/output devices and/or interfaces, such as data communication and/or peripheral devices and/or interfaces. In some embodiments, the user device and computer system or systems are communicably connected by a data communication network (for example, a Local Area Network (LAN), the Internet, or others), which may also be connected to a number of other client and/or server computer systems. The user device and client and/or server computer systems may further include appropriate operating system software.

In some embodiments, components and/or units of the devices described herein may be able to interact through one or more communication channels or mediums or links; for example, a shared access medium, a global communication network, the Internet, the World Wide Web, a wired network, a wireless network, a combination of one or more wired networks and/or one or more wireless networks, one or more communication networks, an a-synchronic or asynchronous wireless network, a synchronic wireless network, a managed wireless network, a non-managed wireless network, a burstable wireless network, a non-burstable wireless network, a scheduled wireless network, a non-scheduled wireless network, or others.

Discussions herein utilizing terms such as, for example, “processing,” “computing,” “calculating,” “determining,” or the like, may refer to operation(s) and/or process(es) of a computer, a computing platform, a computing system, or other electronic computing device, that manipulate and/or transform data represented as physical (e.g., electronic) quantities within the computer's registers and/or memories into other data similarly represented as physical quantities within the computer's registers and/or memories or other information storage medium that may store instructions to perform operations and/or processes.

Some embodiments may take the form of an entirely hardware embodiment, an entirely software embodiment, or an embodiment including both hardware and software elements. Some embodiments may be implemented in software, which includes but is not limited to firmware, resident software, microcode, or the like.

Furthermore, some embodiments may take the form of a computer program product accessible from a computer-usable or computer-readable medium providing program code for use by or in connection with a computer or any instruction execution system. For example, a computer-usable or computer-readable medium may be or may include any apparatus that can contain, store, communicate, propagate, or transport the program for use by or in connection with the instruction execution system, apparatus, or device disclosed herein.

In some embodiments, the medium may be or may include an electronic, magnetic, optical, electromagnetic, InfraRed (IR), or semiconductor system (or apparatus or device) or a propagation medium. Some demonstrative examples of a computer-readable medium may include a semiconductor or solid state memory, magnetic tape, a removable computer diskette, a Random Access Memory (RAM), a Read-Only Memory (ROM), a rigid magnetic disk, an optical disk, or the like. Some demonstrative examples of optical disks include Compact Disk-Read-Only Memory (CD-ROM), Compact Disk-Read/Write (CD-R/W), DVD, or others.

In some embodiments, a data processing system suitable for storing and/or executing program code may include at least one processor coupled directly or indirectly to memory elements, for example, through a system bus. The memory elements may include, for example, local memory employed during actual execution of the program code, bulk storage, and cache memories which may provide temporary storage of at least some program code in order to reduce the number of times code must be retrieved from bulk storage during execution.

In some embodiments, input/output or I/O devices (including but not limited to keyboards, displays, pointing devices, etc.) may be coupled to the system either directly or through intervening I/O controllers. In some embodiments, network adapters may be coupled to the system to enable the data processing system to become coupled to other data processing systems or remote printers or storage devices, for example, through intervening private or public networks. In some embodiments, modems, cable modems and Ethernet cards are demonstrative examples of types of network adapters. Other suitable components may be used.

Some embodiments may be implemented by software, by hardware, or by any combination of software and/or hardware as may be suitable for specific applications or in accordance with specific design requirements. Some embodiments may include units and/or sub-units, which may be separate of each other or combined together, in whole or in part, and may be implemented using specific, multi-purpose or general processors or controllers. Some embodiments may include buffers, registers, stacks, storage units and/or memory units, for temporary or long-term storage of data or in order to facilitate the operation of particular implementations.

Some embodiments may be implemented, for example, using a machine-readable medium or article which may store an instruction or a set of instructions that, if executed by a machine, cause the machine to perform a method steps and/or operations described herein. Such machine may include, for example, any suitable processing platform, computing platform, computing device, processing device, electronic device, electronic system, computing system, processing system, computer, processor, or the like, and may be implemented using any suitable combination of hardware and/or software. The machine-readable medium or article may include, for example, any suitable type of memory unit, memory device, memory article, memory medium, storage device, storage article, storage medium and/or storage unit; for example, memory, removable or non-removable media, erasable or non-erasable media, writeable or re-writeable media, digital or analog media, hard disk drive, floppy disk, Compact Disk Read Only Memory (CD-ROM), Compact Disk Recordable (CD-R), Compact Disk Re-Writeable (CD-RW), optical disk, magnetic media, various types of Digital Versatile Disks (DVDs), a tape, a cassette, or the like. The instructions may include any suitable type of code, for example, source code, compiled code, interpreted code, executable code, static code, dynamic code, or the like, and may be implemented using any suitable high-level, low-level, object-oriented, visual, compiled and/or interpreted programming language, e.g., C, C++, Java™, BASIC, Pascal, Fortran, Cobol, assembly language, machine code, or the like.

Many of the functional units described in this specification be labeled as circuits in order to more particularly emphasize their implementation independence. For example, a circuit may be implemented as a hardware circuit comprising custom very-large-scale integration (VLSI) circuits or gate arrays, off-the-shelf semiconductors such as logic chips, transistors, or other discrete components. A circuit may also be implemented in programmable hardware devices such as field programmable gate arrays, programmable array logic, programmable logic devices or others.

In some embodiment, the circuits may also be implemented in machine-readable medium for execution by various types of processors. An identified circuit of executable code may, for instance, comprise one or more physical or logical blocks of computer instructions, which may, for instance, be organized as an object, procedure, or function. Nevertheless, the executables of an identified circuit need not be physically located together, but may comprise disparate instructions stored in different locations which, when joined logically together, comprise the circuit and achieve the stated purpose for the circuit. Indeed, a circuit of computer readable program code may be a single instruction, or many instructions, and may even be distributed over several different code segments, among different programs, and across several memory devices. Similarly, operational data may be identified and illustrated herein within circuits, and may be embodied in any suitable form and organized within any suitable type of data structure. The operational data may be collected as a single data set, or may be distributed over different locations including over different storage devices, and may exist, at least partially, merely as electronic signals on a system or network.

The computer readable medium (also referred to herein as machine-readable media or machine-readable content) may be a tangible computer readable storage medium storing the computer readable program code. The computer readable storage medium may be, for example, but not limited to, an electronic, magnetic, optical, electromagnetic, infrared, holographic, micromechanical, or semiconductor system, apparatus, or device, or any suitable combination of the foregoing. As alluded to above, examples of the computer readable storage medium may include but are not limited to a portable computer diskette, a hard disk, a random access memory (RAM), a read-only memory (ROM), an erasable programmable read-only memory (EPROM or Flash memory), a portable compact disc read-only memory (CD-ROM), a digital versatile disc (DVD), an optical storage device, a magnetic storage device, a holographic storage medium, a micromechanical storage device, or any suitable combination of the foregoing. In the context of this document, a computer readable storage medium may be any tangible medium that can contain, and/or store computer readable program code for use by and/or in connection with an instruction execution system, apparatus, or device.

The computer readable medium may also be a computer readable signal medium. A computer readable signal medium may include a propagated data signal with computer readable program code embodied therein, for example, in baseband or as part of a carrier wave. Such a propagated signal may take any of a variety of forms, including, but not limited to, electrical, electro-magnetic, magnetic, optical, or any suitable combination thereof. A computer readable signal medium may be any computer readable medium that is not a computer readable storage medium and that can communicate, propagate, or transport computer readable program code for use by or in connection with an instruction execution system, apparatus, or device. As also alluded to above, computer readable program code embodied on a computer readable signal medium may be transmitted using any appropriate medium, including but not limited to wireless, wireline, optical fiber cable, Radio Frequency (RF), or the like, or any suitable combination of the foregoing.

In one embodiment, the computer readable medium may comprise a combination of one or more computer readable storage mediums and one or more computer readable signal mediums. For example, computer readable program code may be both propagated as an electro-magnetic signal through a fiber optic cable for execution by a processor and stored on RAM storage device for execution by the processor.

Computer readable program code for carrying out operations for aspects of the present disclosure may be written in any combination of one or more programming languages, including an object-oriented programming language, for example, Java, Smalltalk, C++ or the like and conventional procedural programming languages, for example, the “C” programming language or similar programming languages. The computer readable program code may execute entirely on a user's computer, partly on the user's computer, as a stand-alone computer-readable package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The program code may also be stored in a computer readable medium that can direct a computer, other programmable data processing apparatus, or other devices to function in a particular manner, such that the instructions stored in the computer readable medium produce an article of manufacture including instructions which implement the function/act specified in the schematic flowchart diagrams and/or schematic block diagrams block or blocks. Functions, operations, components and/or features described herein with reference to one or more embodiments, may be combined with, or may be utilized in combination with, one or more other functions, operations, components and/or features described herein with reference to one or more other embodiments, or vice versa.

The disclosure relates to a computer program product integrated into or in electrical communication with a controller and a device disclosed herein. The device comprises at least on set of instructions, the instructions comprising steps: (a) calculating a probability distribution of three or more nucleic acid target sequences in a sample; (b) calculating a percent methylation and, optionally, a Jensen-Shannon distance (JSD) at each of the nucleic acid target sequences; (c) determining the age of the subject by comparing the probability distribution of allele chaos within the nucleic acid target sequences relative to a control probability distribution to obtain an average percent methylation and, optionally, an average JSD for each nucleic acid target sequence.

In some embodiments, the disclosure relates to a computer program product encoded on a computer-readable storage medium. The computer program product comprises instructions for: (a) calculating a probability distribution of three or more nucleic acid target sequences in a cell of the subject; (b) calculating a percent methylation and a Jensen-Shannon distance (JSD) at each of the nucleic acid target sequences; (c) determining chaos of DNA methylation by comparing the probability distribution of allele chaos with a control probability distribution to obtain an average percent methylation and an average JSD for each nucleic acid target sequence.

In some embodiments, the methods further comprise a step of correlating the chaos of DNA methylation with the age of the cell. In some embodiments, the method further comprises instructions for selecting a treatment for the subject based upon the age of the cell.

In some embodiments, computer implemented methods of the disclosure further comprise instructions for: (d) assigning a score to the amount of chaos of DNA methylation; (e) comparing the score to a first threshold; and (f) classifying the subject as being likely to respond to a treatment, if the score exceeds or falls below a first threshold; wherein each of steps (d), (c), and (f) are performed after step (c), and wherein the first threshold is calculated relative to a first control dataset.

In some embodiments, step (d) is performed by using Levene's test of equal variance and corrected by Bonferroni correction; wherein step (b) further comprises a Jensen-Shannon distance (JSD) at each of the nucleic acid target sequences; and wherein step (c) further comprises determining chaos of DNA methylation by comparing the probability distribution of allele chaos with a control probability distribution to obtain an average percent methylation and an average JSD for each nucleic acid target sequence.

In some embodiments, the disclosure relates to a system comprising a controller. The controller is operably and electrically linked to one or a combination of: a display, a charging chip, a Bluetooth communication device, each component in operable communication with a computer program product with instructions for executing steps. The steps include (a) calculating a probability distribution of three or more nucleic acid target sequences in a sample; (b) calculating a percent methylation and, optionally, a Jensen-Shannon distance (JSD) at target nucleic acid sequences, which may be each of the nucleic acid target sequences in a method herein, which may be at least three nucleic acid target sequences in a method herein; (c) determining the age of the subject by comparing the probability distribution of allele chaos within the nucleic acid target sequences relative to a control probability distribution to obtain an average percent methylation and, optionally, an average JSD for each nucleic acid target sequence.

In some embodiments, the device further comprises a clock, display, Bluetooth connector and a rechargeable battery source. In some embodiments, the computer program product is operably connected to the device by a remote network, such as a Bluetooth network. In such cases, a software user, such a physician may input values for variable components of operation of the device remotely, and the device may still operate with those instructions.

In some embodiments, the disclosure relates to kit for determining an age or estimated age of a subject. The kit may comprise one or more reagent for a method of determining an age or estimated age of a subject. In some embodiments, the method comprises The method comprises: a step of calculating the average percent methylation of the three or more nucleic acid target sequences comprises: (i) sequencing DNA in the cell to obtain at least a portion of the nucleic acid sequence of the three or more nucleic acid target sequences; (ii) analyzing at least a portion of the nucleic acid sequence of the three or more nucleic acid target sequences to determine methylation levels at one or a plurality of CpG sites within the three or more nucleic acid target sequences. The kit may comprise one or more sequencing primer. The kit may comprise one or more sequencing primer for the step of (i) sequencing DNA in the cell to obtain at least a portion of the nucleic acid sequence of the three or more nucleic acid target sequences. In some embodiments, the one or more sequencing primer are chosen from Table 2. In some embodiments, the kit comprises primers for amplifying a target sequence. In some embodiments, the primers comprise sets of primers that target three or more target sequences. The target sequences, in some embodiments, are chosen from any set forth herein. In some embodiments, the one or more primers comprise one or more matched set of forward and reverse primers in Table 2. In some embodiments, the one or more primers comprise three or more matched sets of forward and reverse primers in Table 2. In some embodiments, the one or more primers comprise three matched sets of forward and reverse primers in Table 2. In some embodiments, the kit comprises one or more reagents for bisulfite sequencing/PCR as set forth herein. In some embodiments, the kit comprises one or more reagents for reduced representation bisulfite sequencing. In some embodiments, the kit comprise instructions for conduction a portion or all of a method of determining an age or estimated age of a subject herein. In some embodiments, the kit comprises a computer program product (software) saved to a memory device. In some embodiments, the kit comprises access to a computer program product or scripts available on another device. The access may be through password or otherwise restricted access to the other device. The other device may be accessed through the Internet; e.g. through a cloud network. The kit may include instructions to access the other device. The kit may include a password or other authentication means to access the other device. In some embodiments, the computer program product comprises instructions for conducting a method herein. In some embodiments, the computer program product comprises instructions for an analysis herein. In some embodiments, the computer program product comprises instructions for a calculation herein. In some embodiments, the computer program product comprises instructions for steps (i) and/or (ii): (i) sequencing DNA in the cell to obtain at least a portion of the nucleic acid sequence of the three or more nucleic acid target sequences; (ii) analyzing at least a portion of the nucleic acid sequence of the three or more nucleic acid target sequences to determine methylation levels at one or a plurality of CpG sites within the three or more nucleic acid target sequences. In some embodiments, a kit herein comprises a therapeutic agent for delivery to a subject when the subject is determined to have an DMC age greater than actual age. The therapeutic agent may be an anti-inflammatory drug, a smoking cessation treatment, a calorie restricted diet, a GLP1 targeting drug, or an anti-aging treatment, a DNA hypomethylating drug, 5-azacytidine, 5-aza-2′-deoxycytidine, SGI-110, 5-fluro-2′-deoxycytidine, zebularine, CP-4200, RG108, or nanaomycin A or a combination of two or more thereof.

Embodiment List—The following list of particular embodiments herein is not limiting to embodiments otherwise described herein.
1. A method for determining age of a subject comprising: (a) calculating a probability distribution of three or more nucleic acid target sequences in cells or biological fluids of the subject; (b) calculating the level of DNA methylation its probabilistic distribution at each of the nucleic acid target sequences; (c) determining the age of the subject by comparing the probability distribution of allele chaos within the three or more nucleic acid target sequences relative to a control probability distribution to obtain an average percent methylation and an average Jensen-Shannon distance (JSD) for each nucleic acid target sequence.
2. The method according to embodiment 1, wherein the step of calculating the average percent methylation of the three or more nucleic acid target sequences comprises: (i) sequencing DNA in the cells or biological fluids to obtain at least a portion of the three or more nucleic acid target sequences; (ii) analyzing the at least a portion of the three or more target sequences to determine methylation levels at one or a plurality of CpG sites within the three or more nucleic acid target sequences.
3. The method according to embodiment 2, wherein the step of sequencing comprises deep sequencing platform sequencing.
4. The method according to any foregoing embodiment, wherein the step (a) comprises: amplifying DNA from the cells or biological fluids to generate amplified copies of the three or more nucleic acid target sequences, wherein the three or more nucleic acid target sequences comprise a plurality of CpG sites.
5. The method according to embodiment 4, wherein step (a) and/or (b) comprises:
(i) analyzing the amplified copies of the three or more nucleic acid target sequences to determine an individual value of methylation levels at each CpG site at the three or more nucleic acid target sequences; and (ii) calculating an unmethylated CpG average for each of the three or more nucleic acid target sequences.
6. The method according to any foregoing embodiment, further comprising calculating epiallele frequencies.
7. The method according to embodiment 6, wherein the step of calculating epiallele frequency is calculated by: (i) determining a level of methylation levels at CpG site across DNA in the cells or biological fluid; and (ii) calculating an unmethylated CpG average for each of the three or more nucleic acid target sequences.
8. The method according to embodiment 7, wherein the step of calculating epiallele frequency is further calculated after steps (i) and (ii) of embodiment 7, by performing steps: (iii) identifying CpGs only within the three or more nucleic acid target sequences; and (iv) counting the number of methylated CpGs within the three or more nucleic acid target sequences.
9. The method according to any foregoing embodiment, wherein the average JSD is calculated by the formulas:

$$\frac{1}{n} {\sum }_{i = 1}^{n} {( JSD )}_{i}$$

$$M = \frac{( P + Q )}{2}$$

$$JSD ( P ‖ Q ) = \sqrt{\frac{{D}_{KL} ( P ‖ M ) + {D}_{KL} ( Q ‖ M )}{2}}$$

wherein JSD is the Jensen-Shannon Distance for a nucleic acid target sequence and n is the number of epialleles.
10. The method according to any foregoing embodiment, further comprising calculating methylation chaos.
11. The method according to embodiment 10, wherein the Jensen-Shannon distance of methylation is calculated by:

$$\frac{1}{n} {\sum }_{i = 1}^{n} {( JSD )}_{i}$$

$$d = \sqrt{\frac{D ( {P}_{L}^{( 1 )} , Q ) + D ( {P}_{L}^{( 2 )} , Q )}{2}}$$

$$D ( P , Q ) = {\sum }_{ℓ} P ( ℓ ) {log}_{2} ( \frac{P ( ℓ )}{Q ( ℓ )} )$$

wherein, [symbol] is the frequency of methylated CpGs on an epiallele, Q is the control probability distribution of a control allele; P is the probability distribution of allele chaos; P_{L}⁽¹⁾ is the probability mass function (PMF) of methylation within a first genomic region and P_{L}⁽²⁾ is the PMF of methylation within a second non-overlapping genomic region and D is the relative entropy, wherein, if the reference and sample probability distributions do not overlap, JSD equals 1; and if the probability distributions fully overlap, then JSD equals 0; and if probability distributions partially overlap, then JSD is from about 0.1 to about 0.9.]
12. The method according to embodiment 11, wherein Q is the allele frequency of DNA from cells that are from about 1 month to about 12 months of age.
13. The method according to embodiment 11, wherein P is the probability distribution of allele frequency in DNA from a cell that is more than about 12 months of age.
14. The method according to embodiment 1, further comprising: (i) amplifying DNA from the cells or biological fluids to generate the three or more nucleic acid target sequences to produce amplified DNA and sequencing the amplified DNA to produce sequence data; (ii) analyzing the sequence data to determine methylation levels at each CpG site; (iii) calculating an unmethylated CpG average for each of the three or more nucleic acid target sequences; (iv) calculating epiallele frequencies from (ii) and (iii); (v) counting the CpGs within the three or more nucleic acid target sequences; (vi) counting a number of methylated CpGs in the three or more nucleic acid target sequences; (vii) calculating methylation chaos by determining the average percent methylation and the average Jensen-Shannon distance (JSD) at the three or more nucleic acid target sequences.
15. The method according to any foregoing embodiment, wherein the amplifying DNA comprises amplifiying at least one of the three or more nucleic acid target sequences with primers comprising one or a pair of primers comprising at least about 75% sequence identity to a sequence in Table 1, 4, or 5, optionally one or a pair of primers comprising a sequence in Table 1.
16. The method according to embodiment 14 or embodiment 15, wherein at least a portion of the DNA is treated with sodium bisulfite prior to being amplified.
17. The method according to embodiment 16, wherein the sulfite treated DNA is amplified by the Polymerase Chain Reaction, and optionally wherein the analyzing comprises comparison of the sequence data to non-bisulfite sequence information, further optionally wherein the non-bisulfite sequence information is obtained from one or both of archived genome sequence information or sequencing of amplified, untreated DNA from the cells or biological fluids.
18. The method according to any foregoing embodiment, wherein the cells are cancer cells.
19. The method according to any one of embodiments 1 to 17, wherein the cells are stem cells.
20. The method according to embodiment 19, wherein the stem cells are adult stem cells.
21. The method according to any foregoing embodiment, wherein the method is free of a step of correlating the amount of differentiation of the cell to the age of the cell.
22. A method of determining chaos of DNA methylation comprising: (a) calculating a probability distribution of three or more nucleic acid target sequences in a cell of the subject; (b) calculating a percent methylation and a Jensen-Shannon distance (JSD) at each of the nucleic acid target sequences; (c) determining a methylation age of the subject by comparing the probability distribution of allele chaos within the nucleic acid target sequences relative to a control probability distribution to obtain an average percent methylation and an average JSD for each nucleic acid target sequence.
23. The method according to embodiment 22, wherein the step of obtaining the average percent methylation for each nucleic acid target sequences comprises: (i) sequencing DNA in the cell to obtain at least a partial nucleic acid sequence of each of the nucleic acid target sequences; (ii) analyzing the at least a partial nucleic acid sequence of each of the nucleic acid target sequences to determine methylation levels at one or a plurality of CpG sites within the three or more nucleic acid target sequences.
24. The method according to embodiment 23, wherein the step of sequencing comprises deep sequencing platform sequencing.
25. The method according to any one of embodiments 22 through 24, wherein the step (a) comprises: amplifying DNA from a sample comprising the cell to generate amplified copies of the three or more nucleic acid target sequences, wherein the three or more nucleic acid target sequences comprises a plurality of CpG sites.
26. The method according to embodiment 22, wherein step (a) and/or (b) comprises: (i) analyzing amplified copies of the three or more nucleic acid target sequences to determine an individual value of methylation levels at each CpG site at the three or more nucleic acid target sequences; and (ii) calculating an unmethylated CpG average for each of the three or more nucleic acid target sequences.
27. The method according to any one of embodiments 22 through 26, further comprising calculating epiallele frequencies.
28. The method according to embodiment 27, wherein the step of calculating epiallele frequency is calculated from: (i) determining an individual value of methylation levels at each CpG site; and (ii) calculating an unmethylated CpG average for each sample.
29. The method according to embodiment 28, wherein the step of calculating epiallele frequency is further calculated after (i) and (ii) of embodiment 7, by performing steps: (iii) identifying CpGs only within the three or more nucleic acid target sequences; and (iv) counting the number of methylated CpGs within the three or more nucleic acid target sequences.
30. The method according to any one of embodiments 22 to 29, wherein the average JSD is calculated by the formulas:

$$\frac{1}{n} {\sum }_{i = 1}^{n} {( JSD )}_{i}$$

$$M = \frac{( P + Q )}{2}$$

$$JSD ( P ‖ Q ) = \sqrt{\frac{{D}_{KL} ( P ‖ M ) + {D}_{KL} ( Q ‖ M )}{2}}$$

wherein JSD is the Jensen-Shannon Distance for a nucleic acid target sequence and n is the number of epialleles.
31. The method according to any one of embodiments 22 through 30, wherein the Jensen-Shannon distance of methylation chaos is calculated by formula:

$$d = \sqrt{\frac{D ( {P}_{L}^{( 1 )} , Q ) + D ( {P}_{L}^{( 2 )} , Q )}{2}}$$

$$D ( P , Q ) = {\sum }_{ℓ} P ( ℓ ) {log}_{2} ( \frac{P ( ℓ )}{Q ( ℓ )} )$$

wherein, [symbol] is the frequency of methylated CpGs on an epiallele, Q is the control probability distribution of a control allele; P is the probability distribution of allele chaos; P_{L}⁽¹⁾ is the probability mass function (PMF) of methylation within a first genomic region and P_{L}⁽²⁾ is the PMF of methylation within a second non-overlapping genomic region and D is the relative chaos. and D is the relative chaos, wherein, if the reference and sample probability distributions do not overlap, JSD equals 1; and if the probability distributions fully overlap, then JSD equals 0; and if probability distributions partially overlap, then JSD is from about 0.1 to about 0.9.
32. The method according to embodiment 31, wherein Q is the allele frequency of DNA from cells that are from about 1 month to about 12 months of age.
33. The method according to embodiment 31, wherein Q is the allele frequency of DNA from cells that are more than about 12 months of age.
34. The method according to any one of embodiments 22 through 33 further comprising steps of: (i) converting genomic DNA to distinguish unmethylated and methylated cytosines
(ii) amplifying the converted DNA to generate the three or more distinct nucleic acid targets; (iii) analyzing data from the sequenced DNA to determine methylation levels at each CpG site; (iv) calculating an unmethylated CpG average for each of the three or more nucleic acid target sequences; (v) calculating epiallele frequencies from (ii) and (iii); (vi) counting the CpGs within the three or more nucleic acid target sequences; (vii) counting a number of methylated CpGs in the three or more nucleic acid target sequences; (viii) calculating chaos of DNA methylation by determining the average percent methylation and the average Jensen-Shannon distance (JSD) at the three or more nucleic acid target sequences.
35. The method according to any one of embodiments 22 through 34, wherein one of the three or more nucleic acid target sequences is amplified using one or a pair of primers chosen from Table 1, 4, or 5.
36. A computer program product encoded on a computer-readable storage medium, wherein the computer program product comprises instructions for: (a) calculating a probability distribution of three or more nucleic acid target sequences in a cell of the subject; (b) calculating a percent methylation and a Jensen-Shannon distance (JSD) at each of the nucleic acid target sequences; (c) determining chaos of DNA methylation by comparing the probability distribution of allele chaos with a control probability distribution to obtain an average percent methylation and an average JSD for each nucleic acid target sequence.
37. The computer program product according to embodiment 36, further comprising a step of correlating the chaos of DNA methylation with the age of the cell.
38. The computer program product according to embodiment 37, further comprising instructions for selecting a treatment for the subject based upon the age of the cell.
39. The computer program product according to any one of embodiments 36 to 38, further comprising instructions for: (d) assigning a score to the amount of chaos of DNA methylation; (c) comparing the score to a first threshold; and (f) classifying the subject as being likely to respond to a treatment, if the score exceeds or falls below a first threshold; wherein each of steps (d), (c), and (f) are performed after step (c), and wherein the first threshold is calculated relative to a first control dataset.
40. The computer program according to embodiment 39, wherein step (d) is performed by using Levene's test of equal variance and corrected by Bonferroni correction.
41. A system comprising the computer program product of any of embodiments 36 through 40 and one or more of: (a) a processor operable to execute a program; and (b) a memory associated with the processor.
42. A system for identifying an age of a cell in a subject, the system comprising:
(a) a processor operable to execute a program; (b) a memory associated with the processor; (c) a database associated with said processor and said memory; and (d) a program stored in the memory and executable by the processor, the program being operable for: (i) calculating a probability distribution of three or more nucleic acid target sequences in a cell of the subject; (ii) calculating a percent methylation and a Jensen-Shannon distance (JSD) at each of the nucleic acid target sequences; (iii) determining chaos of DNA methylation by comparing the probability distribution of allele chaos with a control probability distribution to obtain an average percent methylation and an average JSD for each nucleic acid target sequence.
43. The system according to embodiment 42, wherein the cell is from a sample of the subject.
44. The system according to embodiment 42 or embodiment 43, wherein the cell is a stem cell.
45. A system for identifying chaos of DNA methylation of DNA in a cell in a subject, the system comprising: (a) a processor operable to execute a program; (b) a memory associated with the processor; (c) a database associated with said processor and said memory; and (d) a program stored in the memory and executable by the processor, the program being operable for: (i) calculating a probability distribution of three or more nucleic acid target sequences in a cell of the subject; (ii) calculating a percent methylation and a Jensen-Shannon distance (JSD) at each of the nucleic acid target sequences; (iii) determining chaos of DNA methylation by comparing the probability distribution of allele chaos with a control probability distribution to obtain an average percent methylation and an average JSD for each nucleic acid target sequence.
46. The system according to embodiment 45, wherein the cell is from a sample of the subject.
47. The system according to embodiment 45 or embodiment 46, wherein the cell is a stem cell.
48. A kit comprising one or more primer complementary to at least one target sequence.
49. The kit of embodiment 48, wherein the at least one target sequence comprises three target sequences.
50. The kit of embodiment 48 or 49, wherein the at least one target sequence is chosen from Table 1, 4, or 5.
51. The kit of any one of embodiments 48 through 50, wherein the one or more primer comprises at least one set of amplifying primers, each comprising a forward primer and a reverse primer.
52. The kit of embodiment 51, wherein the at least one set of amplifying primers is chosen from one or more matched set of forward and reverse primers capable of amplifying the at least one target sequence.
53. The kit of embodiment 51, wherein the at least one set of amplifying primers is chosen from one or more matched set of forward and reverse primers chosen form Table 2.
54. The kit of any one of embodiments 51 through 53, wherein the at least one set of amplifying primers comprises at least three sets of amplifying primers.
55. The kit of any one of embodiments 48 through 54, wherein the one or more primer comprises a sequencing primer.
56. The kit of any one of embodiments 48 through 55 further comprising one or more reagent for bisulfite sequencing.
57. The kit of any one of embodiments 48 through 56 further comprising instructions for conducting a method of determining an age or estimated age of a subject.
58. The kit of any one of embodiments 48 through 57 further comprising a computer program product comprising instructions for one or both of (i) sequencing DNA in a cell to obtain at least a portion of nucleic acid sequence of the at least one nucleic acid target sequences; and (ii) analyzing at least a portion of the nucleic acid sequence of the at least one nucleic acid target sequences to determine methylation levels at one or a plurality of CpG sites within the at least one nucleic acid target sequences.
59. The kit of any one of embodiments 48 through 57 further comprising a computer program product comprising instructions for one or both of (i) sequencing DNA in a cell to obtain at least a portion of nucleic acid sequence of three or more of the at least one nucleic acid target sequences; and (ii) analyzing at least a portion of the nucleic acid sequence of the three or more nucleic acid target sequences to determine methylation levels at one or a plurality of CpG sites within the three or more nucleic acid target sequences.
59. A kit comprising: (a) a computer program product comprising instructions for one or both of (i) sequencing DNA in a cell to obtain at least a portion of nucleic acid sequence of at least one nucleic acid target sequences; and (ii) analyzing at least a portion of the nucleic acid sequence of the at least one nucleic acid target sequences to determine methylation levels at one or a plurality of CpG sites within the at least one nucleic acid target sequences; and one or more of: (b) one or more primer complementary to at least one target sequence; and (c) one or more reagent for bisulfite sequencing.
60. The kit of embodiment 59, wherein the at least one target sequence comprises three target sequences.
62. The kit of embodiment 59 or 60, wherein the at least one target sequence is chosen from Table 1, 4, or 5.
63. The kit of any one of embodiments 59 through 62, wherein the one or more primer comprises at least one set of amplifying primers, each comprising a forward primer and a reverse primer.
64. The kit of embodiment 63, wherein the at least one set of amplifying primers is chosen from one or more matched set of forward and reverse primers capable of amplifying the at least one target sequence.
65. The kit of embodiment 63, wherein the at least one set of amplifying primers is chosen from one or more matched set of forward and reverse primers chosen form Table 2.
66. The kit of any one of embodiments 63 through 65, wherein the at least one set of amplifying primers comprises at least three sets of amplifying primers.
67. The kit of any one of embodiments 59 through 66, wherein the one or more primer comprises a sequencing primer.
68. The kit of any one of embodiments 59 through 67 further comprising the one or more primer complementary to at least one target sequence and the one or more reagent for bisulfite sequencing.
69. The kit of any one of embodiments 59 through 68 further comprising instructions for conducting a method of determining an age or estimated age of a subject.
70. A method treating a subject comprising: (a) calculating a probability distribution of three or more nucleic acid target sequences in cells or biological fluids of the subject; (b) calculating a level of DNA methylation probabilistic distribution at each of the nucleic acid target sequences; (c) determining an estimated age of the subject by comparing the probability distribution of allele chaos within the three or more nucleic acid target sequences relative to a control probability distribution to obtain an average percent methylation and an average Jensen-Shannon distance (JSD) for each nucleic acid target sequence; and (d) administering a hypomethylating drug to the subject when the estimate age is greater than the actual age of the subject.
71. The method according to embodiment 70, wherein the hypomethylating drug comprises one or more of 5-azacytidine, 5-aza-2′-deoxycytidine, SGI-110, 5-fluro-2′-deoxycytidine, zebularine, CP-4200, RG108, or nanaomycin.
72. The method according to embodiment 70 or 71, wherein the administering a hypomethylating drug comprises administering a therapeutically effective dose of the hypomethylating drug.
73. The method according to embodiment 72, wherein the therapeutically effective dose is about 0.1 mg/kg to about 2.0 mg/kg.
75. The method according to any one of embodiments 70 through 73, wherein the administering a hypomethylating drug comprises oral, subcutaneous, or intravenous delivery of the hypomethylating drug.
76. The method according to any one of embodiments 70 through 75, wherein the administering occurs over the course of about 1 to about 10 days.
77. The method according to embodiment 1, wherein the step of calculating the average percent methylation of the three or more nucleic acid target sequences comprises: (i) sequencing DNA in the cells or biological fluids to obtain at least a portion of the three or more nucleic acid target sequences; (ii) analyzing the at least a portion of the three or more target sequences to determine methylation levels at one or a plurality of CpG sites within the three or more nucleic acid target sequences.
78. The method according to any one of embodiments 70 through 77, wherein the step of sequencing comprises deep sequencing platform sequencing.
79. The method according to any one of embodiments 70 through 78, wherein the step (a) comprises: amplifying DNA from the cells or biological fluids to generate amplified copies of the three or more nucleic acid target sequences, wherein the three or more nucleic acid target sequences comprise a plurality of CpG sites.
80. The method according to embodiment 79, wherein step (a) and/or (b) comprises: (i) analyzing the amplified copies of the three or more nucleic acid target sequences to determine an individual value of methylation levels at each CpG site at the three or more nucleic acid target sequences; and (ii) calculating an unmethylated CpG average for each of the three or more nucleic acid target sequences.
81. The method according to any one of embodiments 70 through 80, further comprising calculating epiallele frequencies.
82. The method according to embodiment 81, wherein the step of calculating epiallele frequency is calculated by: (i) determining a level of methylation levels at CpG site across DNA in the cells or biological fluid; and (ii) calculating an unmethylated CpG average for each of the three or more nucleic acid target sequences.
83. The method according to embodiment 82, wherein the step of calculating epiallele frequency is further calculated after steps (i) and (ii) of embodiment 7, by performing steps: (iii) identifying CpGs only within the three or more nucleic acid target sequences; and (iv) counting the number of methylated CpGs within the three or more nucleic acid target sequences.
84. The method according to any one of embodiments 70 through 83, wherein the average JSD is calculated by the formulas:

$$\frac{1}{n} {\sum }_{i = 1}^{n} {( JSD )}_{i}$$

$$M = \frac{( P + Q )}{2}$$

$$JSD ( P ‖ Q ) = \sqrt{\frac{{D}_{KL} ( P ‖ M ) + {D}_{KL} ( Q ‖ M )}{2}}$$

wherein JSD is the Jensen-Shannon Distance for a nucleic acid target sequence and n is the number of epialleles.
85. The method according to any one of embodiments 70 through 84, further comprising calculating methylation chaos.
86. The method according to embodiment 85, wherein the Jensen-Shannon distance of methylation is calculated by:

$$\frac{1}{n} {\sum }_{i = 1}^{n} {( JSD )}_{i}$$

$$d = \sqrt{\frac{D ( {P}_{L}^{( 1 )} , Q ) + D ( {P}_{L}^{( 2 )} , Q )}{2}}$$

$$D ( P , Q ) = {\sum }_{ℓ} P ( ℓ ) {log}_{2} ( \frac{P ( ℓ )}{Q ( ℓ )} )$$

wherein, [symbol] is the frequency of methylated CpGs on an epiallele, Q is the control probability distribution of a control allele; P is the probability distribution of allele chaos; P_{L}⁽¹⁾ is the probability mass function (PMF) of methylation within a first genomic region and P_{L}⁽²⁾ is the PMF of methylation within a second non-overlapping genomic region and D is the relative entropy, wherein, if the reference and sample probability distributions do not overlap, JSD equals 1; and if the probability distributions fully overlap, then JSD equals 0; and if probability distributions partially overlap, then JSD is from about 0.1 to about 0.9.]
87. The method according to embodiment 86, wherein Q is the allele frequency of DNA from cells that are from about 1 month to about 12 months of age.
88. The method according to embodiment 86, wherein P is the probability distribution of allele frequency in DNA from a cell that is more than about 12 months of age.
89. The method according to embodiment 70, further comprising: (i) amplifying DNA from the cells or biological fluids to generate the three or more nucleic acid target sequences to produce amplified DNA and sequencing the amplified DNA to produce sequence data; (ii) analyzing the sequence data to determine methylation levels at each CpG site; (iii) calculating an unmethylated CpG average for each of the three or more nucleic acid target sequences; (iv) calculating epiallele frequencies from (ii) and (iii); (v) counting the CpGs within the three or more nucleic acid target sequences; (vi) counting a number of methylated CpGs in the three or more nucleic acid target sequences; (vii) calculating methylation chaos by determining the average percent methylation and the average Jensen-Shannon distance (JSD) at the three or more nucleic acid target sequences.
90. The method according to any one of embodiments 70 through 89, wherein the amplifying DNA comprises amplifiying at least one of the three or more nucleic acid target sequences with primers comprising one or a pair of primers comprising at least about 75% sequence identity to a sequence in Table 2, optionally one or a pair of primers comprising a sequence in Table 2.
91. The method according to embodiment 89 or embodiment 90, wherein at least a portion of the DNA is treated with sodium bisulfite prior to being amplified.
92. The method according to embodiment 91, wherein the sulfite treated DNA is amplified by the Polymerase Chain Reaction, and optionally wherein the analyzing comprises comparison of the sequence data to non-bisulfite sequence information, further optionally wherein the non-bisulfite sequence information is obtained from one or both of archived genome sequence information or sequencing of amplified, untreated DNA from the cells or biological fluids.
93. The method according to any one of embodiments 70 through 92, wherein the cells are cancer cells.
94. The method according to any one of embodiments 70 through 92, wherein the cells are stem cells.
95. The method according to embodiment 94, wherein the stem cells are adult stem cells.
96. The method according to any one of embodiments 70 through 95, wherein the method is free of a step of correlating the amount of differentiation of the cell to the age of the cell.

The following non-limiting examples include further embodiments herein. Still further embodiments herein include supplementing or substituting one or more detail in an embodiment with one or more detail from the following examples.

### EXAMPLES

### Example 1

DNA was extracted from whole blood obtained from 155 healthy individuals and deposited into the biobank of the National Institute for Neurologic Diseases (NINDS). The median age of these individuals was 51 (range 19 to 91). Other clinical characteristics are described in Table 3. To establish baseline allelic distribution of methylation, two healthy cord blood DNA samples obtained from the Department of Stem Cell Transplantation and Cellular Therapy, The University of Texas MD Anderson Cancer Center, Houston TX, were studied. As a validation data set, whole blood DNA obtained from 300 patients referred to the Cooper University Hospital for management of trauma was used. The median age of these individuals was 62 (range 18 to 101). The clinical characteristics of these patients are described in Table 3.

TABLE 3
Characteristics of subjects in the testing
and validation sample cohorts.

| | | Age range | | | |
|---|---|---|---|---|---|
| Cohort | Race | (median) | | Male | Female |
| NINDS | White | 20-91 | (50) | 51 | 50 |
| | Black/African | 19-91 | (60) | 15 | 15 |
| | American | | | | |
| | American Indian/ | 32-69 | (49.5) | 3 | 5 |
| | Alaska Native | | | | |
| | Asian | 22-80 | (48) | 9 | 7 |
| | All | 19-91 | (51) | 78 | 77 |
| CUH | White | 18-101 | (65) | 138 | 90 |
| | Black/African | 18-86 | (36) | 46 | 14 |
| | American | | | | |
| | Asian | 78-79 | (79) | 1 | 1 |
| | Other | 28-94 | (56) | 9 | 1 |
| | All | 18-101 | (62) | 194 | 106 |
| Leukemia | CML | 16-76 | (50) | 19 | 10 |
| | AML | 48-81 | (67) | 6 | 5 |

### Reduced Representation Bisulfite Sequencing

Blood DNA samples from 19 NINDS biobank individuals aged 22-80 years were analyzed for DNA methylation using Reduced Representation Bisulfite Sequencing (RRBS, Gu H, Smith Z D, Bock C, Boyle P, Gnirke A, Meissner A. Preparation of reduced representation bisulfite sequencing libraries for genome-scale DNA methylation profiling. Nat Protoc. 2011 April; 6 (4): 468-81. doi: 10.1038/nprot.2010.190. Epub 2011 Mar. 18. PMID: 21412275.) using the New England Biolabs (NEB) protocol for methylated adaptors as described previously (Zhang H, Pandey S, Travers M, Sun H, Morton G, Madzo J, Chung W, Khowsathit J, Perez-Leal O, Barrero C A, Merali C, Okamoto Y, Sato T, Pan J, Garriga J, Bhanu N V, Simithy J, Patel B, Huang J, Raynal N J, Garcia B A, Jacobson M A, Kadoch C, Merali S, Zhang Y, Childers W, Abou-Gharbia M, Karanicolas J, Baylin S B, Zahnow C A, Jelinek J, Graña X, Issa JJ. Targeting CDK9 Reactivates Epigenetically Silenced Genes in Cancer. Cell. 2018 Nov. 15; 175 (5): 1244-1258.e26. doi: 10.1016/j.cell.2018.09.051. Epub 2018 Oct. 25. PMID: 30454645; PMCID: PMC6247954.). Briefly, 1 microgram of genomic DNA was spiked with 100 picograms of lambda phage DNA as the unmethylated standard and digested with MspI endonuclease at C′CGG sites. The ends of restriction fragments were filled in, 3′-dA tailed and methylated adaptors (NEB E7535) were ligated to the ends of restriction fragments. Bisulfite treatment using the Epitect kit (Qiagen) follows. Bisulfite-converted libraries were amplified using EpiMark Taq DNA polymerase (NEB) and primers with dual barcode indices (NEB E6440). The libraries were pooled and sequenced at Novogene (Sacramento, CA) on Illumina HiSeqX instrument using paired end reads of 150 bases. Bismark v0.23.1 (Krueger F, Andrews SR. Bismark: a flexible aligner and methylation caller for Bisulfite-Seq applications. Bioinformatics. 2011 Jun. 1; 27 (11): 1571-2. doi: 10.1093/bioinformatics/btr167. Epub 2011 Apr. 14. PMID: 21493656; PMCID: PMC3102221.) was used to align the sequences to hg19 human genome assembly; and methylKit v1.22.0 (Akalin A, Kormaksson M, Li S, Garrett-Bakelman F E, Figueroa ME, Melnick A, Mason C E. methylKit: a comprehensive R package for the analysis of genome-wide DNA methylation profiles. Genome Biol. 2012 Oct. 3; 13 (10): R87. doi: 10.1186/gb-2012-13-10-r87. PMID: 23034086; PMCID: PMC3491415.) was used to analyze differential methylation.

### Selection of Aging Target Loci

Candidate biomarkers of age-related DNA methylation using RRBS data from 19 NINDS biobank individuals aged 22-80 years and 33 umbilical cord blood samples publicly available at GEO GSE109538 were identified as follows. Using linear models of methylation changes with age, we selected differentially methylated regions of interest based on four criteria: (i) At least 2% change per 10 years. (ii) Average DNA methylation in the samples from young donors (22-25 yo) less than 25% (which enriches for hypermethylation with age) or more than 75% (which enriches for hypomethylation with age). (iii) Concordant methylation changes in four or more CpG sites for hypermethylation with age or two of more hypomethylated CpG sites in a 500 bp window. (iv) Increase of standard deviation of DNA methylation from the young to the middle age to the old group. 85 genomic regions meeting the above criteria (Table 4) were identified. From these, 12 targets for multiplex bisulfite PCR were selected. A complementary approach was also used to identify loci that undergo hypermethylation with aging. RRBS sequencing reads from five donors aged 22-25 years were merged into a “young” pool and reads from five donors aged 77-80 years into an “old” pool. Using methylKit, methylation differences were calculated between “old” and “young” pool at 3,069,051 CpG sites covered with 50 or more reads. 1179 differentially methylated CpG sites were selected with FDR<0.05, methylation difference between “old” and “young” greater than 25% and methylation in the “young” pool less than 10%. Next, regions with at least four neighboring CpG sites within a 100-base distance were selected. Calculated were Pearson correlation and linear regression of DNA methylation with age using RRBS data from 17 individual NINDS samples and in 7 mid age samples (35-61 yo donors) not used in the original selection as a validation step. Based on these criteria, 11 regions (Table 5) with the highest correlation of methylation increase with age (Pearson r 0.36-0.54 in the validation set of mid age samples) were selected for additional screening. Of these. 8 targets were selected for multiplex bisulfite PCR.

TABLE 4
Eighty-five candidate aging targets.

| Candidate | | | | | SEQ ID |
|---|---|---|---|---|---|
| Locus | Chromosome | Start | End | Sequence | NO. |
| set1.01 | 1 | 1,176,166 | 1,176,221 | CGCGGCCCTGGGTCCCATTTC | 61 |
| | | | | TGGCATGTCCATCTGTCATCA | |
| | | | | CAGCTCCTACCTCCG | |
| set1.02 | 1 | 12,100,075 | 12,100,254 | CGGGGAAGTCTGAGACTGCA | 62 |
| | | | | GTGCGTGGTGATCACAACAC | |
| | | | | TGCACTCCAGCCTGAGCAAC | |
| | | | | AGAGTGAGACCATGTCTAAA | |
| | | | | AATAAATAAATAAATAAAAA | |
| | | | | TGCCGGGCGTGGTGGCTCAA | |
| | | | | GCCTGTAATCCCAGCACTTTG | |
| | | | | GGAGGCCAAGGTGGGCAGAT | |
| | | | | CACTTGAGGTCAGGAGTTC | |
| set1.03 | 1 | 19,110,747 | 19,110,823 | CGGGCCAGATGCGCCTGAGC | 63 |
| | | | | GCGGCCTCGTTATGTATTCAT | |
| | | | | GAGCTGTGAGGAAAAGAAAT | |
| | | | | AAAAGGATTCATTATC | |
| set1.04 | 1 | 24,718,313 | 24,718,431 | CGGATTAAAAAAAAAATTCC | 64 |
| | | | | CCACTTTCTTTCCCTCTCGGC | |
| | | | | AATTTATCGGACTTCCCCCCT | |
| | | | | CCAGCTCTTAAATTAGTGAGA | |
| | | | | TGTGGTCACATAAAGTACCTT | |
| | | | | AAACAGGCTGTCCCG | |
| set1.05 | 2 | 47,571,648 | 47,571,711 | CGCGATCTCGGCCCACTGCA | 65 |
| | | | | ACCTCCGCCTCCCAGGTTCAG | |
| | | | | GCGATTCTCCAGCCTCAGCCT | |
| | | | | CCG | |
| set1.06 | 2 | 49,351,370 | 49,351,430 | CGTGATCCACCCACCTCGGCC | 66 |
| | | | | TCTCAAAGTGCTGGGATTACA | |
| | | | | GCCATGAGCCACCACACCCG | |
| set1.07 | 2 | 52,857,857 | 52,857,917 | CGTGATCTGCCTGCCTTGGCC | 67 |
| | | | | TCCCAAAGTGCTGGGATTAC | |
| | | | | AGGTGTGAGCCACTGGGCCC | |
| | | | | G | |
| set1.08 | 2 | 96,192,255 | 96,192,283 | CGCCGCTCCCCGAGAGGCCG | 68 |
| | | | | CAAAAGCCC | |
| set1.09 | 2 | 207,563,052 | 207,563,209 | CGGATCTCCAGTTTTTCGGTG | 69 |
| | | | | TACCAAGCAGACCTATTTTAC | |
| | | | | CTCCATGGGGAGCAATTTCA | |
| | | | | GTTCTGGGTTAGCTAGGGTCA | |
| | | | | GGAAGCATGGGAAGGGAAGG | |
| | | | | GGAACAAAGTGAGCAGGAGC | |
| | | | | TGAGTCCTGAGGCCTCTTGTC | |
| | | | | CCCTTC | |
| set1.10 | 2 | 236,785,127 | 236,785,134 | CGTCTCCCG | 70 |
| set1.11 | 3 | 47,051,298 | 47,051,367 | CGTGACCCCCTCTGGGCCCAC | 71 |
| | | | | TCGCCCCCCCTCCGCGTGGCC | |
| | | | | GTCGCTGAACTGGGGCCTCA | |
| | | | | GTTTACCCG | |
| set1.12 | 3 | 51,741,211 | 51,741,521 | CGCTGCCGCTGCCGACCTTTT | 72 |
| | | | | TGGCCCTTACCTCACGTCCCA | |
| | | | | GGGTCCTGCGGGCCCTCAAG | |
| | | | | TTGTGGGGCGCCCGCGCTGCT | |
| | | | | GTGTCCAGACAGCGTTCCCTG | |
| | | | | AGAGCTCCGGGAAGCGGGAA | |
| | | | | GACAGCCCCGGGCGTCCCGC | |
| | | | | CTTTCTTCTCCAGAAAACGCA | |
| | | | | CGCCCCACATCGCACTCCCCC | |
| | | | | GTTCCTCCTGCTCCAGCGTCC | |
| | | | | TCTGGTCCTTCTTTCTGTCTGT | |
| | | | | GCCTCCGTCTTTGTCTCAACC | |
| | | | | TCTCAGGCTTGCTCGCTCCCT | |
| | | | | GCCCAGATTTTGTGGCCCAGG | |
| | | | | CTCCTGGCTGTCTGACTCCG | |
| set1.13 | 3 | 65,342,466 | 65,342,540 | CGCTTCTCTGGGGACCGCCTC | 73 |
| | | | | TTGGGGCCGTTGGCGGCTGCC | |
| | | | | GCGCGCTCGGCCTGCGCGTCC | |
| | | | | CTCCGTCCCTCCG | |
| set1.14 | 3 | 71,478,153 | 71,478,206 | CGACATGCAGACAGTAGTCG | 74 |
| | | | | GCTGACATTTTTTGCTATTTC | |
| | | | | CGTTATTACAGCCG | |
| set1.15 | 4 | 3,898,076 | 3,898,105 | CGCAGCTGACAAACAGGGCG | 75 |
| | | | | GCTTGTCGCCG | |
| set1.16 | 4 | 147,558,436 | 147,558,491 | CGCCAGCGCCTCTCAGGCCTG | 76 |
| | | | | CCGCCTGCTCTCGCACCTGCT | |
| | | | | CGCCTTCCCCAGGCG | |
| set1.17 | 5 | 926,945 | 927,022 | CGCGCCCTCGGCTTCCCTGTT | 77 |
| | | | | GCTCAGGGTTATTTCTTCCTG | |
| | | | | CCATCAGCTGGAGAAGCGCT | |
| | | | | CTCCGAATATTTCCCCG | |
| set1.18 | 5 | 1,137,330 | 1,137,379 | CGGGGCCACCTGTGCCCTCTT | 78 |
| | | | | CCCAGAGCACTCGAGGCCAG | |
| | | | | GCACGATCCG | |
| set1.19 | 5 | 2,334,866 | 2,334,886 | CGAGAATAAGGGCTCGGCTC | 79 |
| | | | | CG | |
| set1.20 | 5 | 3,599,703 | 3,599,776 | CGGAGAAGGCCGAGGACGAC | 80 |
| | | | | GAGGAGATCGACCTGGAAAG | |
| | | | | CATCGACATTGACAAGATCG | |
| | | | | ACGAGCACGATGGC | |
| set1.21 | 5 | 42,952,584 | 42,952,647 | CGGAGGCTGAGGCAGAAGAA | 81 |
| | | | | TCGATTGAACTCAGGAGGCA | |
| | | | | GAGGTTGCAGTGAGCCAAGA | |
| | | | | TCGC | |
| set1.22 | 5 | 43,000,495 | 43,000,549 | CGAGCTCTCCTGGGCCGACCT | 82 |
| | | | | AGATTTCCACTGCCACATACT | |
| | | | | TTCCGCTCCCTGCG | |
| set1.23 | 5 | 134,363,517 | 134,363,717 | CGCTGTAAACAGGGGCGCGG | 83 |
| | | | | GCCGGAGAGCGGGTGTGCAA | |
| | | | | AGTGGGCGCAGGGCCCTGGG | |
| | | | | GCCGCGCCCCTTGCTCTGCCG | |
| | | | | GCTCGACTCTTGCACGGCGG | |
| | | | | GCGGTGAGGAGGGGGCTGTT | |
| | | | | CGCCCAGACAGAGGGCCACC | |
| | | | | TCCTAGCCCGGGAGCAGAGC | |
| | | | | AGAGGGCCTGGGCCTGCAGC | |
| | | | | TAAGCTCAAGGCTGGGGTGT | |
| | | | | T | |
| set1.24 | 5 | 140,782,793 | 140,782,870 | CGGGAGGAGCTCTGTGCTCA | 84 |
| | | | | GAGCCCGCGGTGTCTGGTGA | |
| | | | | ACTTTAAAGTCCTGGTTGAAG | |
| | | | | ACAGAGTGAAACTGTAC | |
| set1.25 | 5 | 169,137,720 | 169,137,797 | CGGTTCTCCACTTATTACACA | 85 |
| | | | | TATTATTACTTTGCTCAGTGT | |
| | | | | GTCTCCCCATACCCAATGCCT | |
| | | | | TCGAATTGATGACCCG | |
| set1.26 | 5 | 174,673,988 | 174,674,022 | CGGTATTTCCCTGAAAATAAA | 86 |
| | | | | TAATCCAGGCATCCG | |
| set1.27 | 6 | 10,416,497 | 10,416,523 | CGCGGGCGTGCTCGCGAGGC | 87 |
| | | | | AGAGCCCG | |
| set1.28 | 6 | 32,116,983 | 32,117,049 | CGGACCAGGGGCGTTTTTAG | 88 |
| | | | | GGATCCCAGTAGTTCTCGTGG | |
| | | | | TGCTGCGCGGCGATGATGAT | |
| | | | | GACTAC | |
| set1.29 | 6 | 150,040,098 | 150,040,162 | CGGACGGGCGCGGTGTCTCA | 89 |
| | | | | CGCCTGTAATCCCAGCACTTT | |
| | | | | GGGAGGCCGAGGCGGGCGGA | |
| | | | | TCAC | |
| set1.30 | 6 | 159,360,150 | 159,360,216 | CGCCTTCTCTGGAAGGCTCCC | 90 |
| | | | | TCATCCTCTGTCGTAGTCCAG | |
| | | | | GGCTCCCTCCTAGACCTGCGG | |
| | | | | CCCCG | |
| set1.31 | 7 | 2,902,655 | 2,902,876 | CGCCTTGGCAGTGCTCGCTAA | 91 |
| | | | | GTGTTTGCATTTTTTTCCCTCC | |
| | | | | CTGTAACCGCTAGACCACCA | |
| | | | | CGGAACTTGCATTTTTTGCTA | |
| | | | | CTGGATGACAGGTCTTCCTCC | |
| | | | | TCTCCCAGGGTGGCTGTCTGG | |
| | | | | CAGGTTTCCCCACTTCCTGCA | |
| | | | | GTCTTCTCTGCCCTAGGGGAC | |
| | | | | CAGTAGCCATGTTTCTGCCCC | |
| | | | | AACAAGTAACCTCCTTGCCCT | |
| | | | | GTCCTGGCTCCCG | |
| set1.32 | 7 | 8,482,233 | 8,482,300 | CGACGTAGGCTTCATACCCTC | 92 |
| | | | | CCTTCGGAAACTCAGTCCGCT | |
| | | | | GACCAAAGCCGCAGTGTTCA | |
| | | | | GGCCCCG | |
| set1.33 | 7 | 15,725,521 | 15,725,591 | CGCTTTTCCTCTTGCCGCCGC | 93 |
| | | | | TTCGCTTCTCCGCCTCCGCAG | |
| | | | | GTGACAGTGCCTGGCGGCCG | |
| | | | | TAGTCCCCCG | |
| set1.34 | 7 | 53,390,023 | 53,390,141 | CGGTGGGTTCTTGGTCTTGCT | 94 |
| | | | | GACTTCCAGAATGAAGCCAC | |
| | | | | AGACCCTTGCAGTGAGTGTTA | |
| | | | | TAGCTCTTAAAGACGGTATGT | |
| | | | | CCTAAGTTTATTCCTTCAGAT | |
| | | | | GTTCAGGTGTGTCCG | |
| set1.35 | 7 | 100,231,611 | 100,231,673 | CGCGAGCCGCAGTGCAGGGC | 95 |
| | | | | CCTCCGCGGACCCATTTTCTC | |
| | | | | CCATCACCACCAGGGCGGCG | |
| | | | | CCG | |
| set1.36 | 7 | 101,936,505 | 101,936,520 | CGCTATTTTTACCGCCG | 96 |
| set1.37 | 7 | 137,831,938 | 137,832,065 | CGGGAGGAACAAACAACTCC | 97 |
| | | | | AGATGCGCCGCCTTAAGAGG | |
| | | | | TGTAACACTCACCACGAAGG | |
| | | | | TCGGCAGCTTCACTCCTGAGC | |
| | | | | CAGCGAAACCACGAACCCAC | |
| | | | | CAGAAGGAATAAACTCCGAA | |
| | | | | CACATCC | |
| set1.38 | 8 | 102,504,808 | 102,504,859 | CGGCTAGGTGAGGGCGCGAG | 98 |
| | | | | CGGGCGAGCGAGCGAGAGTG | |
| | | | | GTGAGGGGGGAC | |
| set1.39 | 9 | 1,046,248 | 1,046,284 | CGGGGCAGGATCCGCTCCCG | 99 |
| | | | | CGATTAGCTCTGGGAGC | |
| set1.40 | 9 | 124,988,309 | 124,988,349 | CGGGCCAGAAATGGGGACCT | 100 |
| | | | | CAGAGCTCCACCGAGGCGCT | |
| | | | | C | |
| set1.41 | 9 | 132,920,862 | 132,920,895 | CGGACATGGTGGCTCACGCC | 101 |
| | | | | TGTAATCCCAACAC | |
| set1.42 | 9 | 136,075,470 | 136,075,549 | CGGGGACGGGCAAAGGAACG | 102 |
| | | | | CAGCTCGTGAGTGGCCCAGA | |
| | | | | GAGCGGGAACCAGAGCGCCC | |
| | | | | CGACGGCAGCGGAAGCCACC | |
| set1.43 | 10 | 22,766,278 | 22,766,349 | CGCACAACTAACGCAATAGC | 103 |
| | | | | CTGAGGGGTTTGGTAAACAG | |
| | | | | AAGCGGCCCCAGGAGGGGGT | |
| | | | | GGGATTCGCCCCG | |
| set1.44 | 10 | 116,003,431 | 116,003,487 | CGATGGTAGAGAAGGCAGAC | 104 |
| | | | | ATTATCCTGCAAAACTGCCTT | |
| | | | | CAGCGATCCCCAATCCG | |
| set1.45 | 10 | 116,636,807 | 116,636,857 | CGTCTGAAGTTTTGTTCTGTT | 105 |
| | | | | TATGCCTCCAAGCGTGTTGCC | |
| | | | | GACACATCCG | |
| set1.46 | 10 | 135,278,976 | 135,279,052 | CGGGGCAGCGAGGCGTCCCT | 106 |
| | | | | GTGGGGCTGCCCTGCGGAGC | |
| | | | | GGTGGGGACGCGGAGACCGC | |
| | | | | GCGCACGAGGAGGACGC | |
| set1.47 | 11 | 32,448,225 | 32,448,293 | CGGGGGAAAAAAAGGAAAA | 107 |
| | | | | AAAAAGGTTTCTCCAGTCCGC | |
| | | | | GCGCCTCAGGCGTTAGAAAT | |
| | | | | AGAAGGGGC | |
| set1.48 | 11 | 65,790,325 | 65,790,383 | CGGATCTCGACTTCAGCGAA | 108 |
| | | | | GGTCTGGGCCACCAGCATGA | |
| | | | | CGGTGTTGAGGCAGACAAC | |
| set1.49 | 12 | 52,213,983 | 52,214,040 | CGGGCGGAGATGGCGAGCTT | 109 |
| | | | | CCAGTCCACAATAAATAGGA | |
| | | | | AAAACCTGAGTACACGGC | |
| set1.50 | 12 | 54,367,479 | 54,367,542 | CGACCGTTTCTTCGACAACGC | 110 |
| | | | | CTACTGCGGTGGCGGCGACC | |
| | | | | CGCCCGCCGAGCCCCCCTGCT | |
| | | | | CCG | |
| set1.51 | 12 | 57,857,472 | 57,857,486 | CGCCATGTTCAACTCG | 111 |
| set1.52 | 12 | 125,077,775 | 125,077,853 | GCCTTTCCCTCTCCTTGTTTCC | 112 |
| | | | | GAAGGAGAGCCTGCCTCTCG | |
| | | | | CCCCGAGGTTGAACATTCCA | |
| | | | | GCTCCTTGCTCTCCCCG | |
| set1.53 | 12 | 132,671,062 | 132,671,184 | CGGACACCCCCAGGAAGGCC | 113 |
| | | | | ACGTTCTGAGGTTAGAAAGG | |
| | | | | GAAAAAATCAGATCTCACTG | |
| | | | | AACTATGCCCGTTAAGGGGG | |
| | | | | AAATGATCCCAGTTTTGAAAT | |
| | | | | CCATCTTCAAAGCCCTGTAAG | |
| | | | | C | |
| set1.54 | 13 | 41,054,583 | 41,054,611 | CGGAGAGCAGCCAAGGGCAC | 114 |
| | | | | CCCCTTGCC | |
| set1.55 | 13 | 49,795,464 | 49,795,525 | GCCCAGCTCTGGGCGCCGCTT | 115 |
| | | | | CCAGCTCCCTTTCCTATTTCG | |
| | | | | ATTCCAGCCTCCACCCGCCG | |
| set1.56 | 13 | 111,465,066 | 111,465,130 | GGGCAGGGTCCCTCCTGCAG | 116 |
| | | | | AAACCGCTCCTGCCCGCAGC | |
| | | | | GCGCGCGCTTGCTGCCTCCCG | |
| | | | | CCCG | |
| set1.57 | 14 | 76,939,949 | 76,939,988 | CGCTGCGGCTGACCCTGCAC | 117 |
| | | | | ACTCGGCTATTTTTACTTCC | |
| set1.58 | 15 | 31,320,641 | 31,320,880 | GCTTGCCCTCCTCATCATATA | 118 |
| | | | | GGTTCTCACCACAAGGAGCT | |
| | | | | GAAAGAAAAAAATAGTTTTG | |
| | | | | TCTTTGCTTTTTACATATAAT | |
| | | | | GAAAAGGATAAATATCTCTT | |
| | | | | ACATATGTTTTTCAATACCTA | |
| | | | | TTTCTTTTTTAATATGATTCTT | |
| | | | | TCTTATTTCATAAGCAATATT | |
| | | | | TTTTCCATGGGAAAAAATAA | |
| | | | | ACTCTGTAGCCCCAGCTACTC | |
| | | | | GGGAGTCTGAGGCAGGAGAA | |
| | | | | TGGCGTGAACCCG | |
| set1.59 | 15 | 31,775,406 | 31,775,484 | CGGTGGACCAGGGCATGTAA | 119 |
| | | | | AAAAGACACCGACACAATGG | |
| | | | | AAAAGAAATCCTCGAAGGTA | |
| | | | | GAACCTCGCCGCCCGCGCC | |
| set1.60 | 15 | 66,965,879 | 66,965,952 | CGGCCCATGCTCTTGCAAGG | 120 |
| | | | | GCACTGCGGTTTTTGCTTGGG | |
| | | | | AAACGGGTAGCAAACAGCTA | |
| | | | | AGACTCCCAGAAC | |
| set1.61 | 16 | 8,767,923 | 8,767,984 | CGCTGGGGCCAGGCGGAGGA | 121 |
| | | | | AAGTAGCTGGGAGCAAGAAG | |
| | | | | GGCTGGCAGGGCCCTGAGCG | |
| | | | | CC | |
| set1.62 | 16 | 73,517,085 | 73,517,225 | CGGAGGAAGAGAACGCGTGG | 122 |
| | | | | GCCCCTGCTCCAAGTGCCAGC | |
| | | | | GCACGCCTGGCCCAGAGGTC | |
| | | | | CATCGGGCTGCCAGGACAAA | |
| | | | | TGCGTACGCATAGACACGTG | |
| | | | | CACGGAGCCTCCGAGAGAGA | |
| | | | | GCGAGAGACCAAGAGCGAGC | |
| set1.63 | 16 | 84,541,109 | 84,541,229 | CGGAAGCCACGGCTGACTTG | 123 |
| | | | | TGCAGTAGAGGAAGTCGAGT | |
| | | | | TCATTTTATTGAATTTATTCTC | |
| | | | | ATTTTCAGTTTGAATGGCCAC | |
| | | | | ATGTGGCAGGCAGTTATTCTA | |
| | | | | TTGTGCAGTGCTTGCCG | |
| set1.64 | 17 | 8,218,888 | 8,218,961 | GAGCGGTCAGTGGCTTTCCGT | 124 |
| | | | | CCTTCCAGGGAACCTGCCCTT | |
| | | | | AGGCTGCTGGGCACGCCCTTT | |
| | | | | CCTCTCTCCCG | |
| set1.65 | 17 | 37,720,009 | 37,720,167 | CGCCGAGGAGGAAGGGAGAG | 125 |
| | | | | GGAGAGTGAGAGTGAGGAAG | |
| | | | | GGGGGAGAGAAGGGGGAAA | |
| | | | | AACCAGCAGCTGTCGGCCTA | |
| | | | | ATTCTTCTAACACTCTGCTTG | |
| | | | | TGGTCATATTAGAAAAACAG | |
| | | | | ATTATGCCCCTCGGTGCCACT | |
| | | | | CACTTATACTTGACATAC | |
| set1.66 | 17 | 40,700,536 | 40,700,595 | GCTTCCTGCACCAGCTGCTGA | 126 |
| | | | | TGCGCCTGGACGACCCCTTTG | |
| | | | | GCTTCGACTACGCCGCCG | |
| set1.67 | 17 | 41,321,282 | 41,321,347 | GCCTCCTGGGTTCAAGCGATT | 127 |
| | | | | CTCCTGCCTCAGCTCCTGAGT | |
| | | | | AGCTGGCGCGCGCCACCACG | |
| | | | | CCCG | |
| set1.68 | 17 | 78,912,389 | 78,912,461 | GTTGCGGGATTATTTCTAAAT | 128 |
| | | | | CAGAAAATGTGCGGAGGGAG | |
| | | | | CCATTTGACACCTTTTGTGGT | |
| | | | | TACTGTTTCCG | |
| set1.69 | 18 | 37,379,537 | 37,379,874 | CGGCTAACACGGTGAAACCC | 129 |
| | | | | CGTCTCTACTAAAAATACAG | |
| | | | | AAAAAAAATTAGCCAGCCGT | |
| | | | | GGTGGCGAGTGCCTGTAGTC | |
| | | | | CCAGCTACTTGGGAGGCTGA | |
| | | | | GGCAGGAGAATGGCGTGAAC | |
| | | | | CCGGGAGGCGGAGCTTGCTG | |
| | | | | TGAGCCAAGATCGCGCCACT | |
| | | | | GCACTCCAGCCTGGGCGACG | |
| | | | | GAACGAGACTCAGTCTCAAA | |
| | | | | AAAAAAAAAAAAAAAATCCT | |
| | | | | GGGGAAGAACCTCTTATTCTT | |
| | | | | ATGCAAGTGGTTTCTCCACCA | |
| | | | | GGGAGAAAAGTTTGATTACT | |
| | | | | GGCTGATGGAGCTGAATCTCT | |
| | | | | TGGCGGGGAAGGGGAAGGCT | |
| | | | | CCAGCCGTTCATGGC | |
| set1.70 | 18 | 72,916,865 | 72,917,000 | GGCTTGACCGTGACCTTGGCC | 130 |
| | | | | TCGCAGGCACCCCCATTTCTC | |
| | | | | ACCCCCGCTCTCCCGCCCCGC | |
| | | | | CGTCTTCTAAATTGTCTGCGT | |
| | | | | CGTCGGTGAAGGAGGCTTAG | |
| | | | | GCTGGCTGACGGCAGGAGCC | |
| | | | | CGCGGCGGCTCG | |
| set1.71 | 18 | 77,376,961 | 77,377,026 | CGGGAAAGGGAGAGGACGCC | 131 |
| | | | | CCAGGAATGACGGCGCTGAG | |
| | | | | CCCCTGCGGCGGGACAGGCT | |
| | | | | CTGAGC | |
| set1.72 | 19 | 518,746 | 518,820 | CGGGGATGGGGGGTAGGAGG | 132 |
| | | | | AGAGGGGAGGCCAGGGCTGG | |
| | | | | CTGGGGGGTCGGGGAGGCTA | |
| | | | | GGGCATAGGCCTGGC | |
| set1.73 | 19 | 20,606,301 | 20,606,367 | GATGGAGTCTCGCTCTGTCGC | 133 |
| | | | | GCATATTGGAATGCGACGGC | |
| | | | | GCGATCTCGGCTCACGGCAA | |
| | | | | CCTCCG | |
| set1.74 | 19 | 35,800,586 | 35,800,611 | CGGCGGGGTCGTAGTGAGGT | 134 |
| | | | | CAAGGC | |
| set1.75 | 19 | 36,247,217 | 36,247,270 | GAGTCACGGGACCTCGGCAG | 135 |
| | | | | CTACTGGTAGCCTTCCCCCAC | |
| | | | | TTCAGAGTGGCCG | |
| set1.76 | 19 | 41,120,959 | 41,121,031 | GCCCACAGACCCGCCCCTTGC | 136 |
| | | | | CTTTTCTTACTTTCCAGGCCTT | |
| | | | | CCCTCCCGCCCCGCTCTTTCA | |
| | | | | CCCCTCCCG | |
| set1.77 | 19 | 52,996,353 | 52,996,441 | GTAATTTTCCTGCTCAAAACC | 137 |
| | | | | TTTTTCTGACTCTCCCGCCCC | |
| | | | | GTGCTTCTTAAAGTCCTCACC | |
| | | | | CGCGAGGTGGATTCCCGCCCT | |
| | | | | GGGCG | |
| set1.78 | 19 | 55,964,378 | 55,964,441 | GAGTCCGTGTCCCACAGTCTG | 138 |
| | | | | AGACTCTTCTTCCCCTCCCCT | |
| | | | | TCCCGCCCCGTGAAGTGGCCC | |
| | | | | G | |
| set1.79 | 20 | 260,147 | 260,205 | CGGGACTCTCATCCGTTCGGA | 139 |
| | | | | AACGCACGTGTACCCATCATC | |
| | | | | TCACATCCCTGAGGTGC | |
| set1.80 | 20 | 21,492,282 | 21,492,312 | CGAAGCTGCGCAAACATTCT | 140 |
| | | | | GTAAACACGGC | |
| set1.81 | 21 | 47,716,529 | 47,716,559 | CGGTCCACATGGTTAACACG | 141 |
| | | | | CACGCAAGCCC | |
| set1.82 | 22 | 22,292,190 | 22,292,242 | GGACTCCCCATGCCAAGGGC | 142 |
| | | | | TGCAGCCCCGCAACCTCGCTT | |
| | | | | CTGGATTCTTCG | |
| set1.83 | 22 | 29,427,824 | 29,427,887 | GGCAACAGAAACAGGGCTGG | 143 |
| | | | | TTCCTGCCGCCCTGCATTTCA | |
| | | | | GCAGTGACGTGTTCCAGGCTC | |
| | | | | CG | |
| set1.84 | 22 | 37,736,873 | 37,736,939 | CGGTGGTGCCAACTCCATGAT | 144 |
| | | | | ACAGATGAGAAAAGTGAGGC | |
| | | | | CCAGGCGAGGCAATGGGCAC | |
| | | | | GTGGAC | |
| set1.85 | 22 | 39,651,424 | 39,651,479 | CGGAGACGCGTCCCTGCCCTC | 145 |
| | | | | TCAGAGTTGACAGTCCAGAG | |
| | | | | GCAAAAAGGACAATC | |

TABLE 5
Eleven candidate aging targets

| Candidate | | | | | SEQ ID |
|---|---|---|---|---|---|
| Locus | Chromosome | Start | End | Sequence | NO. |
| set2.01 | 2 | 236,044,737 | 236,044,880 | CGATGTGCGGGATGGAGGCC | 146 |
| | | | | CAGAGCTGTTCATCCCTGCAA | |
| | | | | CCAATGTTCACGCAACCACC | |
| | | | | AGGGGGCGAAAGGACTCTAA | |
| | | | | CCCCACACGTAGTGAGTGGT | |
| | | | | TCCCACGCCACGTTCCAGTAG | |
| | | | | GAGAAATGAAGTTCCCGGGG | |
| | | | | AC | |
| set2.02 | 3 | 51,741,261 | 51,741,413 | GGGCCCTCAAGTTGTGGGGC | 147 |
| | | | | GCCCGCGCTGCTGTGTCCAG | |
| | | | | ACAGCGTTCCCTGAGAGCTC | |
| | | | | CGGGAAGCGGGAAGACAGCC | |
| | | | | CCGGGCGTCCCGCCTTTCTTC | |
| | | | | TCCAGAAAACGCACGCCCCA | |
| | | | | CATCGCACTCCCCCGTTCCTC | |
| | | | | CTGCTCCAGCG | |
| set2.03 | 3 | 157,812,185 | 157,812,355 | GGATGCACTGGTCCCACAGG | 148 |
| | | | | CCGTGCCCGAGTGGAGCACT | |
| | | | | GCGAATGGGGCCAAGAAATT | |
| | | | | TTGGCCTTTCTCGCCGGACCT | |
| | | | | GGCTGCCTCCGCGGGCCTCTC | |
| | | | | CGCCTACCGCGCTCCCGCCGC | |
| | | | | GGCCCGACTCCCGCGGGTCT | |
| | | | | CCGCGCCGAACCCACCTGGC | |
| | | | | TCCTATCG | |
| set2.04 | 4 | 41,747,851 | 41,747,944 | GGCTTTGGCACCGTTGGGTCT | 149 |
| | | | | TTGGAGCGAAGATAGGACGC | |
| | | | | TGGCGAAGGGACCCCCAAGC | |
| | | | | GAATCCGGGATGGAGGTGAT | |
| | | | | GGGGCCGGGGCCG | |
| set2.05 | 4 | 147,558,432 | 147,558,491 | GCGCGCCAGCGCCTCTCAGG | 150 |
| | | | | CCTGCCGCCTGCTCTCGCACC | |
| | | | | TGCTCGCCTTCCCCAGGCG | |
| set2.06 | 6 | 37,616,783 | 37,617,010 | CGAGCCCACATCGTTGGAGA | 151 |
| | | | | CGCTGCACTCGTAGCTGCCGC | |
| | | | | TGCTGTCGCGAGTTACGGCGT | |
| | | | | CGAGGCGCAGCTCCGCGTGA | |
| | | | | TCCGGCGCCTCGGCGGCGGC | |
| | | | | GGGAACAACAGGCGGCGGCG | |
| | | | | GCAGCAGCTGCCCTTTGAAA | |
| | | | | CGCCACACAGCCGAGGCGAT | |
| | | | | GCGCTGGGGGCTGCCTCGCA | |
| | | | | GCAGCGAGCAGCGCAGGAGC | |
| | | | | ACGGGCCGGCCCAGCGCCTG | |
| | | | | GCGCAC | |
| set2.07 | 7 | 100,231,577 | 100,231,672 | CGCCTCGGCCTCCCAAAGTG | 152 |
| | | | | CTGGCATTACAGGCGCGAGC | |
| | | | | CGCAGTGCAGGGCCCTCCGC | |
| | | | | GGACCCATTTTCTCCCATCAC | |
| | | | | CACCAGGGCGGCGCC | |
| set2.08 | 7 | 130,418,034 | 130,418,234 | CGGCGAGCATGCTTGGTCAG | 153 |
| | | | | GTGGTCGCTCCGGGAGAACT | |
| | | | | GCTTGGGGCAGAGGGGGCAG | |
| | | | | GAGAAGCGCTTCTCGCCCGT | |
| | | | | GTGCGTCCTGTAGTGGCGGG | |
| | | | | CCAGCTCGTCGGAACGCGTA | |
| | | | | AACTTCTTGTCGCAGTCGAGC | |
| | | | | CAGTCGCAGGAGAAAGGGCG | |
| | | | | CTCACCCGTGTGGGTGCGCTG | |
| | | | | GTGGGACTTGAGGTGCGAC | |
| set2.09 | 13 | 53,775,387 | 53,775,468 | CGGACAGGGACTCGGGCGCC | 154 |
| | | | | AACCGGCAGATGCGCTGCGC | |
| | | | | CCTCTACTGGCAGGTGCACTT | |
| | | | | CCGGCTGCAGCGGGCCTACG | |
| | | | | C | |
| set2.10 | 15 | 31,775,435 | 31,775,543 | CGACACAATGGAAAAGAAAT | 155 |
| | | | | CCTCGAAGGTAGAACCTCGC | |
| | | | | CGCCCGCGCCGCGCCGCGCC | |
| | | | | GCTCAGGGCCGGGCCCCGCG | |
| | | | | CGCCTCGCGCCGCCGCCGCA | |
| | | | | GCTCCTCGC | |
| set2.11 | 19 | 52,104,806 | 52,104,964 | GCTCTGCCAGGCCTGCTCCCT | 156 |
| | | | | CCTGGACGGCCTGAACCGCG | |
| | | | | GTCGGCCCCGCCTGGCCATC | |
| | | | | GGCAAGGGCCGCCGGGGGCT | |
| | | | | GGACGAGGAGGCGACGCCGG | |
| | | | | GGACGCCCGGGGATCCGGCC | |
| | | | | CGGGCCCCCACTTCCGAGAC | |
| | | | | CGTCCCCACCTTCTAGCG | |

### Blood Cell Subpopulation Isolation

For blood cell subpopulation analysis (B cells, T cells, NK cells, Monocytes, and Granulocytes), 6 whole blood samples (3 males and 3 females, age range 45-70 years, median 62.5 years) were purchased from BioIVT (Hicksville, NY). Blood cells were separated using centrifugation over a Ficoll-Paque Plus (Cytiva, Marlborough, MA, USA) gradient. Granulocytes were isolated from the pellet after lysis of red blood cells in isotonic ammonium chloride. Magnetic microbeads and MiniMacs columns were used for isolating blood cell subpopulations from the mononuclear fraction. First, B cells were selected by binding to anti-CD19 microbeads (Miltenyi Biotec, 130-050-301). Sequentially, from the negative flow through fractions of T cells by anti-CD3 microbeads (Miltenyi Biotec, 130-050-101), NK cells by anti-CD56 microbeads (130-050-401), and finally monocytes using anti-CD14 microbeads (130-050-201) were isolated.

### White Blood Cell Isolation and DNA Extraction

Blood samples were collected into 5 ml K₂EDTA tubes. Red blood cells were lysed using isotonic ammonium chloride and white blood cells were collected by centrifugation. The WBC pellets were lysed a solution of 2% SDS and 25 mM EDTA for subsequent DNA extraction. DNA was isolated by lysing the cells in a solution containing 2% SDS and 25 mM EDTA pH 8.0, followed by precipitation of proteins with ammonium acetate (2.5 M final). The protein/SDS precipitate was removed by centrifugation. DNA was precipitated from the clear supernatant by isopropanol, washed with 70% ethanol, and dissolved in TE (TRIS 10 mM, EDTA 1 mM, pH 8.0).

### Multiplex Bisulfite PCR

Primer 3 online tool (https://primer3.ut.ee) was used to design primers for bisulfite converted DNA sequence (Table 2). Bisulfite conversion of DNA was performed with EZ-96 DNA Methylation-Lightning Kit (Zymo Research) or EpiTect Bisulfite Kit (Qiagen, Germany). Multiplex PCR was performed with Platinum multiplex PCR master mix (Applied Biosystems, CA, USA) following the manufacturer's recommendations. The final concentration of each primer in the multiplex PCR was adjusted to 200 nM. We used PCR with an initial denaturation (94° C., 2 min) followed by 35 cycles of denaturation (94° C., 30 s), annealing (60° C., 4 min), and extension (72° C., 30 s). The PCR was concluded by a final extension (72° C., 5 min), and holding at 4° C.

### Deep Sequencing of Bisulfite PCR Amplicons

The PCR amplicons were cleaned using SPRI beads at 2× beads to PCR product ratio to remove primers and small unspecific PCR products. Next, we used the NEBNext Ultra II DNA Library Prep Kit from Illumina (NEB E7645, MA, USA) to 5′-phosphorylate and 3′-adenylate the PCR products and ligate the sequencing adapters. The resulting libraries were cleaned with SPRIselect beads (Beckman Coulter B23318) at 1.2× beads to DNA ratio. Libraries were amplified with 96 pairs of sample-specific dual barcoded NEB primers (NEB E6440) using 6 cycles of PCR. The barcoded libraries were cleaned with SPRIselect beads (Beckman Coulter B23318), at a 1× ratio, quantified the DNA by Qubit HS, and pooled equal DNA amounts from all samples amplified. The size distribution of DNA fragments in the pool was checked by electrophoresis using Agilent DNA 1000 kit (5067-1504) and Agilent 2100 BioAnalyzer, the DNA concentration was verified by Qubit fluorometer, and the molarity of the pool was calculated. The pool was sequenced on the Illumina MiSeq instrument using a MiSeq Reagent Micro Kit v2 (MS-103-1002) and paired end sequencing of 2×150 bases.

### Data Processing and Analysis of Bisulfite Amplicons

The quality of the sequencing reads was first checked by using the FastQC v0.11.9 tool. Next, the reads were trimmed by removing the sequencing adapters and sequences shorter than 100 bp using trim galore 0.6.7. The trimmed reads were aligned to the bisulfite-converted hg19 human genome using Bismark 0.23.1 and obtained read methylation data using bismark_methylation_extractor. The bisulfite conversion efficiency was calculated based on the level of nonCpG methylation, average methylation at the PCR targets, and Jensen-Shannon distance of CpG methylation patterns between the sample and the cord blood reference using the package philentropy and custom R scripts.

### DMC Age Calculation and Model Building

Linear modeling was used to develop a statistical model of “methylation age” using JSD values for the 20 loci. The model was developed in the NINDS samples. Bootstrapping was used to select targets that consistently improved performance of the model when included, by randomizing the order in which targets were sequentially dropped, and then training and testing the model after dropping each target. This process was repeated 1000 times keeping only targets that, if dropped, worsened the performance of the model. After this process obtained were 1000 bootstraps each with a particular set of targets, that returned the best Median Absolute Error. Selected targets kept in >=75% of the best 100 bootstraps, as measured by the Median Absolute Error. To calculate DMC methylation age, the value of JSD was multiplied by 100, then the JSD values were averaged at the final group of targets. After obtaining this one average value, a linear model was fit with average JSD as the response variable, and the chronological age of the sample as the explanatory variable. After obtaining the intercept and coefficient from this regression model the equation was reversed by subtracting the intercept from the average JSD value and dividing by the coefficient to obtain the methylation age. Error was calculated by subtracting the chronological age from the predicted methylation age, and MAE was then calculated by taking the median of the absolute value of the error.

### Clinical Data

To analyze the clinical data, the “glm” function from the stats package in R was used to fit logistic regression models where the response variable was a binary medical history variable (ex: Congestive Heart Failure −1 or 0 for no or yes) and the explanatory variable was the predicted methylation age calculated from our model. The odds ratios were then calculated from the log-odds returned by the model by taking the exponent. This analysis was performed on both a set containing patients of all ages, and a set of only patients that were 60 years old or older.

In addition to the univariate model, also run was a model controlling for only chronological age; one controlling for chronological age, sex, race, and BMI; and a model controlling for all the previous and smoking history. For the Charlson Comorbidity Index (CCI) and Trauma Specific Frailty index (TSFI), Pearson correlations were calculated for patients of all ages and in only for patients 60 years old or older.

### Leukemia Data

When comparing methylation and JSD at the 3 different time points of the leukemia data, paired t-tests were calculated between D0 and D7, D0 and D14, and D7 and D14 (where D0 is the starting day of treatment, D7, and D14 refer to the day of treatment with a hypomethylating drug) in the methylation and JSD values at each target. The model coefficients calculated from the Cooper samples was used to calculate methylation age.

### Selection of Loci

Candidate biomarkers of age-related DNA methylation were selected using RRBS data from 19 NINDS biobank individuals aged 22-80 years and 33 umbilical cord blood samples publicly available at GEO GSE109538. Different algorithms were used to select 20 targets for assay development (Table 6). Sixteen of the 20 targets were in CpG islands, and 7/20 were in promoters/first exons. These data are consistent with genome wide studies suggesting that non-promoter CpG islands were particularly sensitive to age-related DNA methylation changes.

TABLE 6
Targets selected for assay development.

| Target | Chromo- | | | | | # of | CpG | Closest | |
|---|---|---|---|---|---|---|---|---|---|
| ID | some | Start | End | Strand | Span | CpGs | Island | gene | Location |
| C2_193 | 2 | 236,044,716 | 236,044,906 | + | 191 | 9 | No | none | intergenic |
| Ks05 | 3 | 47,051,176 | 47,051,366 | − | 191 | 18 | Yes | NBEAL2 | 3′ end |
| B8_175 | 3 | 51,741,247 | 51,741,421 | − | 175 | 15 | Yes | GRM2 | intron1 |
| B3_180 | 3 | 157,812,179 | 157,812,358 | − | 180 | 21 | Yes | none | orphan |
| | | | | | | | | | CGI |
| B6_151 | 4 | 41,747,818 | 41,747,968 | − | 151 | 9 | Yes | PHOX2B | end |
| R8436 | 4 | 147,558,398 | 147,558,513 | − | 116 | 9 | Yes | POU4F2 | promoter |
| R3988 | 5 | 174,673,908 | 174,674,141 | + | 234 | 3 | No | no | intergenic |
| R05 | 6 | 10,416,394 | 10,416,549 | + | 156 | 8 | No | TFAP2A | promoter |
| T5_275 | 6 | 37,616,759 | 37,617,033 | + | 275 | 34 | Yes | MDGA1 | exon9/17 |
| Ks08 | 7 | 15,725,514 | 15,725,691 | − | 178 | 15 | No | MEOX2 | first exon |
| Ks07 | 7 | 100,231,520 | 100,231,731 | + | 212 | 10 | Yes | TFR2 | promoter |
| T1_200 | 7 | 130,418,062 | 130,418,261 | + | 200 | 21 | Yes | KLF14 | exon1 |
| Ks09 | 8 | 102,504,781 | 102,504,950 | − | 170 | 10 | Yes | GRHL2 | first exon |
| Ks10 | 9 | 1,046,175 | 1,046,301 | + | 127 | 11 | Yes | DMRT2 | intergenic |
| Ks11 | 9 | 136,075,447 | 136,075,649 | + | 203 | 18 | Yes | OBP2B | intergenic |
| R23 | 13 | 49,795,399 | 49,795,541 | − | 143 | 12 | Yes | MLNR | intron |
| C13_194 | 13 | 53,775,359 | 53,775,489 | + | 131 | 10 | Yes | lincRNA | start |
| R5434 | 15 | 31,775,320 | 31,775,568 | + | 249 | 23 | Yes | OTUD7A | exon 11 |
| Ks02 | 17 | 40,700,537 | 40,700,703 | − | 167 | 18 | Yes | HSD17B1 | intergenic |
| B2_165 | 19 | 52,104,806 | 52,104,964 | − | 159 | 20 | Yes | lincRNA | end |

DNA Methylation and JSD Vs. Age in NINDS Data

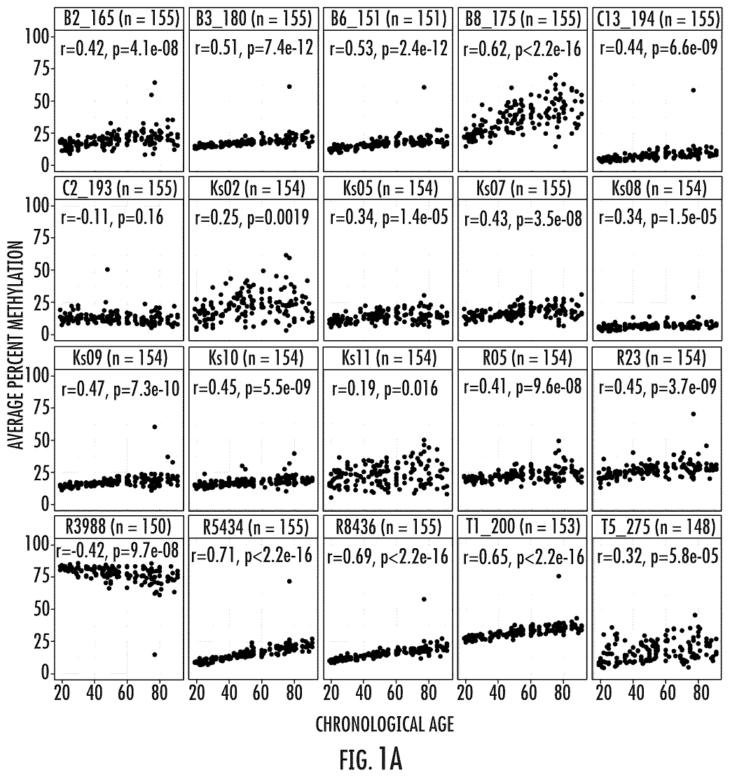

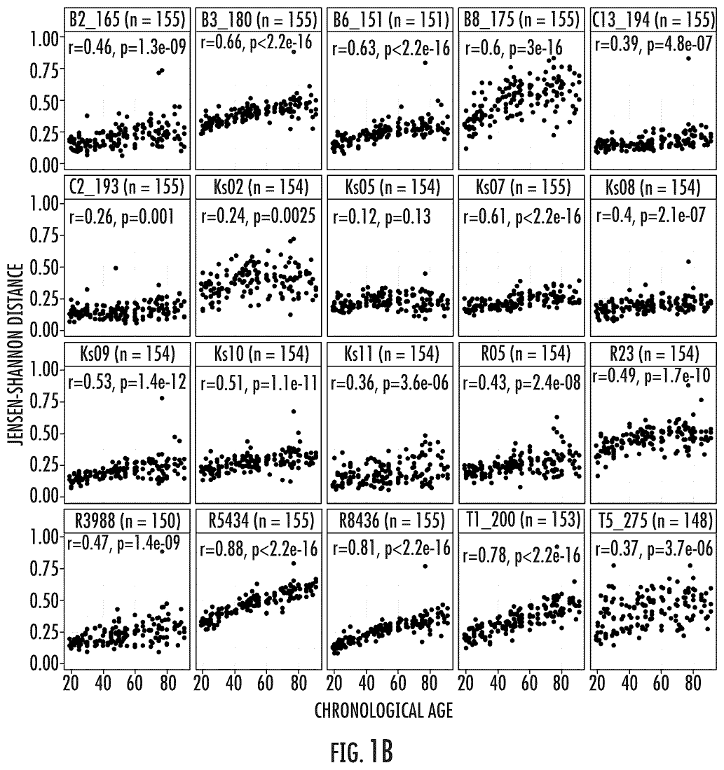

To develop a cost-effective and rapid assay based on the selected target loci, a bisulfite-multiplex PCR assay that combined all primers in a single tube format was used. Primer sequences are shown in Table 2. PCR-amplified DNA was barcoded and sequenced on the Illumina next-generation sequencing platform. Peripheral blood DNA from 155 control individuals obtained from the NINDS biobank (Table 3) was studied. Ages ranged from 19 to over 90 and 77 (50%) were female. The 20 targets were detected in with a median of 737 reads/locus/patient (range 1 to 12,693). Percent DNA methylation was averaged across all CpG sites in each locus and correlated with age of the individuals. FIG. 1A shows DNA methylation vs. age for all targets and Table 7 shows Pearson r values and p-values for these correlations. All but one target showed significant correlations with age, with R values ranging from −0.41 to 0.71.

TABLE 7
Pearson correlations between DNA methylation (left) or JSD (right)
and chronological age for all 20 targets in the discovery dataset.

| | |
|---|---|
| Methylation vs. Age | JSD vs. Age |

| Target | Pearson r | P-value | Pearson r | P-value |
|---|---|---|---|---|
| B2_165 | 0.42 | 4.12E−08 | 0.46 | 1.26E−09 |
| B3_180 | 0.51 | 7.41E−12 | 0.66 | 5.35E−21 |
| B6_151 | 0.53 | 2.42E−12 | 0.63 | 5.31E−18 |
| B8_175 | 0.62 | 5.21E−18 | 0.6 | 2.95E−16 |
| C13_194 | 0.44 | 6.63E−09 | 0.39 | 4.82E−07 |
| C2_193 | −0.11 | 0.156 | 0.26 | 0.00104 |
| Ks02 | 0.25 | 0.00194 | 0.24 | 0.0025 |
| Ks05 | 0.34 | 1.36E−05 | 0.12 | 0.139 |
| Ks07 | 0.43 | 3.48E−08 | 0.61 | 3.21E−17 |
| Ks08 | 0.34 | 1.52E−05 | 0.40 | 2.06E−06 |
| Ks09 | 0.47 | 7.29E−10 | 0.53 | 1.37E−12 |
| Ks10 | 0.45 | 5.49E−09 | 0.51 | 1.09E−11 |
| Ks11 | 0.19 | 0.0159 | 0.36 | 3.59E−06 |
| R23 | 0.45 | 3.72E−09 | 0.47 | 1.10E−09 |
| R3988 | −0.42 | 9.69E−08 | 0.47 | 1.39E−09 |
| R5434 | 0.71 | 1.83E−25 | 0.88 | 2.47E−51 |
| R05 | 0.41 | 9.59E−08 | 0.43 | 2.41E−08 |
| R8436 | 0.69 | 6.77E−23 | 0.81 | 1.50-37 |
| T1_200 | 0.65 | 4.82E−20 | 0.78 | 3.81E−33 |
| T5_275 | 0.32 | 5.76E−05 | 0.37 | 3.70E−06 |

To measure DNA methylation chaos, JSD as previously described was used. JSD values were generated for each locus by comparing the distribution of methylated CpG sites in alleles to that seen in 2 cord blood samples used as a control. FIG. 1B shows JSD vs. age for all loci and Table 7 shows Pearson r values and p-values for these correlations. All but one locus showed significant correlations with age. Interestingly, the r values for JSD were higher than those for percent methylation in most of the loci. Given that JSD is agnostic of percent methylation, these data suggest that chaos is a better measure of age-related epigenetic disruption than average percent methylation, as we have previously shown in mice (Vaidya H, Jeong H S, Keith K, Maegawa S, Calendo G, Madzo J, Jelinek J, Issa J J. DNA methylation entropy as a measure of stem cell replication and aging. Genome Biol. 2023 Feb. 16; 24 (1): 27. doi: 10.1186/s13059-023-02866-4.).

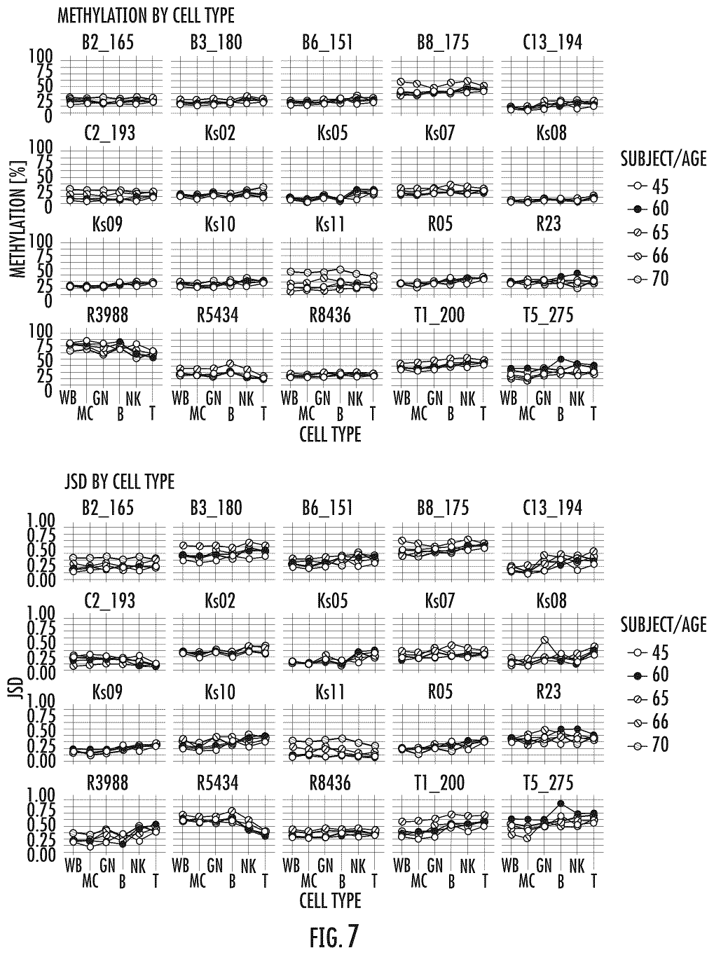

Whole blood consists of a mixture of different cell types which have distinct DNA methylation patterns for selected loci associated with differentiation. Although aging and differentiation loci are largely distinct, it was still sought to determine whether the 20 loci selected show differentiation specific DNA methylation patterns in whole blood. Blood derived from 6 individuals was separated into B-cells, T-cells, NK-cell, monocytes, and granulocytes. The DNA methylation assay was applied to DNA derived from all these samples. FIG. 7 shows the DNA methylation and JSD values for all loci by cell type, with no major cell type specific patterns. There were trends for lower DNA methylation and JSD in T-cells for the R3988 locus, and lower JSD for T-cells in the R5434 locus. As shown in Table 8, paired t-tests between whole blood and each specific cell type were not significant after adjusting for multiple testing for all but one locus. These results suggest that the observed patterns were indeed specific to aging, rather than differentiation.

TABLE 8
White blood cell composition does not affect the results of the DMC aging assay.
No statistically significant differences were observed between the whole blood
and blood cell subpopulations for DNA methylation and JSD at most targets.
P-values from paired t-tests between DNA methylation (A) and JSD (B) of whole
blood and each specific cell type are shown. WB, whole blood; MC, monocytes;
GN, granulocytes, B, B cells; NK, natural killer cells; T, T cells.

| | | | | | |
|---|---|---|---|---|---|
| Target | WB_vs_MC | WB_vs_GN | WB_vs_B | WB_vs_NK | WB_vs_T |

A. Difference in target methylation across blood cell types.

| | | | | | |
|---|---|---|---|---|---|
| Ks02 | 1 | 1 | 1 | 1 | 1 |
| Ks05 | 1 | 0.6 | 1 | 1 | 0.3 |
| Ks07 | 1 | 1 | 1 | 1 | 1 |
| Ks08 | 1 | 1 | 1 | 1 | 1 |
| Ks09 | 1 | 1 | 1 | 1 | 0.4 |
| Ks10 | 1 | 1 | 1 | 1 | 1 |
| Ks11 | 1 | 1 | 1 | 1 | 1 |
| R23 | 1 | 1 | 1 | 1 | 1 |
| R3988 | 1 | 1 | 1 | 1 | 1 |
| R5434 | 1 | 1 | 1 | 1 | 0.3 |
| R05 | 1 | 1 | 1 | 1 | 0.003 |
| R8436 | 1 | 1 | 1 | 0.9 | 1 |
| B2_165 | 1 | 1 | 1 | 1 | 1 |
| B3_180 | 1 | 1 | 1 | 0.7 | 0.8 |
| B6_151 | 1 | 1 | 1 | 1 | 0.2 |
| B8_175 | 1 | 1 | 1 | 1 | 1 |
| C13_194 | 1 | 1 | 1 | 1 | 0.2 |
| C2_193 | 1 | 1 | 1 | 1 | 1 |
| T1_200 | 1 | 1 | 0.9 | 0.3 | 0.1 |
| T5_275 | 1 | 1 | 1 | 1 | 1 |

B. Difference in JSD across blood cell types.

| | | | | | |
|---|---|---|---|---|---|
| Ks02 | 1 | 1 | 1 | 1 | 1 |
| Ks05 | 1 | 1 | 1 | 1 | 0.4 |
| Ks07 | 1 | 1 | 1 | 1 | 1 |
| Ks08 | 1 | 1 | 1 | 1 | 0.5 |
| Ks09 | 1 | 1 | 1 | 1 | 0.5 |
| Ks10 | 1 | 1 | 1 | 1 | 1 |
| Ks11 | 1 | 1 | 1 | 1 | 1 |
| R23 | 1 | 1 | 1 | 1 | 1 |
| R3988 | 1 | 1 | 1 | 1 | 1 |
| R5434 | 1 | 1 | 1 | 0.5 | 0.08 |
| R05 | 1 | 1 | 1 | 1 | 0.009 |
| R8436 | 1 | 1 | 1 | 1 | 1 |
| B2_165 | 1 | 1 | 1 | 1 | 1 |
| B3_180 | 1 | 1 | 1 | 1 | 1 |
| B6_151 | 1 | 1 | 1 | 1 | 0.1 |
| B8_175 | 1 | 1 | 1 | 1 | 1 |
| C13_194 | 1 | 1 | 0.7 | 1 | 0.3 |
| C2_193 | 1 | 1 | 1 | 1 | 1 |
| T1_200 | 1 | 1 | 0.4 | 0.1 | 0.07 |
| T5_275 | 1 | 1 | 1 | 1 | 1 |

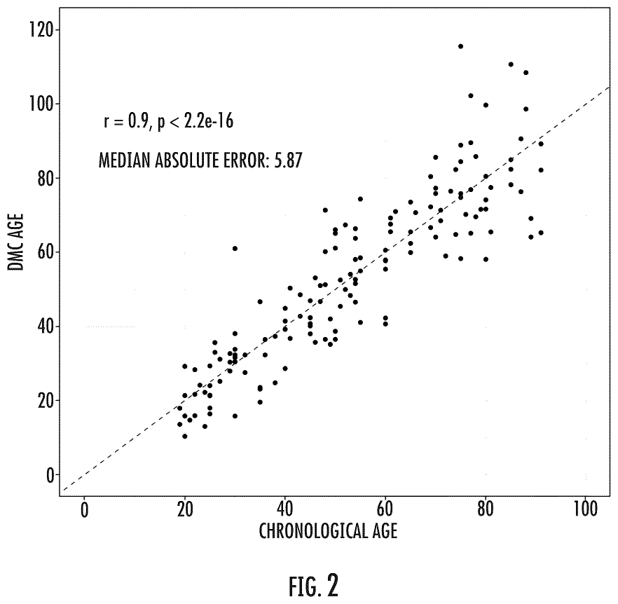

Next used was linear modeling to develop a statistical model of DMC age using JSD values. To improve precision, required for inclusion was a minimum of 40 reads in ≥17/20 loci overall, and ≥5/6 loci among those with the highest Pearson r value. This left 152/155 (98%) evaluable samples in the initial dataset. In samples that were not filtered out, values were inputted for targets with less than forty reads by predicting values using a linear regression model with JSD as the response variable and age as the explanatory variable. For each individual target, a model was trained in samples with greater than forty reads at that target then this model was applied to samples with less than forty reads at the target to give a reasonable imputation of a JSD value based on age. As described, bootstrapping was used to select targets that consistently improved performance of the model when included. The final model included data on seven targets: T1_200, R8436, R5434, C2_193, R3988, Ks07, and Ks11. In building the model, one striking outlier was noticed and a Median Absolute Errors (MAEs) was therefore calculated with or without inclusion of this single case. FIG. 2 shows the correlation between the model's predicted age and chronological age, demonstrating an r value of 0.895 (p<0.001). A similar exercise using average DNA methylation yielded a lowest MAE of 6, consistent with the lower accuracy noted earlier and we did not pursue this model further.

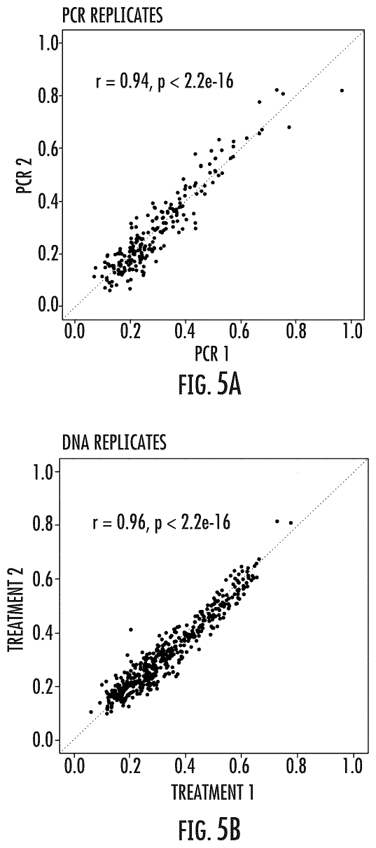

To evaluate reproducibility of the methylation age measurements, 398 pairs of samples were studied where the bisulfite-multiplex-PCR was done in duplicate. As shown in FIG. 5A, the duplicates showed an excellent concordance in methylation age (r=0.96, p<0.001). In addition, 455 pair of samples were studied as full technical replicates (separate bisulfite treatments) and, as shown in FIG. 5B, excellent concordance between DNA methylation ages (r=0.96, p<0.001) was also found. Using limiting dilution, we were able to evaluate the methylation target with DNA input as low as 1.5 nanogram.

DNA Methylation and JSD Vs. Age in a Validation Cohort

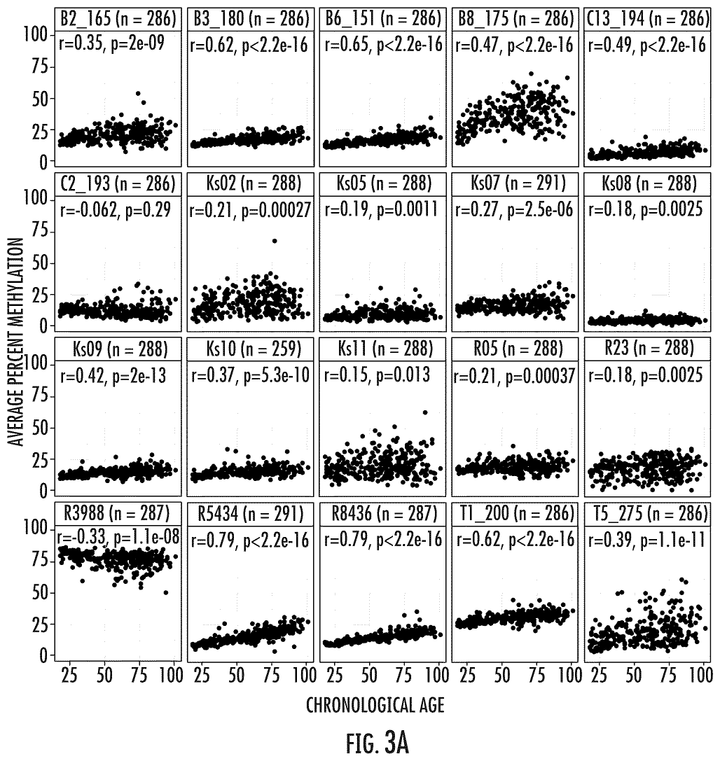

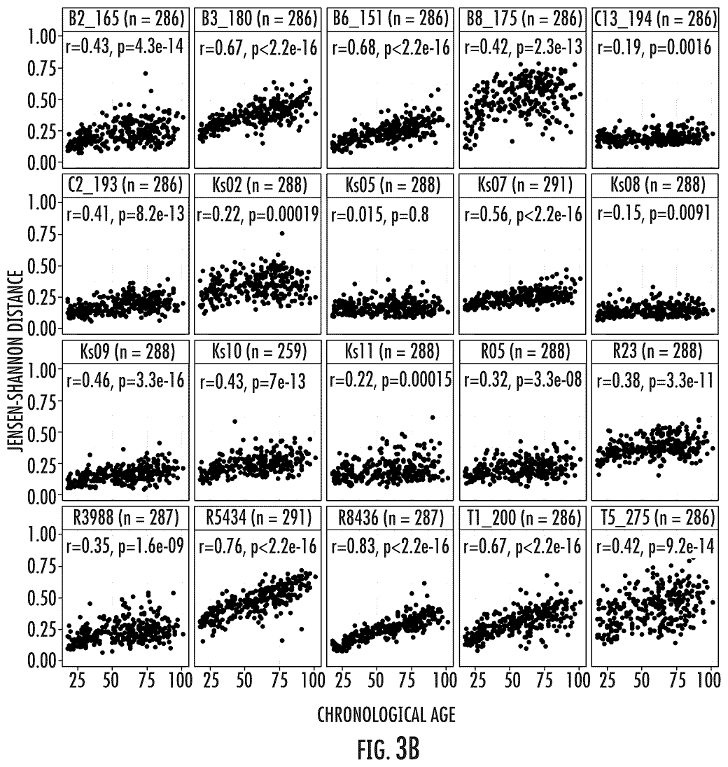

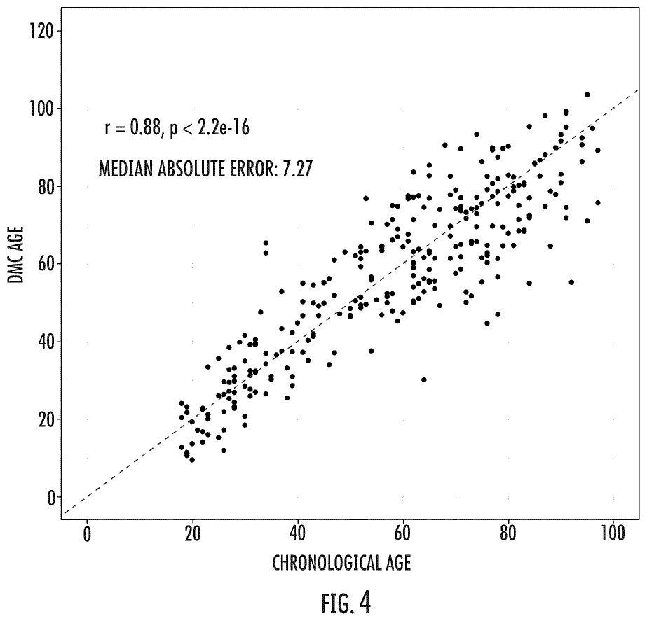

To validate the data obtained using the NINDS samples, 300 patients referred to the Cooper University Hospital (CUH) for management of acute trauma related injuries were studied. Table 3 shows characteristics of the patients studied. In this independent cohort, the 20 loci were detected in all patients with a median of 764 reads/locus/patient (range 1 to 20,690). Percent DNA methylation was again averaged across all CpG sites in each locus and correlated with age of the individuals. FIG. 3A shows DNA methylation vs. age for all loci and Table 9 shows Pearson r values and p-values for these correlations. All but one locus showed significant correlations with age. Next, JSD values were generated for each locus by comparing the distribution of methylated alleles to that seen in two cord blood samples used as a control. FIG. 3B shows JSD vs. age for all loci and Table 9 shows Pearson r values and p-values for these correlations. All but one locus showed significant correlations with age. Interestingly, the r values for both methylation and JSD correlated strongly between the NINDS and the CUH cohorts (r>0.8 for both), thus validating the assay. Once again, JSD r values were generally higher than those for percent methylation. The DMC age was calculated using the model described earlier. First applied was a quality filter wherein samples with fewer than forty reads in more than one of the targets chosen in the final DMC Age model were excluded, which left 283 evaluable individuals of the initial 300 patient cohort (94%). For the entire cohort, MAE was 8.39 (range −37.03, 67.69). FIG. 4 shows a scatter plot of calculated age vs. chronologic age (r=0.866, p<0.001). Thus, these data strongly validate the use of this model for the calculation of DMC age.

TABLE 9
Pearson correlations between DNA methylation (left) or JSD (right)
and chronological age for all 20 targets in the validation dataset.

| | |
|---|---|
| Methylation vs. Age | JSD vs. Age |

| Target | Pearson r | P-value | Pearson r | P-value |
|---|---|---|---|---|
| B2_165 | 0.34 | 3.58E−09 | 0.42 | 7.87E−14 |
| B3_180 | 0.57 | 4.47E−26 | 0.65 | 1.43E−35 |
| B6_151 | 0.6 | 1.40E−29 | 0.66 | 1.45E−36 |
| B8_175 | 0.46 | 2.21E−16 | 0.41 | 6.04E−13 |
| C13_194 | 0.47 | 5.53E−17 | 0.17 | 3.64E−03 |
| C2_193 | −0.06 | 0.292 | 0.39 | 9.25E−12 |
| Ks02 | 0.21 | 2.74E−04 | 0.22 | 1.95E−04 |
| Ks05 | 0.19 | 1.06E−03 | 0.02 | 0.796 |
| Ks07 | 0.26 | 7.26E−06 | 0.55 | 1.10E−24 |
| Ks08 | 0.18 | 2.54E−03 | 0.15 | 9.09E−03 |
| Ks09 | 0.42 | 1.98E−13 | 0.46 | 3.30E−16 |
| Ks10 | 0.37 | 5.26E−10 | 0.43 | 6.97E−13 |
| Ks11 | 0.15 | 0.0127 | 0.22 | 1.46E−04 |
| R23 | 0.18 | 2.53E−03 | 0.38 | 3.28E−11 |
| R3988 | −0.33 | 1.06E−08 | 0.35 | 1.58E−09 |
| R5434 | 0.77 | 6.22E−59 | 0.75 | 5.24E−53 |
| R05 | 0.21 | 3.73E−04 | 0.32 | 3.34E−08 |
| R8436 | 0.77 | 1.70-57 | 0.80 | 9.62E−65 |
| T1_200 | 0.58 | 5.03E−27 | 0.65 | 1.61E−35 |
| T5_275 | 0.39 | 2.39E−11 | 0.42 | 1.60E−13 |

DMC Correlates with Smoking and Chronic Diseases of Aging.

Detailed clinical-pathologic characteristics and medical history information were available for the validation cohort (but not the initial NIDDS cohort). It was therefore examined whether age acceleration (higher DMC age than chronological age) or age deceleration (lower DMC age than chronological age) were associated with clinical features. The was first examined by computing median age error (DMC age minus chronological age) across the variables. Smoking showed strong associations with accelerated aging (AE+1.82, for current smokers, +2.51 for former smokers, −1.72 for non-smokers). There were no associations between AE and sex, race or obesity. Next examined were AE associations with specific diseases, limiting the analyses to diseases that were present in five or more individuals. Overall, the median AE was above 1 for patients affected with 6 diseases examined, while it was below 1 for unaffected patients in all diseases examined (p value=0.03). Individually, the highest AEs were seen for previous stroke. Next analyzed were the data by dividing individuals studied into three cohorts based on AE—decelerated (AE<−12.55, n=39), normal (AE between −12.55 and 12.55), n=204) and accelerated (AE>12.55, n=43). Cochran-Armitage test for trend analysis was used to examine the significance of associations between this aging classification and specific exposures or diseases. A statistically significant trend was found for smoking (p=0.0007). Overall, these data strongly suggest that DMC age can be influenced by lifestyle factors (e.g., smoking) and that it can potentially predict the emergence of chronic diseases of aging.

DNA Methylation and JSD Vs. Age in Leukemia Samples

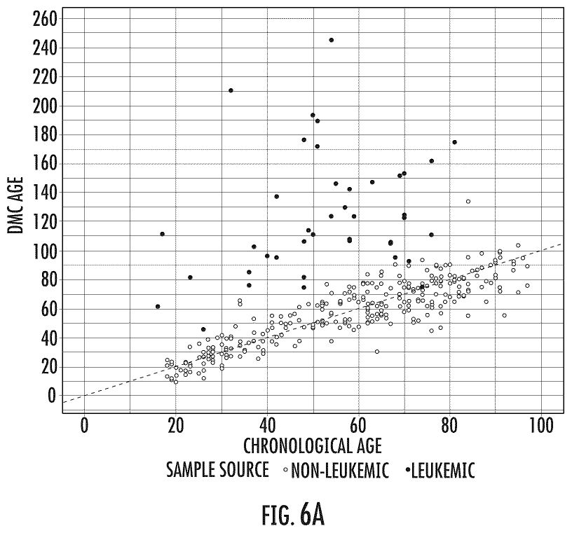

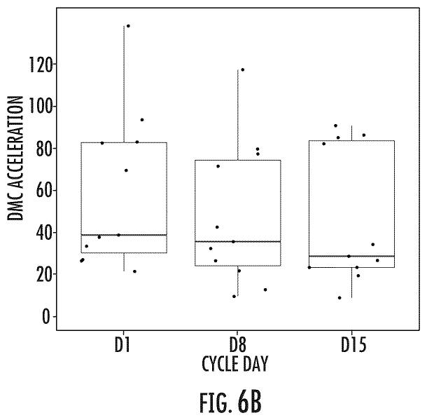

FIG. 1 illustrates at least one case with markedly accelerated aging (DNA methylation age of ˜150). Two factors associated with such acceleration were previously reported-chronic inflammation, and neoplastic transformation. The NINDS samples studied were obtained from a biobank with no clinical information, but DNA methylation of the selected loci was tested in a panel of 40 samples obtained from patients with active Acute and Chronic Myelogenous Leukemia (AML and CML). As shown in FIG. 6A, most of these cases showed markedly accelerated aging (AE range 45.9 270.2 median 121.5) based on the DNA methylation chaos analysis, consistent with previous data. These patients were enrolled in a clinical trial of a DNA hypomethylating drug, allowing testing as to whether the assay could detect in-vivo DNA methylation modulation. As shown in FIG. 6B, AE decreased 7 days and 14 days after treatment with a hypomethylating drug. JSD analysis showed less consistent results when comparing leukemia to normal and especially when comparing post-treatment to pre-treatment. This is very likely due to clonal expansion in leukemias which potentially reduces allelic diversity, highlighting one of the drawbacks of this method of chaos measurement.

Age-related methylation drift is evolutionarily conserved across species, and methylation drift is inversely proportional to longevity. Many groups have used these methylation changes to create epigenetic clocks that estimate biological age, with differences between biological age and estimated age correlating with disease and life expectancy. Some of the clocks developed are used across many different tissues. Some studies have used methylation arrays to study changes in DNA methylation in mice; such arrays could provide a wider range of CpG sites to construct epigenetic clocks or to study tissue specificity. It would be of interest to see to see if the differential methylation analysis results herein can be replicated using such arrays. However, one drawback of using arrays is that one cannot measure chaos using data generated from arrays. The data herein suggest very little overlap in aging changes between distantly related tissues and between tissues that have very different stem cell proliferation rates. While clocks constructed by mixing groups of CpG sites specific for certain tissues may yield assays that work in different tissues, it may be preferable to use tissue-specific clocks for most accurate results. Moreover, the data herein suggest that clocks that measure chaos may provide more accurate measurement of methylation age when compared to clocks based on % methylation.

Although the disclosure has been described with reference to exemplary embodiments, it is not limited thereto. Those skilled in the art will appreciate that numerous changes and modifications may be made to the preferred embodiments of the disclosure and that such changes and modifications may be made without departing from the true spirit of the disclosure. It is therefore intended that the appended claims be construed to cover all such equivalent variations as fall within the true spirit and scope of the disclosure. All referenced journal articles, patents, and other publications cited herein are incorporated by reference herein in their entireties as if fully set forth.

1. A method for determining age of a subject comprising:
(a) calculating a probability distribution of three or more nucleic acid target sequences in cells or biological fluids of the subject;
(b) calculating the level of DNA methylation its probabilistic distribution at each of the nucleic acid target sequences;
(c) determining the age of the subject by comparing the probability distribution of allele chaos within the three or more nucleic acid target sequences relative to a control probability distribution to obtain an average percent methylation and an average Jensen-Shannon distance (JSD) for each nucleic acid target sequence.

2. The method according to claim 1, further comprising:
(i) amplifying DNA from the cells or biological fluids to generate the three or more nucleic acid target sequences to produce amplified DNA and sequencing the amplified DNA to produce sequence data;
(ii) analyzing the sequence data to determine methylation levels at each CpG site;
(iii) calculating an unmethylated CpG average for each of the three or more nucleic acid target sequences;
(iv) calculating epiallele frequencies from (ii) and (iii);
(v) counting the CpGs within the three or more nucleic acid target sequences;
(vi) counting a number of methylated CpGs in the three or more nucleic acid target sequences;
(vii) calculating methylation chaos by determining the average percent methylation and the average Jensen-Shannon distance (JSD) at the three or more nucleic acid target sequences.

3. The method according to claim 2, wherein the amplifying DNA comprises amplifiying at least one of the three or more nucleic acid target sequences with primers comprising one or a pair of primers comprising at least about 75% sequence identity to a sequence in Table 2, optionally one or a pair of primers comprising a sequence in Table 2.

4. The method according to claim 2, wherein at least a portion of the DNA is treated with sodium bisulfite prior to being amplified.

5. The method according to claim 4, wherein the sulfite treated DNA is amplified by the Polymerase Chain Reaction, and optionally wherein the analyzing comprises comparison of the sequence data to non-bisulfite sequence information, further optionally wherein the non-bisulfite sequence information is obtained from one or both of archived genome sequence information or sequencing of amplified, untreated DNA from the cells or biological fluids.

6. The method according to claim 1 wherein the cells are cancer cells.

7. The method according to claim 1, wherein the cells are stem cells.

8. A computer program product encoded on a computer-readable storage medium, wherein the computer program product comprises instructions for:
(a) calculating a probability distribution of three or more nucleic acid target sequences in a cell of the subject;
(b) calculating a percent methylation and a Jensen-Shannon distance (JSD) at each of the nucleic acid target sequences;
(c) determining chaos of DNA methylation by comparing the probability distribution of allele chaos with a control probability distribution to obtain an average percent methylation and an average JSD for each nucleic acid target sequence.

9. The computer program product according to claim 8, further comprising a step of correlating the chaos of DNA methylation with the age of the cell.

10. The computer program product according to claim 9, further comprising instructions for selecting a treatment for the subject based upon the age of the cell.

11. The computer program product according to claim 8, further comprising instructions for:
(d) assigning a score to the amount of chaos of DNA methylation;
(e) comparing the score to a first threshold; and
(f) classifying the subject as being likely to respond to a treatment, if the score exceeds or falls below a first threshold;
wherein each of steps (d), (e), and (f) are performed after step (c), and wherein the first threshold is calculated relative to a first control dataset.

12. A system comprising the computer program product of claim 8 and one or more of:
(a) a processor operable to execute a program; and
(b) a memory associated with the processor.

13. A kit comprising one or more primer complementary to at least one target sequence selected from Tables 1, 4, or 5 and instructions for performing the method of claim 1.

14. The kit of claim 13, wherein the at least one target sequence comprises three target sequences.

15. The kit of claim 13, wherein the at least one target sequence is chosen from Table 1, 4, or 5.

16. The kit of claim 13, wherein the one or more primer comprises at least one set of amplifying primers, each comprising a forward primer and a reverse primer chosen from Table 2 or a variant thereof having at least 75% sequence identity thereto.

17. The kit of claim 13 further comprising one or more reagent for bisulfite sequencing.

18. The kit of claim 13 further comprising a therapeutic agent for delivery to a subject when the subject is determined to have an DMC age greater than actual age.

19. The kit of claim 13 further comprising a computer program product comprising instructions for one or both of (i) sequencing DNA in a cell to obtain at least a portion of nucleic acid sequence of the at least one nucleic acid target sequences; and (ii) analyzing at least a portion of the nucleic acid sequence of the at least one nucleic acid target sequences to determine methylation levels at one or a plurality of CpG sites within the at least one nucleic acid target sequences.

20. A method treating a subject comprising:
(a) calculating a probability distribution of three or more nucleic acid target sequences in cells or biological fluids of the subject;
(b) calculating a level of DNA methylation probabilistic distribution at each of the nucleic acid target sequences;
(c) determining an estimated age of the subject by comparing the probability distribution of allele chaos within the three or more nucleic acid target sequences relative to a control probability distribution to obtain an average percent methylation and an average Jensen-Shannon distance (JSD) for each nucleic acid target sequence; and
(d) administering a hypomethylating drug, anti-inflammatory drug, smoking cessation treatment, administering a GLP1 targeting drug, or a calorie restricted diet to the subject when the estimate age is greater than the actual age of the subject.
